SEQUENCE LISTING (78 sequences)

SEQ ID NO: 1 - DNA, 74 bp, Simian virus 40
ggggactttccggggactttcctccccacgcgggggactttccgccacgggcggggactttccggggactttcc

SEQ ID NO: 2 - DNA, 1497 bp, Artificial Sequence
  synthesized
atggcgaccaaaggcaccaaacgtagctatgaacagatggaaaccgatggcgaacgtcagaacgcgaccgaaattcgtgcgagcgtgggcaaaatgattgatggcattggccgtttttatattcagatgtgcaccgaactgaaactgagcgattatgaaggccgtctgattcagaacagcctgaccattgaacgtatggtgctgagcgcgtttgatgaacgtcgtaacaaatatctggaagaacatccgagcgcgggcaaagatccaaagaaaaccggcggcccgatttatcgtcgtgtggatggcaaatggcgtcgtgaactgattctgtatgataaagaagaaattcgtcgtatttggcgtcaggcgaacaacggcgatgatgcgaccgcgggcctgacccacatgatgatttggcatagcaacctgaacgatgcgacctatcagcgtacccgtgcgctggtgcgtaccggcatggacccacgtatgtgcagcctgatgcagggcagcaccctgccgcgtcgtagcggtgcagcaggtgcagcagtgaaaggcgtgggtacgatggtgatggaactgattcgtatgattaaacgtggcattaacgatcgtaacttttggcgtggcgaaaacggccgtcgtacccgtattgcgtatgaacgtatgtgcaacattctgaaaggcaaatttcagaccgcggcgcagcgtacgatggtggatcaagtgcgtgaaagccgtaacccgggcaacgcggaatttgaagacctgatttttctggcgcgtagcgcgctgattctgcgtggcagcgtggcgcataaaagctgcctgccggcgtgcgtgtatggcagcgcggtggcgagcggctatgattttgaacgtgaaggctatagcctggtgggcattgatccgtttcgtctgctgcagaacagccaggtgtatagcctgattcgtccgaacgaaaacccggcgcataaaagccagctggtgtggatggcgtgccatagcgcggcgtttgaagacctgcgtgtgagcagctttattcgtggcaccaaagtggtgccgcgtggcaaactgagcacccgtggcgtgcagattgcgagcaacgaaaacatggaaacgatggaaagcagcaccctggaactgcgtagccgttattgggcgattcgtacccgtagcggcggcaacaccaaccagcagcgtgcgagcagcggccagattagcattcagccgacctttagcgtgcagcgtaacctgccgtttgatcgtccgaccattatggcggcgtttaccggcaacaccgaaggccgtaccagcgatatgcgtaccgaaattattcgtctgatggaaagcgcgcgtccggaagatgtgagctttcagggccgtggcgtgtttgaactgagcgatgaaaaagcgaccagcccgattgtgccgagctttgatatgagcaacgaaggcagctactttttcggcgataacgcggaagaatatgataactaa

SEQ ID NO: 3 - DNA, 1497 bp, Influenza virus
atggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaa

SEQ ID NO: 4 - protein, 498 aa, Influenza virus
MATKGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWRRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRTMVDQVRESRNPGNAEFEDLIFLARSALILRGSVAHKSCLPACVYGSAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKATSPIVPSFDMSNEGSYFFGDNAEEYDN

SEQ ID NO: 5 - DNA, 28 bp, Artificial Sequence
  synthesized
cggaattcttagcacggtctgcactcat

SEQ ID NO: 6 - DNA, 28 bp, Artificial Sequence
  synthesized
cccgggaattgcttaattgtcgtactcc

SEQ ID NO: 7 - DNA, 26 bp, Artificial Sequence
  synthesized
gtcgaatgctgcgccagttggcgtag

SEQ ID NO: 8 - DNA, 49 bp, Artificial Sequence
  synthesized
cccccatcgatggacggatccccgggaattgcgatgagatcttcgaact

SEQ ID NO: 9 - DNA, 102 bp, Artificial Sequence
  synthesized
gcagtgttgacaaatgaattctccaatttgaatgatgcaacttaccagaggacaagagctcttgttcgcacaggaatggatcccaggatgtgcatcgatgac

SEQ ID NO: 10 - DNA, 24 bp, Artificial Sequence
  synthesized
ccggaattctccaatttgaatgat

SEQ ID NO: 11 - DNA, 32 bp, Artificial Sequence
  synthesized
tccccccgggaattgcttactatttatcgtcg

SEQ ID NO: 12 - DNA, 30 bp, Artificial Sequence
  synthesized
catgccatggcgaccaaaggcaccaaacga

SEQ ID NO: 13 - DNA, 113 bp, Artificial Sequence
  synthesized
tcccccccgggttactatttatcgtcgtcatctttgtagtcgatatcatgatctttataatcaccgtcatggtctttgtagtcattgtcgtactcctctgcattgtctccgaa

SEQ ID NO: 14 - DNA, 21 bp, Artificial Sequence
  synthesized
atgccatggcgatggcgacca

SEQ ID NO: 15 - DNA, 31 bp, Artificial Sequence
  synthesized
ctattaccatgggttatcatattcttccgcg

SEQ ID NO: 16 - DNA, 53 bp, Artificial Sequence
  synthesized
catgccatggctagtggtggtggtggtggtggttatcatattcttccgcgtta

SEQ ID NO: 17 - DNA, 18 bp, Artificial Sequence
  synthesized
attctgaaatgagctgtt

SEQ ID NO: 18 - DNA, 20 bp, Artificial Sequence
  synthesized
tctcatccgccaaaacagcc

SEQ ID NO: 19 - DNA, 7320 bp, Artificial Sequence
  synthesized
agatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaaatggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggagcatgcttatgcgaaaggccatcctgacggatggcctttttggatcttccggaagaccttccattctgaaatgagctgttgacaattaatcatccggctcgtataatgtgtggaattgtgagcggataacaatttcacacaggaaacagaccatggcgaccaaaggcaccaaacgtagctatgaacagatggaaaccgatggcgaacgtcagaacgcgaccgaaattcgtgcgagcgtgggcaaaatgattgatggcattggccgtttttatattcagatgtgcaccgaactgaaactgagcgattatgaaggccgtctgattcagaacagcctgaccattgaacgtatggtgctgagcgcgtttgatgaacgtcgtaacaaatatctggaagaacatccgagcgcgggcaaagatccaaagaaaaccggcggcccgatttatcgtcgtgtggatggcaaatggcgtcgtgaactgattctgtatgataaagaagaaattcgtcgtatttggcgtcaggcgaacaacggcgatgatgcgaccgcgggcctgacccacatgatgatttggcatagcaacctgaacgatgcgacctatcagcgtacccgtgcgctggtgcgtaccggcatggacccacgtatgtgcagcctgatgcagggcagcaccctgccgcgtcgtagcggtgcagcaggtgcagcagtgaaaggcgtgggtacgatggtgatggaactgattcgtatgattaaacgtggcattaacgatcgtaacttttggcgtggcgaaaacggccgtcgtacccgtattgcgtatgaacgtatgtgcaacattctgaaaggcaaatttcagaccgcggcgcagcgtacgatggtggatcaagtgcgtgaaagccgtaacccgggcaacgcggaatttgaagacctgatttttctggcgcgtagcgcgctgattctgcgtggcagcgtggcgcataaaagctgcctgccggcgtgcgtgtatggcccggcggtggcgagcggctatgattttgaacgtgaaggctatagcctggtgggcattgatccgtttcgtctgctgcagaacagccaggtgtatagcctgattcgtccgaacgaaaacccggcgcataaaagccagctggtgtggatggcgtgccatagcgcggcgtttgaagacctgcgtgtgagcagctttattcgtggcaccaaagtggtgccgcgtggcaaactgagcacccgtggcgtgcagattgcgagcaacgaaaacatggaaacgatggaaagcagcaccctggaactgcgtagccgttattgggcgattcgtacccgtagcggcggcaacaccaaccagcagcgtgcgagcagcggccagattagcattcagccgacctttagcgtgcagcgtaacctgccgtttgatcgtccgaccattatggcggcgtttaccggcaacaccgaaggccgtaccagcgatatgcgtaccgaaattattcgtctgatggaaagcgcgcgtccggaagatgtgagctttcagggccgtggcgtgtttgaactgagcgatgaaaaagcgaccagcccgattgtgccgagctttgatatgagcaacgaaggcagctactttttcggcgataacgcggaagaatatgataactaagacccatgggaattcgcaattcccggggatccgtcgacctgcagccaagctcccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtctaga

SEQ ID NO: 20 - DNA, 7320 bp, Artificial Sequence; identical to SEQ ID NO: 19
  synthesized

SEQ ID NO: 21 - DNA, 7392 bp, Artificial Sequence
  synthesized
agatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaaatggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggagcatgcttatgcgaaaggccatcctgacggatggcctttttggatcttccggaagaccttccattctgaaatgagctgttgacaattaatcatccggctcgtataatgtgtggaattgtgagcggataacaatttcacacaggaaacagaccatggcgaccaaaggcaccaaacgtagctatgaacagatggaaaccgatggcgaacgtcagaacgcgaccgaaattcgtgcgagcgtgggcaaaatgattgatggcattggccgtttttatattcagatgtgcaccgaactgaaactgagcgattatgaaggccgtctgattcagaacagcctgaccattgaacgtatggtgctgagcgcgtttgatgaacgtcgtaacaaatatctggaagaacatccgagcgcgggcaaagatccaaagaaaaccggcggcccgatttatcgtcgtgtggatggcaaatggcgtcgtgaactgattctgtatgataaagaagaaattcgtcgtatttggcgtcaggcgaacaacggcgatgatgcgaccgcgggcctgacccacatgatgatttggcatagcaacctgaacgatgcgacctatcagcgtacccgtgcgctggtgcgtaccggcatggacccacgtatgtgcagcctgatgcagggcagcaccctgccgcgtcgtagcggtgcagcaggtgcagcagtgaaaggcgtgggtacgatggtgatggaactgattcgtatgattaaacgtggcattaacgatcgtaacttttggcgtggcgaaaacggccgtcgtacccgtattgcgtatgaacgtatgtgcaacattctgaaaggcaaatttcagaccgcggcgcagcgtacgatggtggatcaagtgcgtgaaagccgtaacccgggcaacgcggaatttgaagacctgatttttctggcgcgtagcgcgctgattctgcgtggcagcgtggcgcataaaagctgcctgccggcgtgcgtgtatggcccggcggtggcgagcggctatgattttgaacgtgaaggctatagcctggtgggcattgatccgtttcgtctgctgcagaacagccaggtgtatagcctgattcgtccgaacgaaaacccggcgcataaaagccagctggtgtggatggcgtgccatagcgcggcgtttgaagacctgcgtgtgagcagctttattcgtggcaccaaagtggtgccgcgtggcaaactgagcacccgtggcgtgcagattgcgagcaacgaaaacatggaaacgatggaaagcagcaccctggaactgcgtagccgttattgggcgattcgtacccgtagcggcggcaacaccaaccagcagcgtgcgagcagcggccagattagcattcagccgacctttagcgtgcagcgtaacctgccgtttgatcgtccgaccattatggcggcgtttaccggcaacaccgaaggccgtaccagcgatatgcgtaccgaaattattcgtctgatggaaagcgcgcgtccggaagatgtgagctttcagggccgtggcgtgtttgaactgagcgatgaaaaagcgaccagcccgattgtgccgagctttgatatgagcaacgaaggcagctactttttcggcgataacgcggaagaatatgataacacatatgtttctgttggtacctctacactggcggcgtatcgtacactggatttccatgattctaacgttaaataagacccatgggaattcgcaattcccggggatccgtcgacctgcagccaagctcccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtctaga

SEQ ID NO: 22 - DNA, 24 bp, Artificial Sequence
  synthesized
gccgggtctagagcctgcagtctg

SEQ ID NO: 23 - DNA, 34 bp, Artificial Sequence
  synthesized
ctagatgttgtgaattctcggagctaggcgccct

SEQ ID NO: 24 - DNA, 41 bp, Artificial Sequence
  synthesized
ccgcgggggcattttctcgtccgggcctgccggtggagtac

SEQ ID NO: 25 - DNA, 41 bp, Artificial Sequence
  synthesized
gtactccaccggcaggcccggacgagaaaatgcccccgcgg

SEQ ID NO: 26 - DNA, 43 bp, Artificial Sequence
  synthesized
caattccaaagcggtggtgccaactcgccggccctgtacctgc

SEQ ID NO: 27 - DNA, 43 bp, Artificial Sequence
  synthesized
gcaggtacagggccggcgagttggcaccaccgctttggaattg

SEQ ID NO: 28 - DNA, 43 bp, Artificial Sequence
  synthesized
gtctagcttctactccgactggtaccagccggcctgcggcaag

SEQ ID NO: 29 - DNA, 43 bp, Artificial Sequence
  synthesized
cttgccgcaggccggctggtaccagtcggagtagaagctagac

SEQ ID NO: 30 - DNA, 43 bp, Artificial Sequence
  synthesized
ctgcaggccaaccgtcacgtcaagccgaccggtagcgccgtcg

SEQ ID NO: 31 - DNA, 43 bp, Artificial Sequence
  synthesized
cgacggcgctaccggtcggcttgacgtgacggttggcctgcag

SEQ ID NO: 32 - DNA, 38 bp, Artificial Sequence
  synthesized
cgctggcgatctatcacccgcagcagttcgtctacgcg

SEQ ID NO: 33 - DNA, 38 bp, Artificial Sequence
  synthesized
cgcgtagacgaactgctgcgggtgatagatcgccagcg

SEQ ID NO: 34 - DNA, 43 bp, Artificial Sequence
  synthesized
ctacgcgggtgcgatgtcgggcctgctggacccgtcccaggcg

SEQ ID NO: 35 - DNA, 43 bp, Artificial Sequence
  synthesized
cgcctgggacgggtccagcaggcccgacatcgcacccgcgtag

SEQ ID NO: 36 - DNA, 43 bp, Artificial Sequence
  synthesized
gctggacccgtcccaggcgatgggtccgaccctgatcggcctg

SEQ ID NO: 37 - DNA, 43 bp, Artificial Sequence
  synthesized
caggccgatcagggtcggacccatcgcctgggacgggtccagc

SEQ ID NO: 38 - DNA, 42 bp, Artificial Sequence
  synthesized
cgcaacgacccgctgctgaacgtcggtaagctgatcgccaac

SEQ ID NO: 39 - DNA, 42 bp, Artificial Sequence
  synthesized
gttggcgatcagcttaccgacgttcagcagcgggtcgttgcg

SEQ ID NO: 40 - DNA, 43 bp, Artificial Sequence
  SYNTHESIZED
caagttcctcgagggcttcgtgcgtaccagcaacatcaagttc

SEQ ID NO: 41 - DNA, 43 bp, Artificial Sequence
  SYNTHESIZED
gaacttgatgttgctggtacgcacgaagccctcgaggaacttg

SEQ ID NO: 42 - DNA, 42 bp, Artificial Sequence
  SYNTHESIZED
caacgctatgaagccggacctgcaacgtgcactgggtgccac

SEQ ID NO: 43 - DNA, 42 bp, Artificial Sequence
  SYNTHESIZED
gtggcacccagtgcacgttgcaggtccggcttcatagcgttg

SEQ ID NO: 44 - DNA, 43 bp, Artificial Sequence
  SYNTHESIZED
gggtgccacgccgaacaccggtccggcgccgcagggcgcctag

SEQ ID NO: 45 - DNA, 43 bp, Artificial Sequence
  SYNTHESIZED
ctaggcgccctgcggcgccggaccggtgttcggcgtggcaccc

SEQ ID NO: 46 - DNA, 37 bp, Artificial Sequence
  SYNTHESIZED
gcacggcgaccgcggaattctttctcgtccgggcctg

SEQ ID NO: 47 - DNA, 41 bp, Artificial Sequence
  SYNTHESIZED
ggttccaaagatttctcggtcgacggcgccctgcggcgccg

SEQ ID NO: 48 - DNA, 40 bp, Artificial Sequence
  SYNTHESIZED
aaggatcaccatggggacaaaaataactttatctccccag

SEQ ID NO: 49 - DNA, 42 bp, Artificial Sequence
  SYNTHESIZED
ttctctcacccgggaaaacagcgcggccgcgcctatcagaag

SEQ ID NO: 50 - DNA, 37 bp, Artificial Sequence
  SYNTHESIZED
gaattgtgagcggccgccaatttcacacaggaaacag

SEQ ID NO: 51 - DNA, 36 bp, Artificial Sequence
  SYNTHESIZED
ctgaaaatcttctctcacccgggaaaacagccaagc

SEQ ID NO: 52 - DNA, 97 bp, Artificial Sequence
  SYNTHESIZED
catggggaaagttaaagtactgtccctcctggtaccagctctgctggtggcgggcgcagcgaatgcggctgaattcctgcagccaagcttcccgggt

SEQ ID NO: 53 - DNA, 97 bp, Artificial Sequence
  SYNTHESIZED
agctacccgggaagcttggctgcaggaattcagccgcattcgctgcgcccgccaccagcagagctggtaccaggagggacagtactttaactttccc

SEQ ID NO: 54 - DNA, 31 bp, Artificial Sequence
  SYNTHESIZED
gaccgcgagatctttttctcgtccgggcttg

SEQ ID NO: 55 - DNA, 33 bp, Artificial Sequence
  SYNTHESIZED
gatcgatggatatcacctatggtggatttcggc

SEQ ID NO: 56 - DNA, 25 bp, Artificial Sequence
  SYNTHESIZED
ccaagcttcgatatcctagccggcc

SEQ ID NO: 57 - DNA, 42 bp, Artificial Sequence
  SYNTHESIZED
gcataccatggtaaaaggatggtgactaacataacactatcc

SEQ ID NO: 58 - DNA, 25 bp, Artificial Sequence
  SYNTHESIZED
ccaagcttcctgcagctagccggcc

SEQ ID NO: 59 - DNA, 36 bp, Artificial Sequence
  SYNTHESIZED
ccacgagaaatagggccatggaatggtggatttcgg

SEQ ID NO: 60 - protein, 50 aa, Influenza virus
NSKEQQNLYQMENAYVSVVTSNYNRRFTPEIAERPKVRDQAGRMNYYWTL

SEQ ID NO: 61 - protein, 50 aa, Influenza virus
SSDEQQSLYSMGNAYVSVASSNYNRRFTPEIAARPKVKDQHGRMNYYWTL

SEQ ID NO: 62 - protein, 50 aa, Influenza virus
NIGGQKALYHTENAYVSVVSSHYSRKFTPEIAKRPKVRDQEGRINYYWTL

SEQ ID NO: 63 - protein, 50 aa, Influenza virus
DAAEQTKLYQMPTTYVSVGTSTLNQRLVPRIATRSKVNGQSGRMEFFWTI

SEQ ID NO: 64 - protein, 50 aa, Influenza virus; identical to SEQ ID NO: 63

SEQ ID NO: 65 - protein, 50 aa, Influenza virus
DAAEQTKLYQMPTTYISVGTSTLNQRLVPEIATRPKVNGQSGRMEFFWTI

SEQ ID NO: 66 - protein, 50 aa, Influenza virus
ENERTLDFHDSNVKNLYEKVKSQLKNNAKEIGNGCFEFYHKCDNECMESV

SEQ ID NO: 67 - protein, 50 aa, Influenza virus
ENERTLDFHDLNVKNLYEKVKSQLKNNAKEIGNGCFEFYHKCDNECMESV

SEQ ID NO: 68 - protein, 50 aa, Influenza virus
ENERTLDFHDSNVKNLYEKVKSQLKNNAKEIGNGCFEFYHKCNDECMESV

SEQ ID NO: 69 - protein, 50 aa, Influenza virus
ENERTLDFHDSNVKNLYDKVRLQLRDNAKELGNGCFEFYHKCDNECMESV

SEQ ID NO: 70 - protein, 37 aa, Influenza virus
ENERTLDFHDSNVKNLYDKVRLQLRDNAKELGNGCFE

SEQ ID NO: 71 - protein, 50 aa, Influenza virus; identical to SEQ ID NO: 69

SEQ ID NO: 72 - protein, 10 aa, Influenza virus
TYVSVGTSTL

SEQ ID NO: 73 - DNA, 30 bp, Influenza virus
acatatgtttctgttggtacatctacactg

SEQ ID NO: 74 - protein, 11 aa, Artificial Sequence
  SYNTHESIZE
RTLDFHDSNVK

SEQ ID NO: 75 - DNA, 33 bp, Influenza virus
cgtacactggatttccatgattctaacgttaaa

SEQ ID NO: 76 - protein, 3 aa, Artificial Sequence
  SYNTHESIZE
AAY

SEQ ID NO: 77 - DNA, 9 bp, Artificial Sequence
  SYNTHESIZE
gcggcgtat

SEQ ID NO: 78 - DNA, 75 bp, Artificial Sequence
  SYNTHESIZED
acatatgtttctgttggtacctctacactggcggcgtatcgtacactggatttccatgattctaacgttaaataa

(19) United States
(12) Patent Application Publication
(10) Pub. No.: US 2015/0056232 A1
(43) Pub. Date: Feb. 26, 2015

(54) RECOMBINANT BACTERIUM FOR INDUCTION OF CELLULAR IMMUNE RESPONSE

(71) Applicant: Roy Curtiss, III, Paradise Valley, AZ (US)

(72) Inventor: Roy Curtiss, III, Paradise Valley, AZ (US)

(73) Assignee: The Arizona Board of Regents for and on behalf of Arizona State University, Tempe, AZ (US)

(21) Appl. No.: 14/390,306

(22) PCT Filed: Mar. 14, 2013
(86) PCT No.: PCT/US13/31592

§ 371 Date: Oct. 2, 2014

Related U.S. Application Data
(60) Provisional application No. 61/619,307, filed on Apr. 2, 2012.

(51) Int. Cl. C12N 15/74; A61K 39/145; A61K 39/04; C12N 15/70

(52) U.S. Cl. CPC C12N 15/74; C12N 15/70; A61K 39/145; A61K 39/04; A61K 2039/523
USPC 424/186.1; 435/252.3; 424/200.1; 424/190.1

(57) ABSTRACT

The present invention provides a recombinant bacterium and methods of using the recombinant bacterium to induce a cellular immune response.

### GOVERNMENTAL RIGHTS

This invention was made with government support under NIH grant numbers R01 AI065779-06, R01 AI56389 and R01 AI93348. The government has certain rights in the invention.

### FIELD OF THE INVENTION

The present invention provides a recombinant bacterium capable of inducing a Th1 T cell response against an antigen of interest in a host.

### BACKGROUND OF THE INVENTION

Influenza remains one of the most significant diseases worldwide, causing acute respiratory illness and accounting for 25% of the infections that exacerbate chronic lung infections. Several epidemics and three major pandemics have been reported. Influenza infections are primarily and effectively controlled by vaccines that elicit neutralizing antibodies against the surface proteins hemagglutinin (HA) and neuraminidase (NA). Influenza vaccines have to be reformulated annually to match the circulating strains due to antigenic drift and do not protect against strains that arise by antigenic shift due to reassortment of gene segments from different species. The most recent example of this is the emergence of pandemic swine (H1N1) flu in 2009 containing sequences from human, avian and both North American and Eurasian swine origins.

Similarly, Mycobacterium tuberculosis has infected one-third of the human population with some 8 million new infections with disease each year and some 2 million deaths. Many individuals infected undergo remission but potentially undergo reactivation disease latter in life. Tuberculosis is also the number one cause of death in individuals infected with HIV. BCG is a live attenuated vaccine that protects infants from milliary tuberculosis but is without effect in preventing pulmonary tuberculosis in adult populations. It is widely believed that an effective vaccine against tuberculosis will require induction of a strong cellular immunity to M. tuberculosis antigens.

Inactivated vaccines do not generally stimulate cellular immunity. Hence, there is a need in the art for a vaccine technology that would elicit cellular immune responses against M. tuberculosis protective protein antigens and to conserved proteins like the Influenza nucleoprotein (NP) to stimulate an efficient T cell response that would result in clearing bacterial and viral infections. Such a technology would have widespread application in the ultimate control of infectious diseases caused by bacterial, viral and parasite pathogens.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

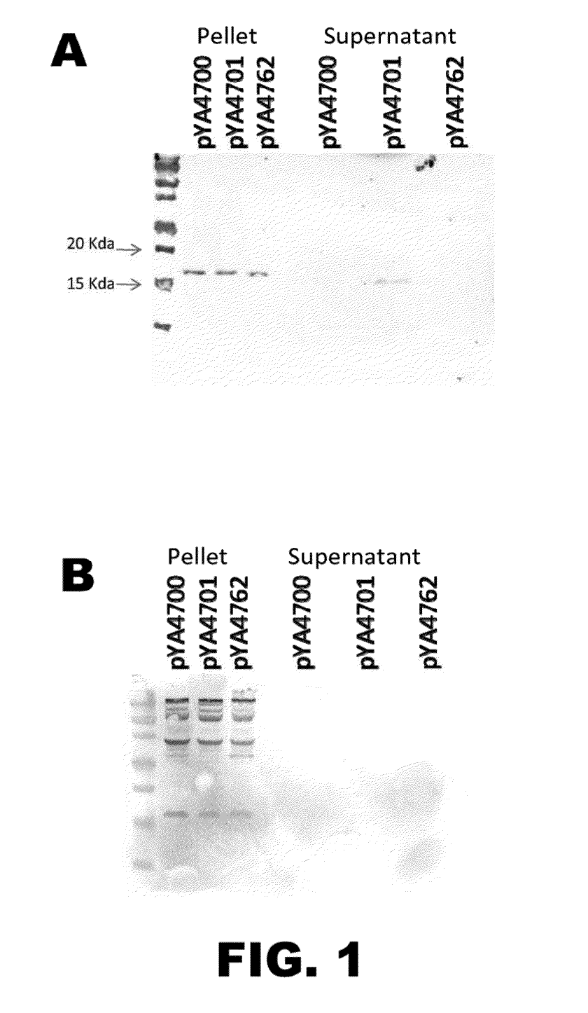

FIG. 1. T3SS secretion analysis. SDS-PAGE/western blot analysis of clones in χ11001 using (A) anti-FLAG antibody and (B) anti-RpoD^{σ70} antibody (cell lysis control).

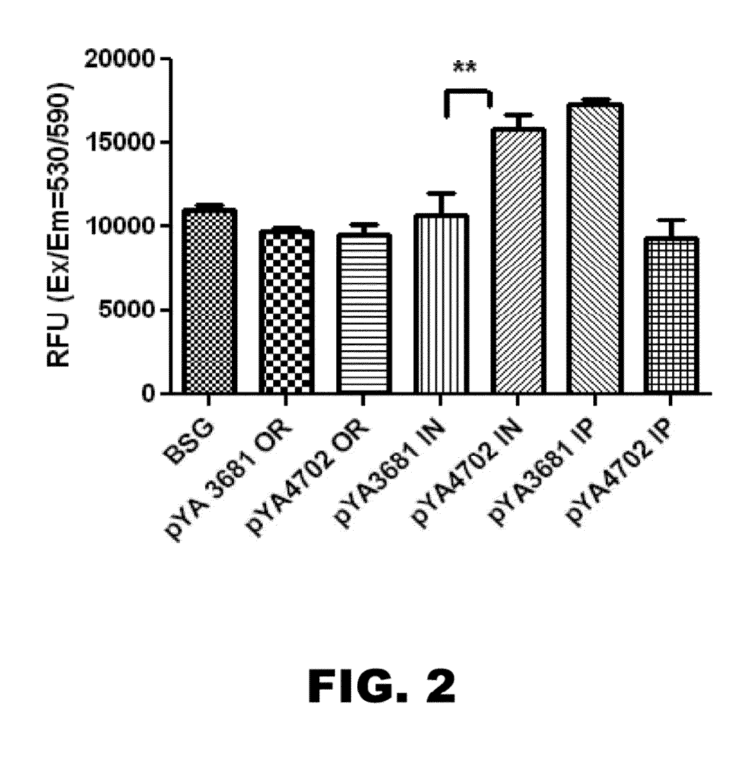

FIG. 2. Analysis of T cell responses elicited by vaccination with NP epitope in lysis vector pYA3681 delivered by the RASV non-lysis strain χ11001 as evaluated by a cell proliferation experiment. BALB/c mice were vaccinated with χ11001(pYA4702) or χ11001(pYA3681) orally, intranasally and intraperitoneally three times. Splenocytes were incubated with 10 μM of Influenza H-2d restricted epitope (NP147-158) for 6 days. Cells were incubated with the Vision blue dye (Biovision) and fluorescence read at 530/590 nm. ** P<0.05. OR=oral; IN=intranasal and IP=intraperitoneal.

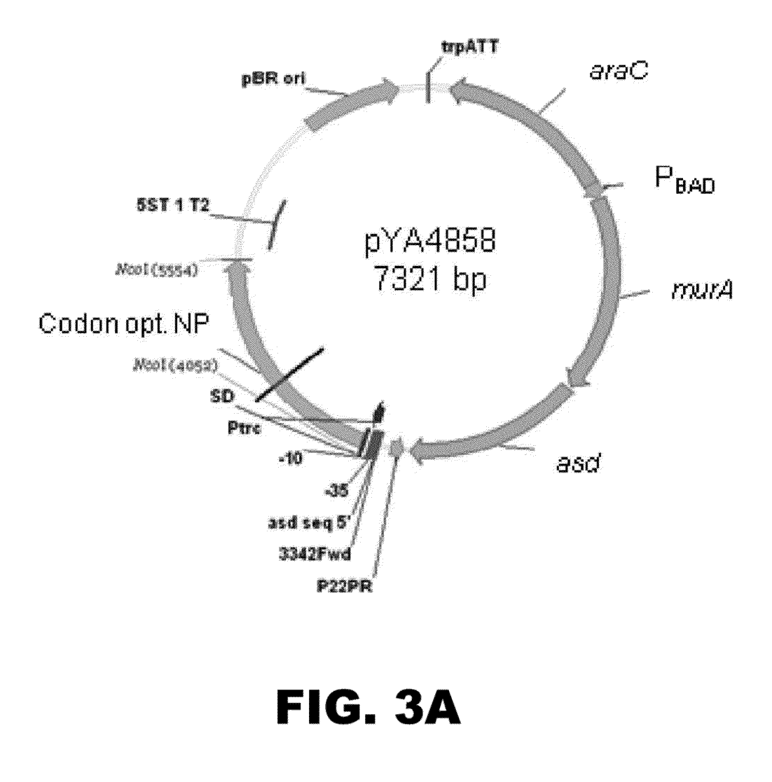

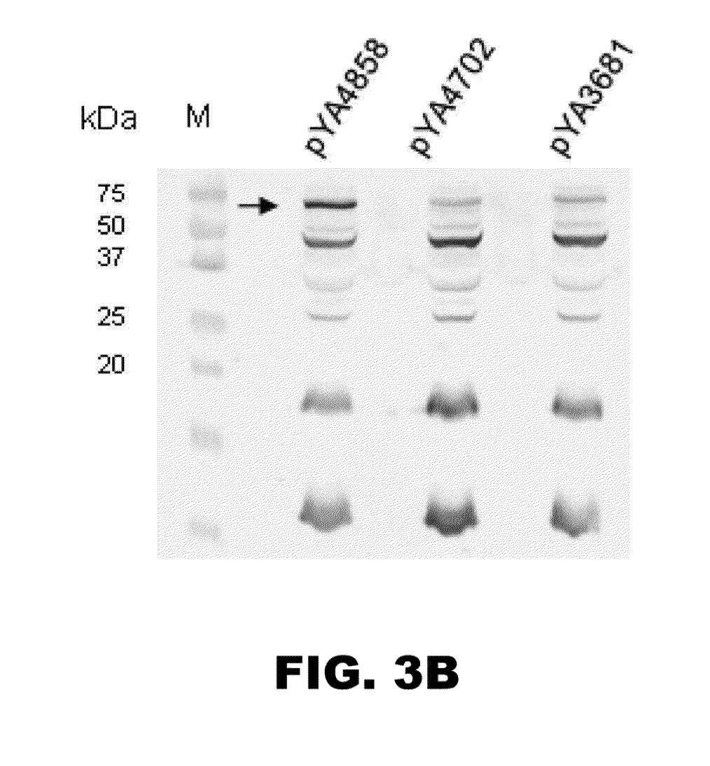

FIG. 3. A. Map of plasmid pYA4858 carrying the codon-optimized NP gene from influenza virus in the regulated delayed lysis plasmid pYA3681. B. Detection of NP in cell free lysates of strain χ11017 (SifA⁺) encoding codon-optimized NP (pYA4858); non-codon-optimized NP (pYA4702) and vector control (pYA3681) using rabbit polyclonal anti-NP sera by western blot analysis. Arrow indicates 60 kDa NP. M=Molecular size marker.

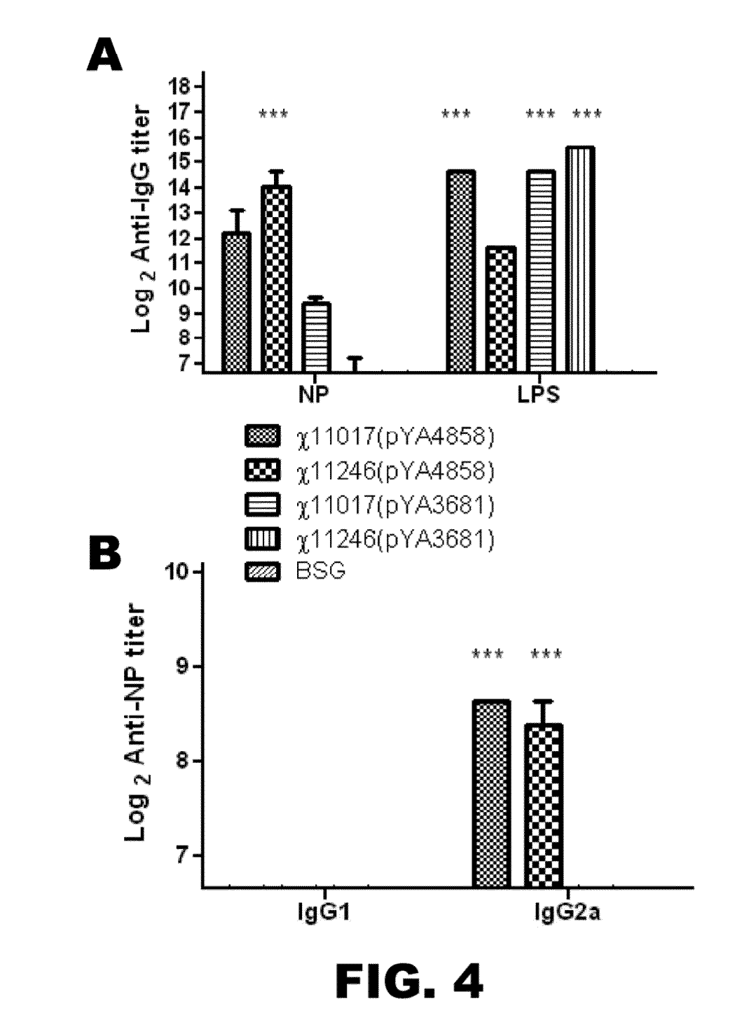

FIG. 4. Trial 1. Antibody titers detected by ELISA in orally immunized mice, 6 weeks after three booster doses with the recombinant attenuated Salmonella strains χ11017(pYA4858) (SifA⁺), χ11246(pYA4858) (SifA⁻) encoding influenza NP or with the vector controls χ11017(pYA3681) (SifA⁺), χ11246(pYA3681) (SifA⁻) or BSG. A. Induction of IgG titers against Influenza NP protein and purified S. Typhimurium LPS. B. Induction of IgG1 and IgG2a responses against influenza NP. Pooled serum samples (n=8) from mice within a group were assayed and analyzed by two-way ANOVA followed by Bonferroni test. ***P<0.001.

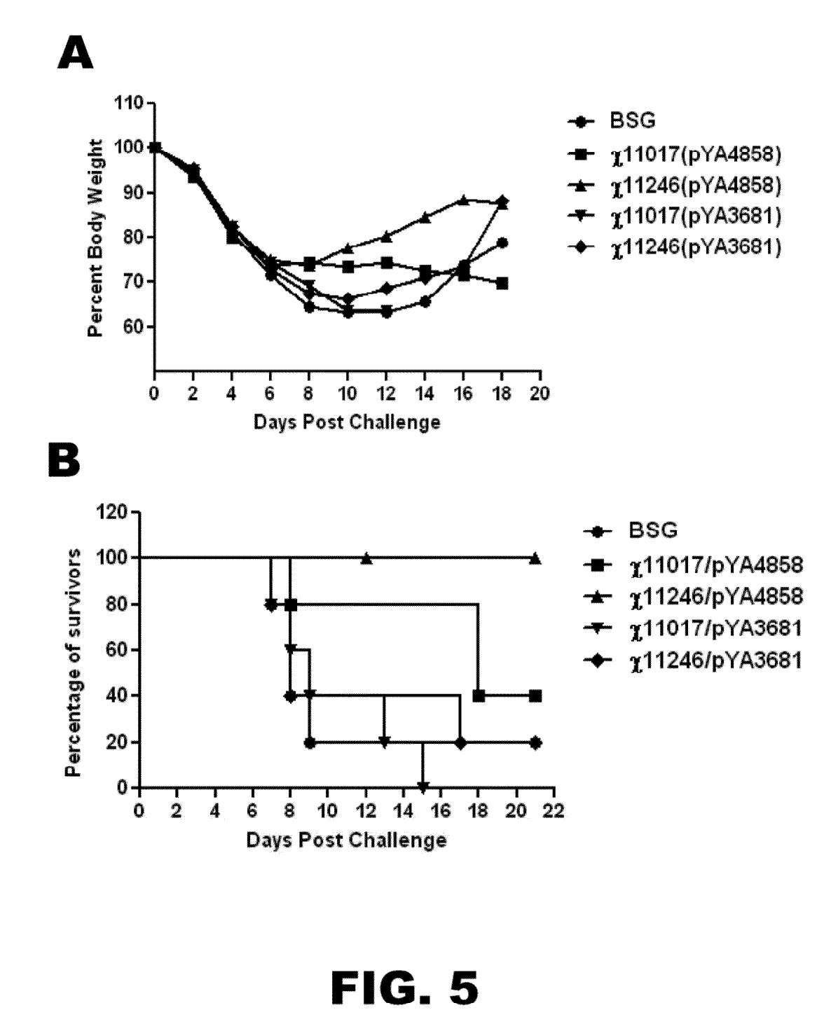

FIG. 5. Trial 1. Weight loss (A) and percent survival (B) of mice (n=5) given three booster oral doses after an intranasal challenge with 100 LD₅₀ of rWSN influenza virus at 8 weeks PPI.

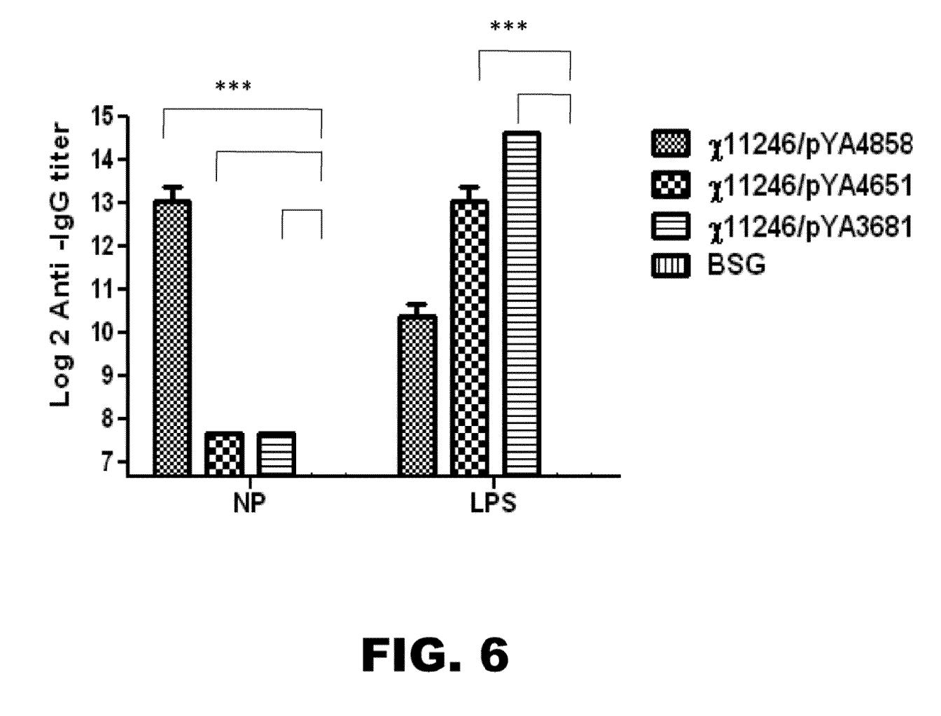

FIG. 6. Trial 2. Induction of IgG titers against influenza NP protein and purified S. Typhimurium LPS as detected by ELISA in orally immunized mice given two booster immunizations, 4 weeks PPI with the recombinant attenuated Salmonella strain χ11246(pYA4858) (SifA⁻) encoding influenza NP or with the negative controls χ11246(pYA4651) (SifA⁻) encoding an irrelevant Ply antigen or the empty vector control χ11246(pYA3681) or BSG. Pooled serum samples (n=3) from mice within a group were assayed and analyzed by two-way ANOVA followed by Bonferroni test. ***P<0.001.

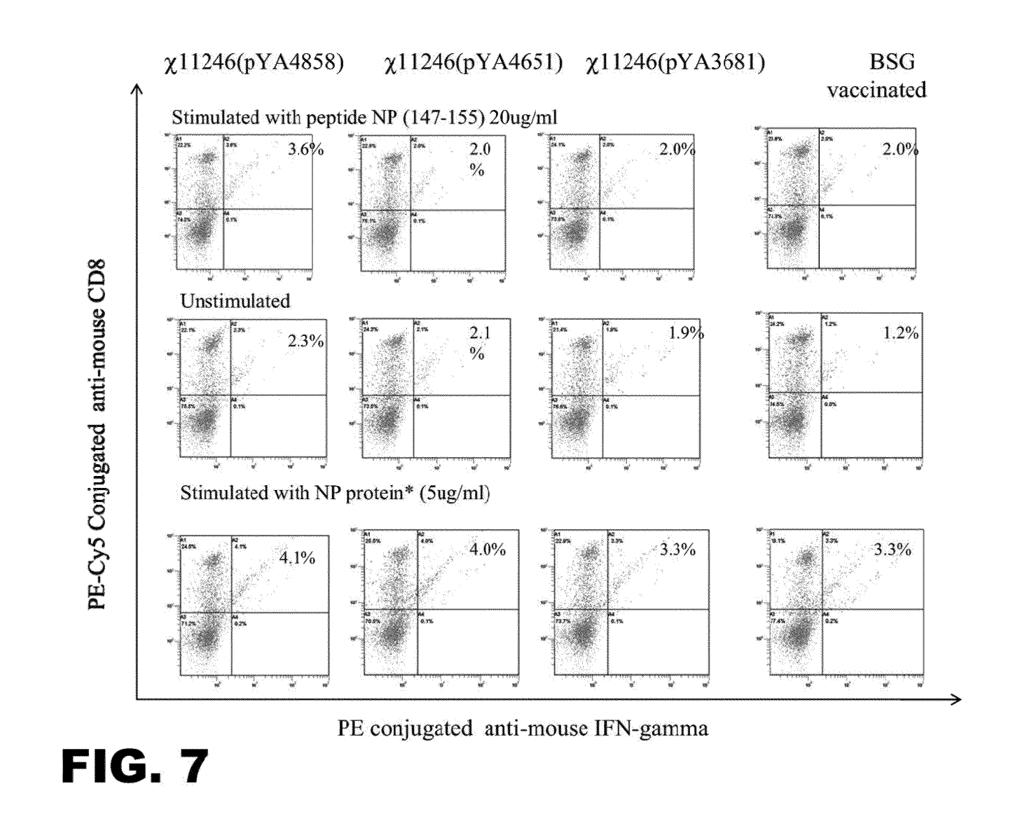

FIG. 7. Trial 2. Flow cytometric analysis of IFN-γ secreting CD8 T cells. Single cell suspensions were prepared from splenocytes from three mice per group four days after second booster vaccination and stimulated with NP (147-155) for 24 hrs and analyzed for the presence of IFN-γ secreting CD8 T cells. Data were derived form 10,000 events acquired from each sample. Numbers are percentages of IFN-γ secreting CD8 T cells.

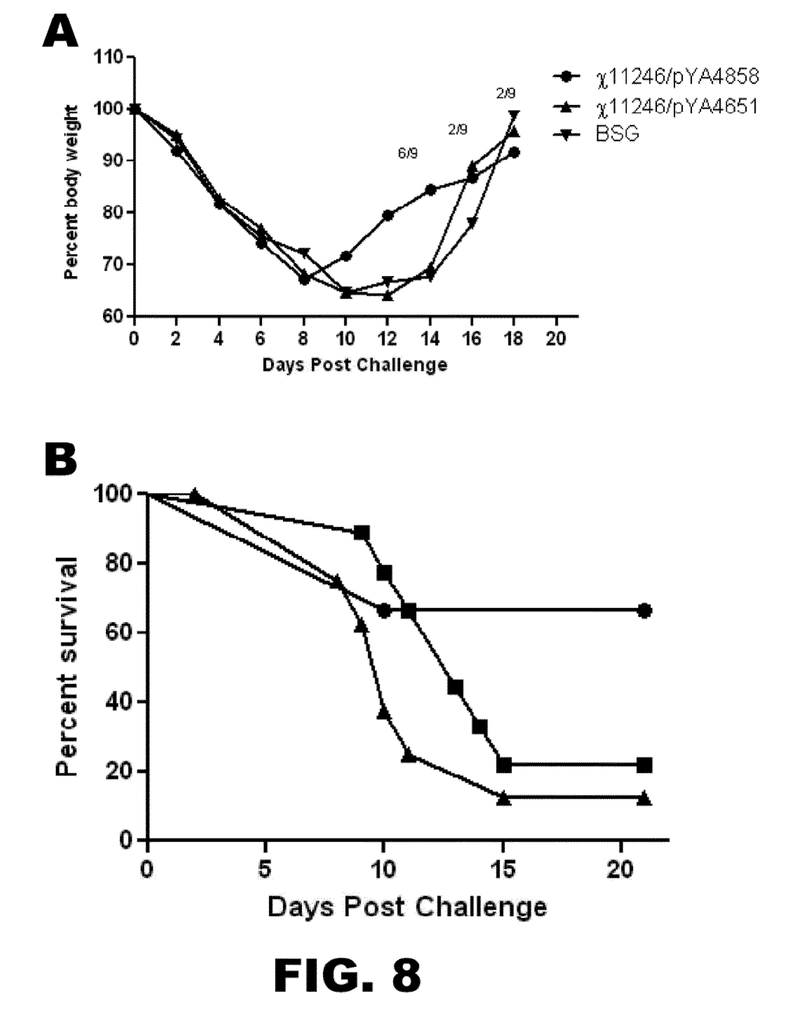

FIG. 8. Trial 2. Weight loss (A) and percent survival (B) of mice (n=5) orally immunized with two boosters after an intranasal challenge with 100 LD₅₀ of rWSN influenza virus at 5 weeks PPI.

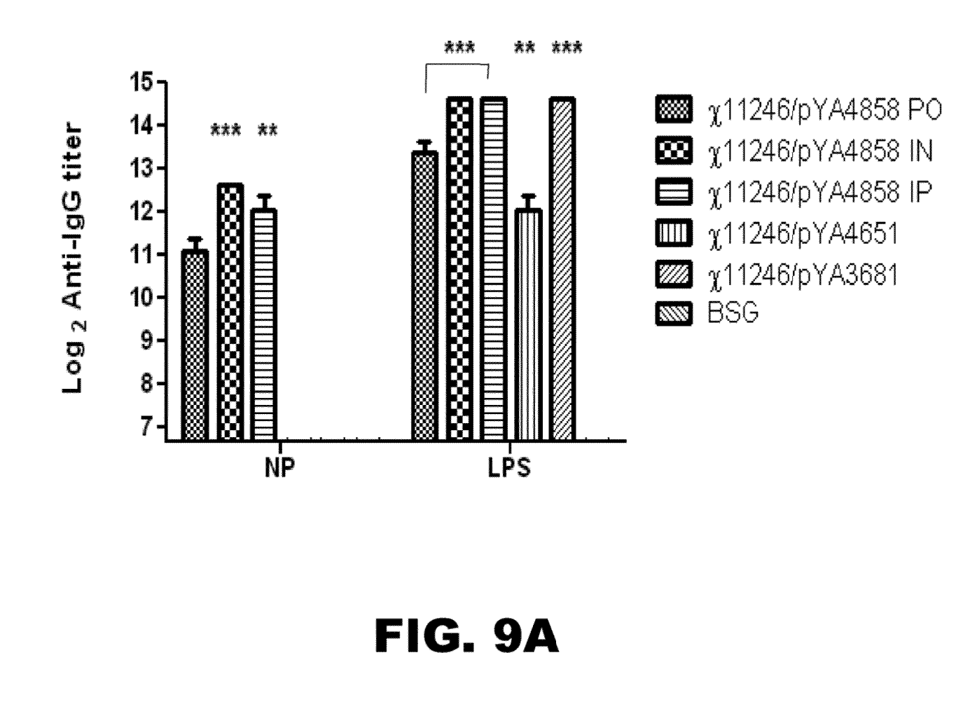

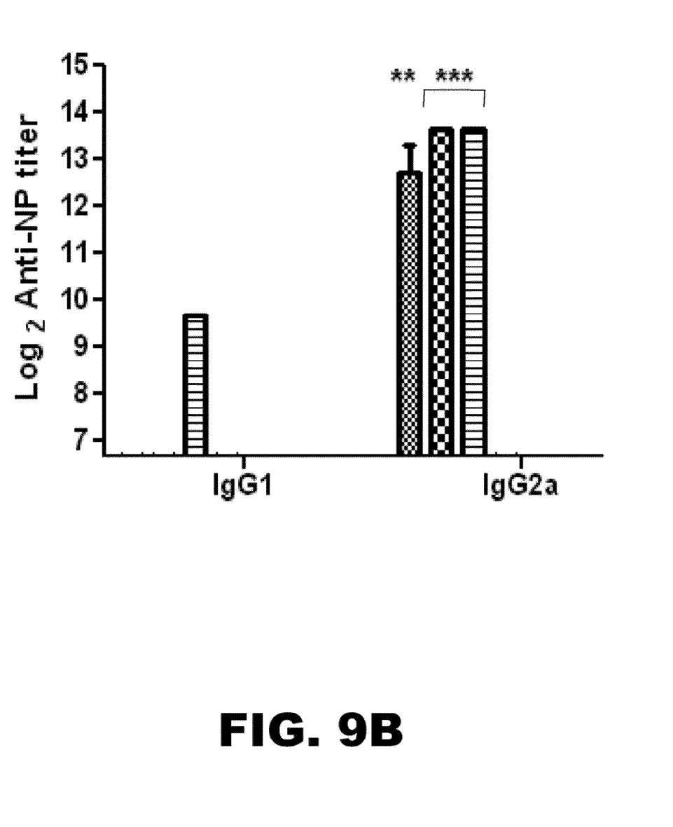

FIG. 9. Trial 3. Antibody titers detected by ELISA in mice immunized via oral (PO) intranasal (IN) or intraperitoneal (IP) routes, 6 weeks after three booster immunizations with the recombinant attenuated Salmonella strains χ11246(pYA4858) (SifA⁻) expressing influenza NP or BSG. (A). Induction of IgG titers against Influenza NP protein and purified S. Typhimurium LPS by ELISA. (B). Induction of IgG1 and IgG2a responses against influenza NP protein by ELISA. Pooled serum samples (n=12) from mice within a group were assayed and analyzed by two-way ANOVA followed by Bonferroni test. ***P<0.001.

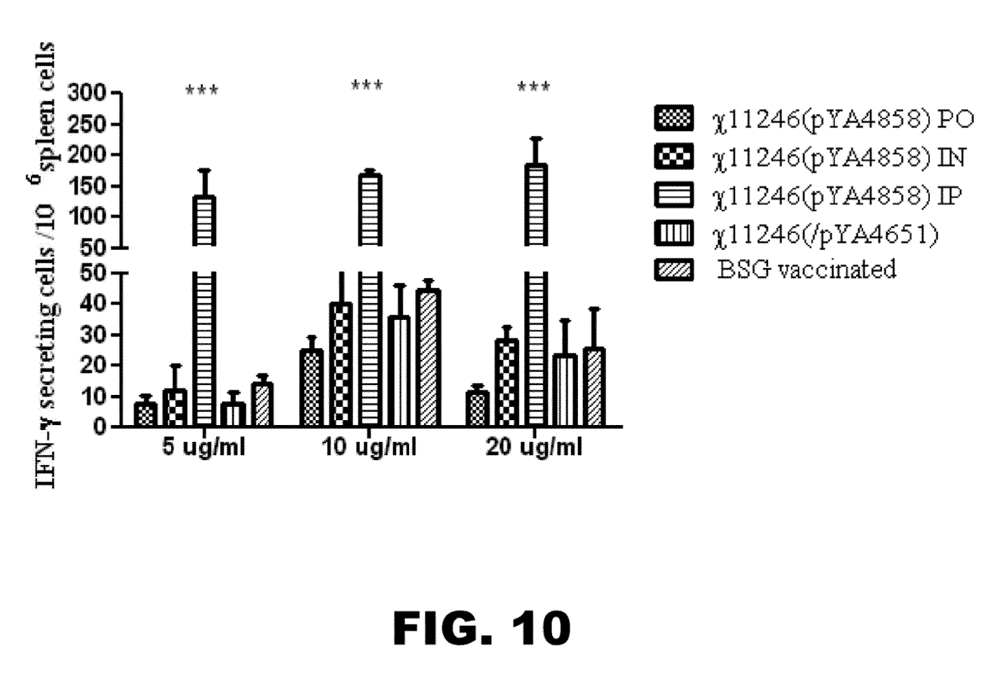

FIG. 10. Trial 3. ELISPOT analysis of IFN-γ production by NP₁₄₇₋₁₅₅ specific CD8⁺ T cells. Mice were boosted thrice with χ11246(pYA4858) (NP⁺) (SifA⁻) via PO, IN and IP routes. Splenocytes (n=3) from immunized mice were harvested at 8 weeks PPI and stimulated with NP₁₄₇₋₁₅₅ peptide for 48 h. Statistical analysis was performed by ANOVA followed by Tukey's method with 95% confidence interval. *** P<0.0001

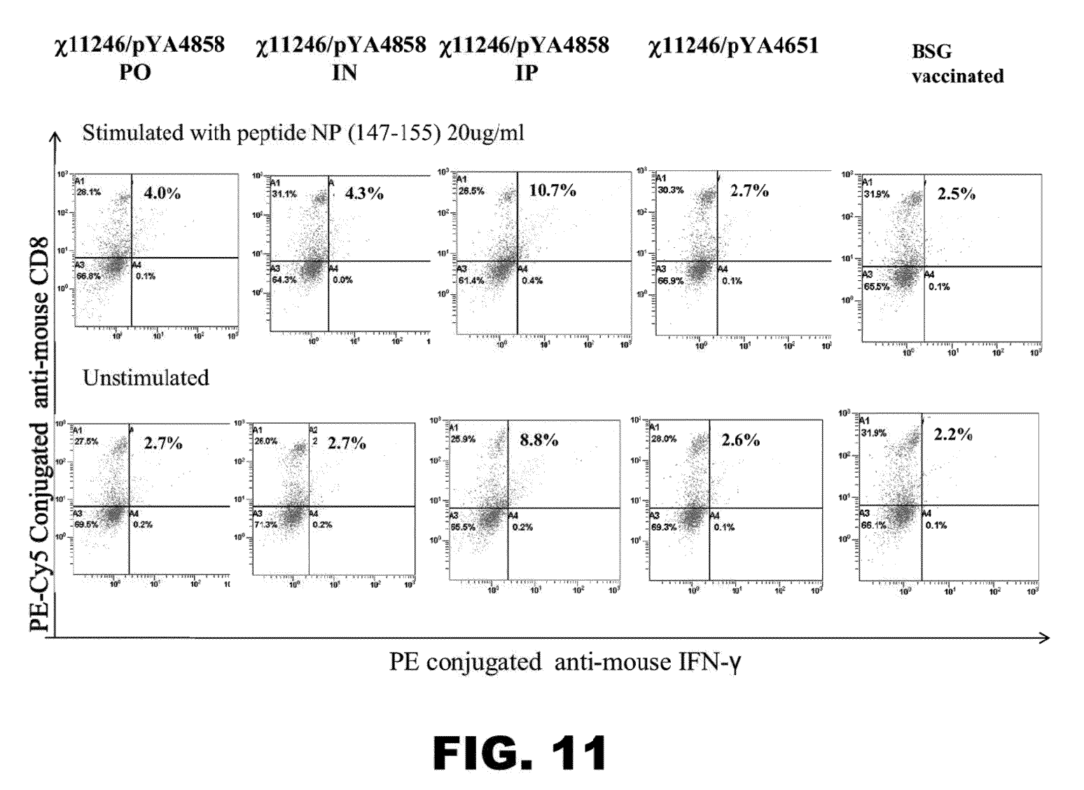

FIG. 11. Trial 3. Flow cytometric analysis of intracellular cytokine. Single cell suspensions were prepared from splenocytes from three mice per group four days after third booster vaccination and stimulated with NP (147-155) for 24 hrs and analyzed for the presence of IFN-γ secreting CD8 T cells. Data were derived from 10,000 events acquired from each sample. Numbers are percentages of IFN-γ secreting CD8 T cells and represent average from duplicate samples.

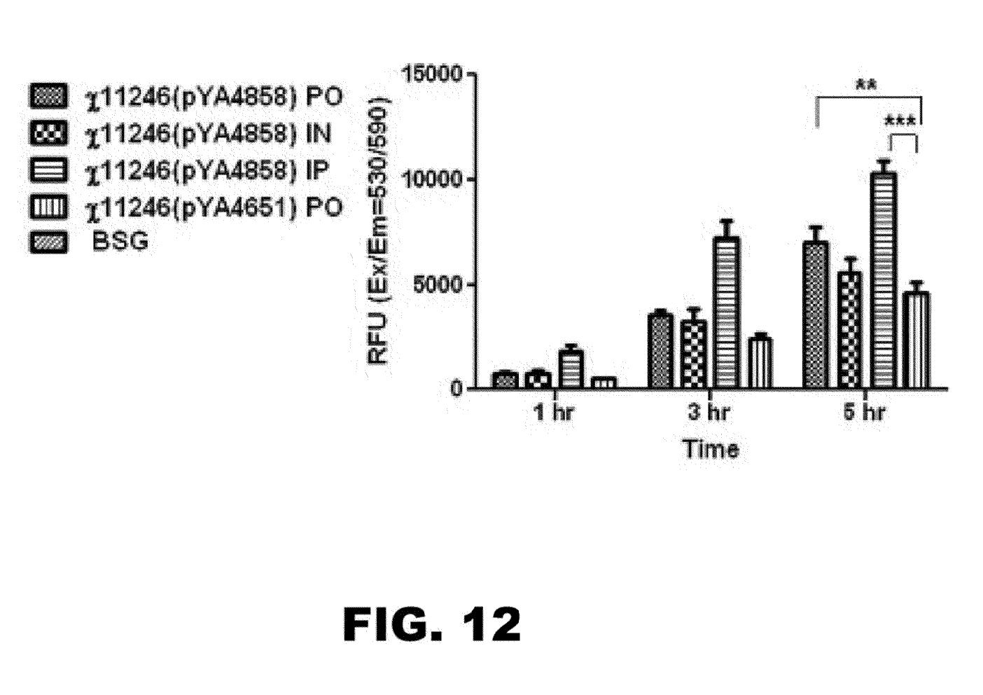

FIG. 12. Trial 3. Cell proliferation assay. Splenocytes (n=3) harvested from these mice were stimulated with NP₁₄₇₋₁₅₈ peptide (20 μg/ml) for 6 days and incubated with the Vision blue dye (Biovision). Plates were read at Ex 530 and Em 590 nm. Relative fluorescence units (RFUs) were calculated by subtracting background reading from unstimulated cells from the stimulated cells. Data were analyzed by two-way ANOVA followed by Bonferroni test. **P<0.05; ***P<0.01. PO=oral; IN=intranasal and IP=intraperitoneal.

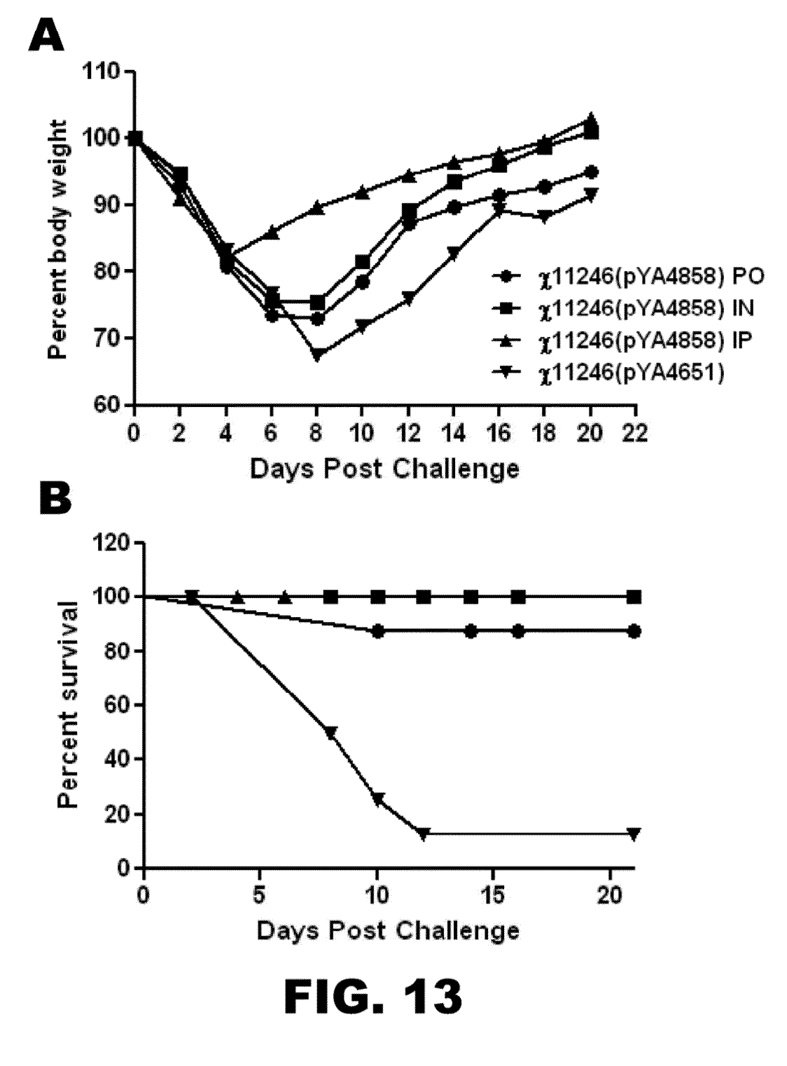

FIG. 13. Weight loss (A) and survival data (B) of mice after three boosters with χ11246(pYA4858) (NP⁺) (SifA⁻) via PO, IN and IP routes and χ11246(pYA4651)(Ply⁺)(SifA⁻) as a negative control at 8 weeks PPI. Weight loss (left) and Percent survival (right) of mice after three booster immunizations and an intranasal challenge with 100 LD₅₀ of rWSN influenza virus (n=8) at 8 weeks PPI.

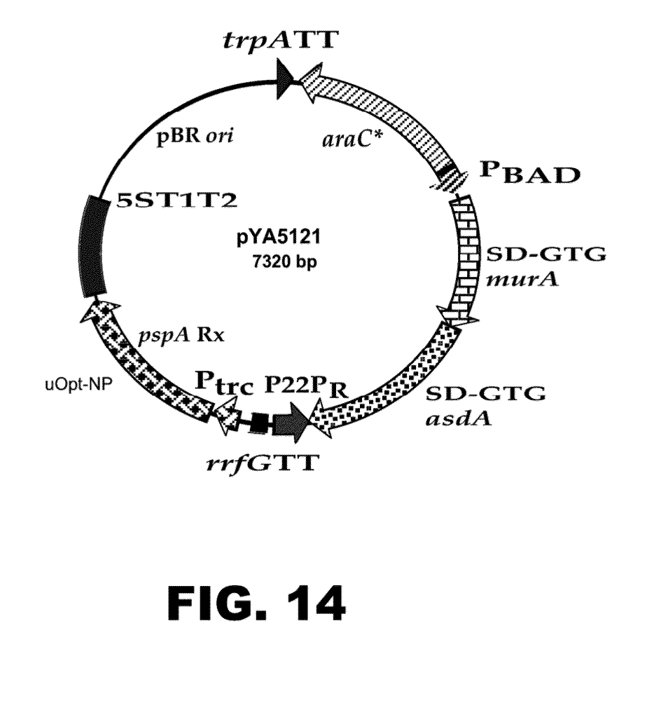

FIG. 14. Map of pYA5121 carrying codon-optimized NP gene (updated) in lysis vector pYA3681 for maximal expression in S. Typhimurium.

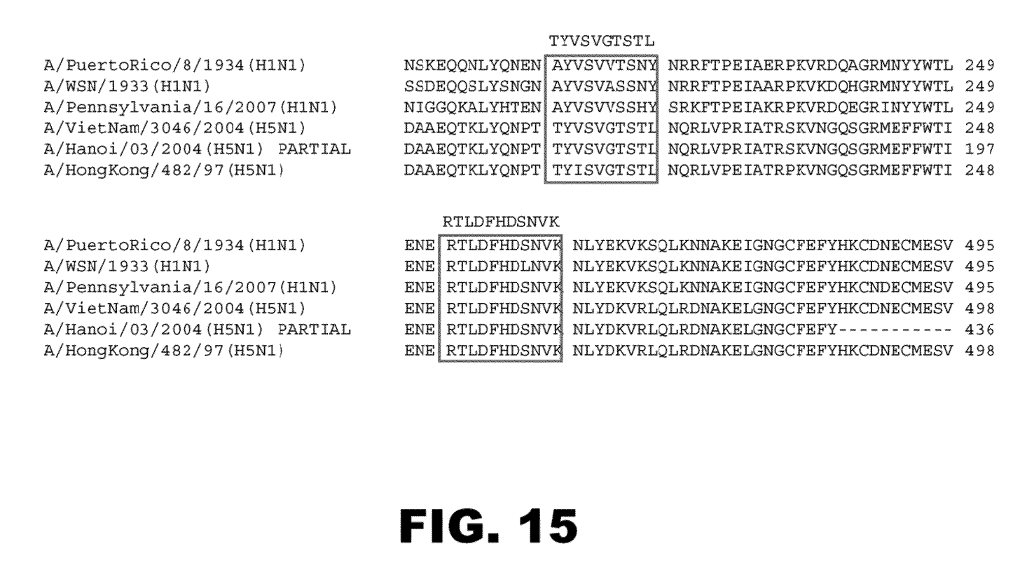

FIG. 15. Alignment of proposed HA T cell epitopes to Influenza strains of interest.

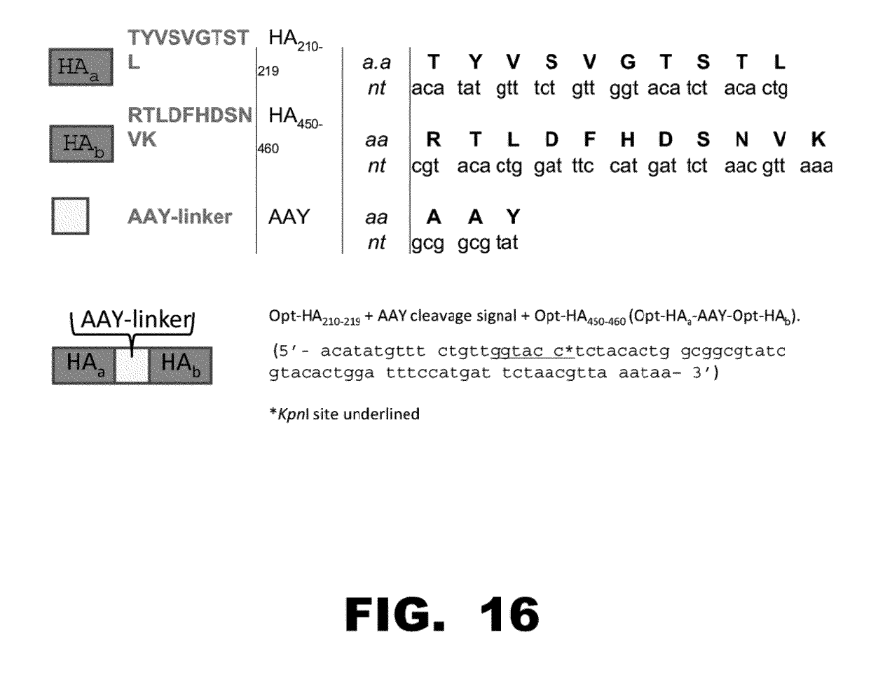

FIG. 16. Nucleotide (nt) sequence and structure of proposed HA T cell epitope tag (Opt-HAₐ-AAY-Opt-HA_{b}).

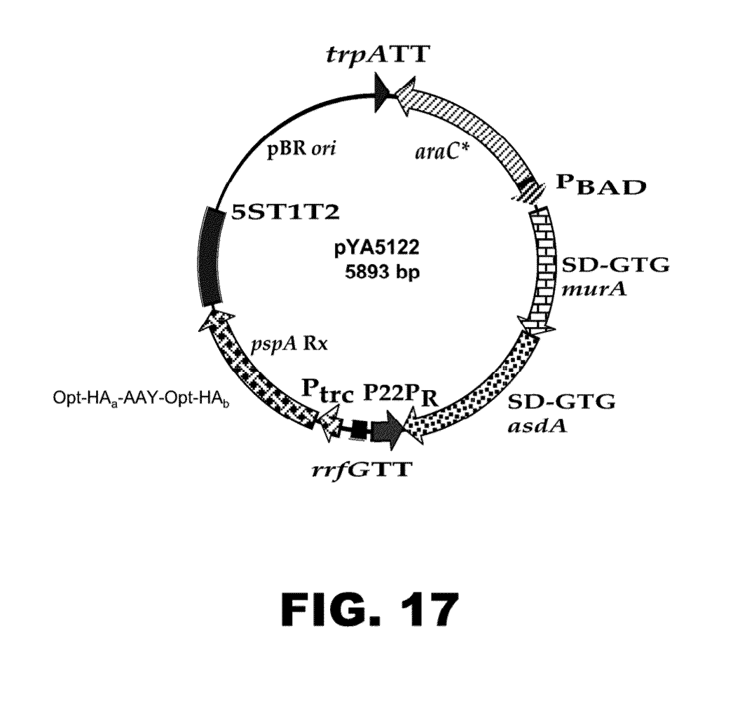

FIG. 17. Map of pYA5122 carrying P_{trc}-Opt-HAₐ-AAY-Opt-HA_{b} encoding sequence in lysis vector pYA3681 for maximal expression in S. Typhimurium serving as a HA-tag only control plasmid.

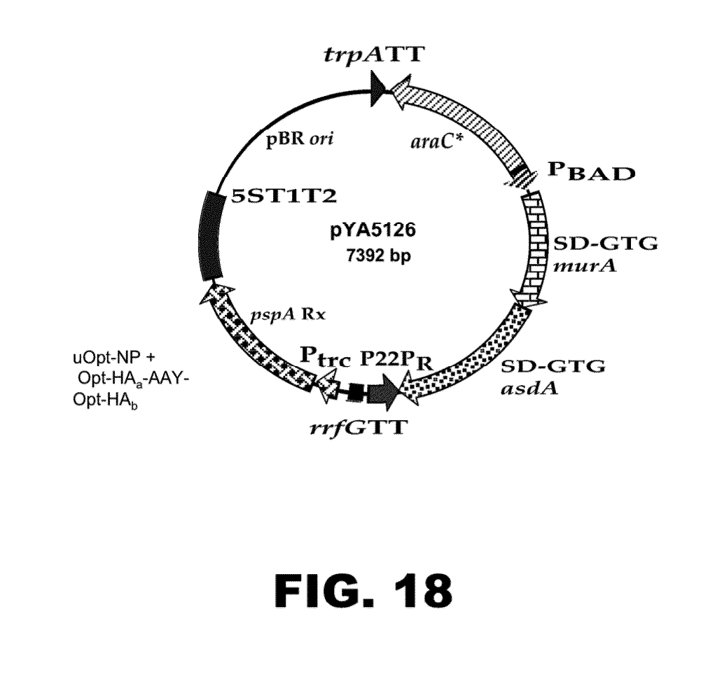

FIG. 18. Map of pYA5126 carrying codon-optimized NP gene (updated) with encoded C-terminal in-frame fused HA T cell epitope tag (Opt-HAₐ-AAY-Opt-HA_{b}) in lysis vector pYA3681 for maximal expression in S. Typhimurium.

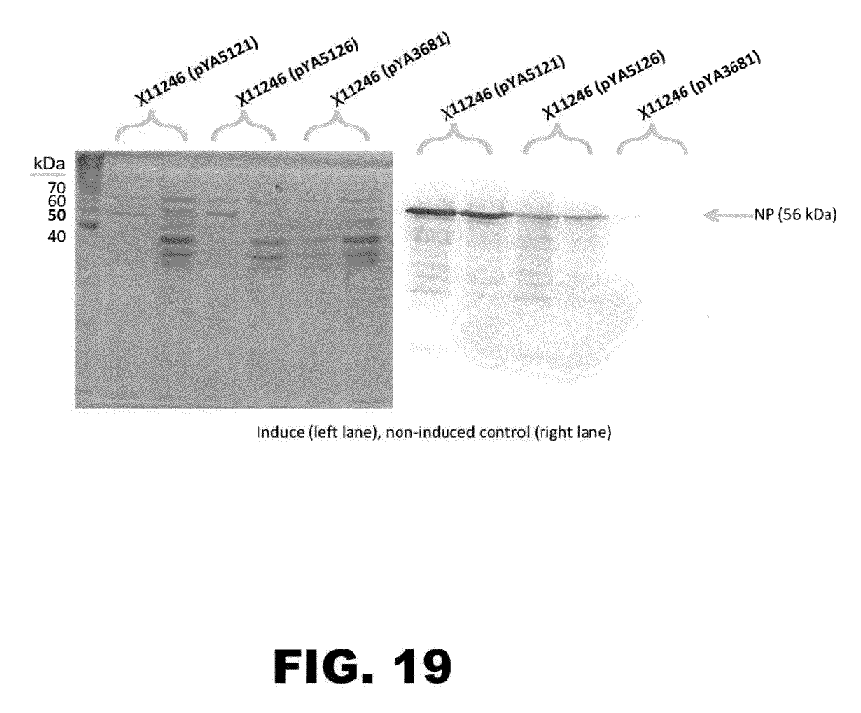

FIG. 19. SDS-PAGE and western blot analysis of cell lysates from χ11246 carrying pYA3681 (lysis vector control); pYA5121 (updated codon optimized NP), and pYA5126 (updated codon optimized NP+Opt-H_{Aa}-AAY-Opt-H_{Ab}).

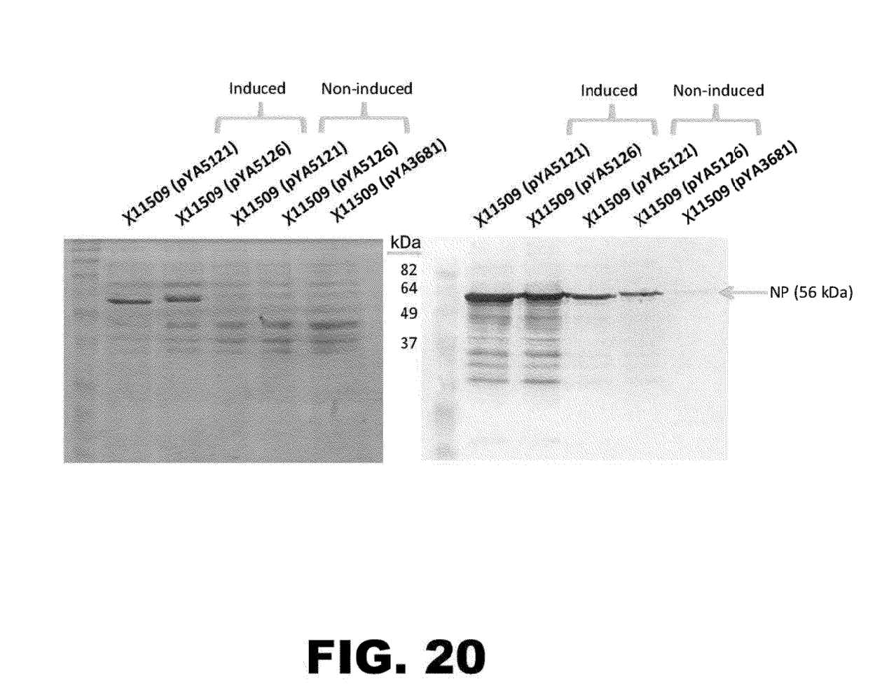

FIG. 20. SDS-PAGE and western blot analysis of cell lysates from χ11509 carrying pYA3681 (lysis vector control), pYA5121 (updated codon optimized NP), and pYA5126 (updated codon optimized NP+Opt-HAₐ-AAY-Opt-HA_{b}).

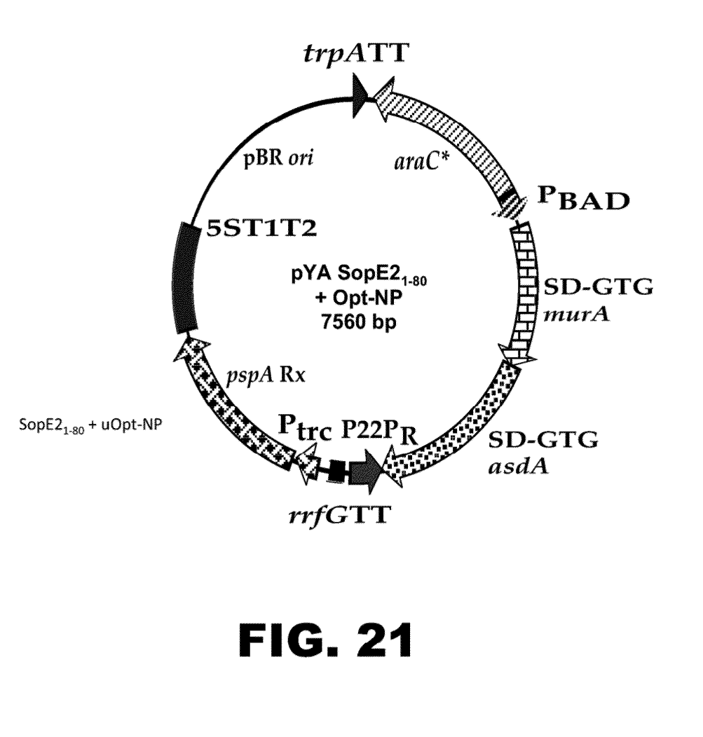

FIG. 21. Map of pYA SopE2₁₋₈₀+uOpt-NP carrying codon-optimized NP gene (updated) with encoded N-terminal in-frame fused SopE2 N-terminal 1-80 amino acids in lysis vector pYA3681 for maximal expression in S. Typhimurium.

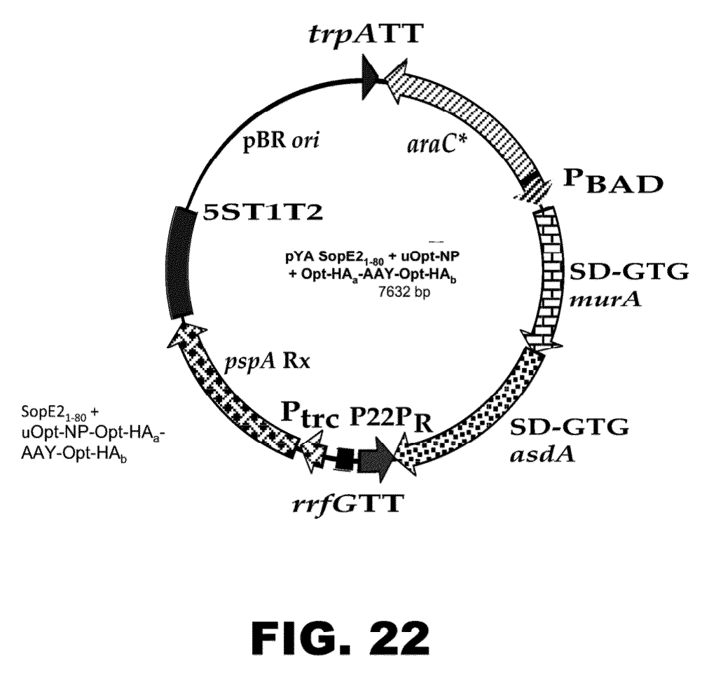

FIG. 22. Map of pYA SopE2₁₋₈₀+uOpt-NP+Opt-HAₐ-AAY-Opt-HA_{b} carrying codon-optimized NP gene (updated) with encoded N-terminal in-frame fused SopE2 N-terminal 1-80 amino acids and encoded C-terminal in-frame fused Opt-HAₐ-AAY-Opt-HA_{b} in lysis vector pYA3681 for maximal expression in S. Typhimurium.

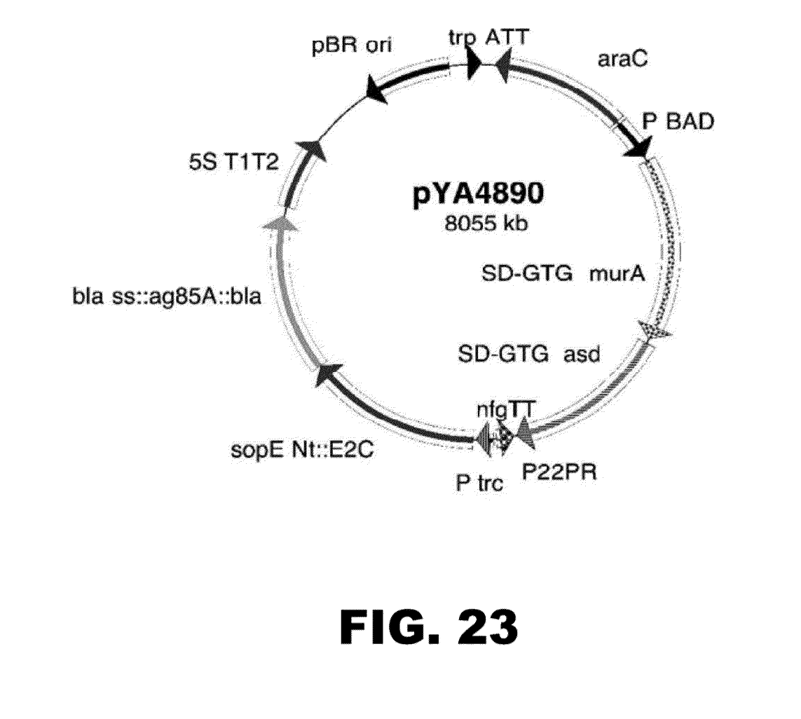

FIG. 23. Map of pYA4890 carrying two copies of the gene for ESAT-6 and one copy of the gene for CFP-10 fused to nucleotides encoding the first 80 amino acids from the N-terminus of Salmonella protein SopE and one copy of the gene for Ag85A in the lysis vector pYA3681.

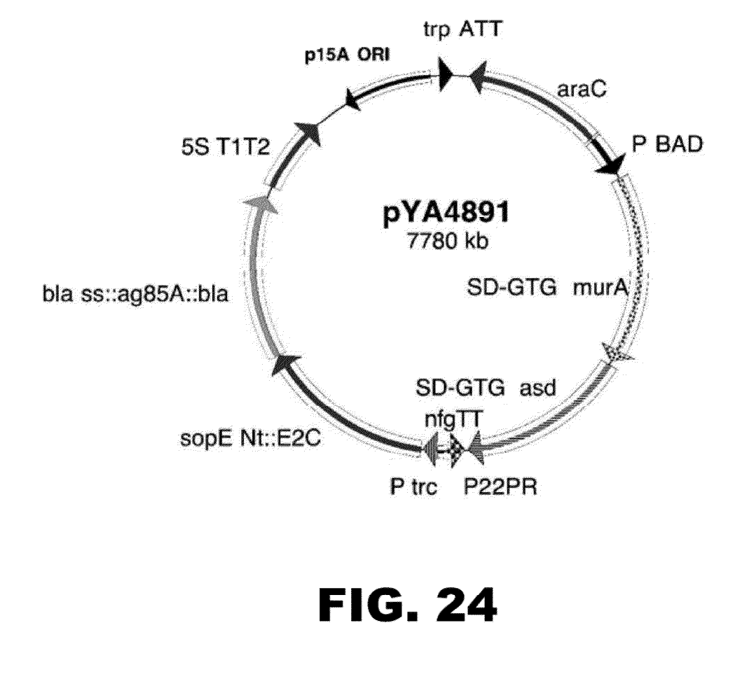

FIG. 24. Map of pYA4891 carrying two copies of the gene for ESAT-6 and one copy of the gene for CFP-10 fused to the nucleotides encoding the first 80 amino acids from the N-terminus of the Salmonella protein SopE and one copy of the gene for Ag85A in the lysis vector pYA4589 (a derivative of pYA3681 in which the p15A ori replaced the pBR ori of pYA3681).

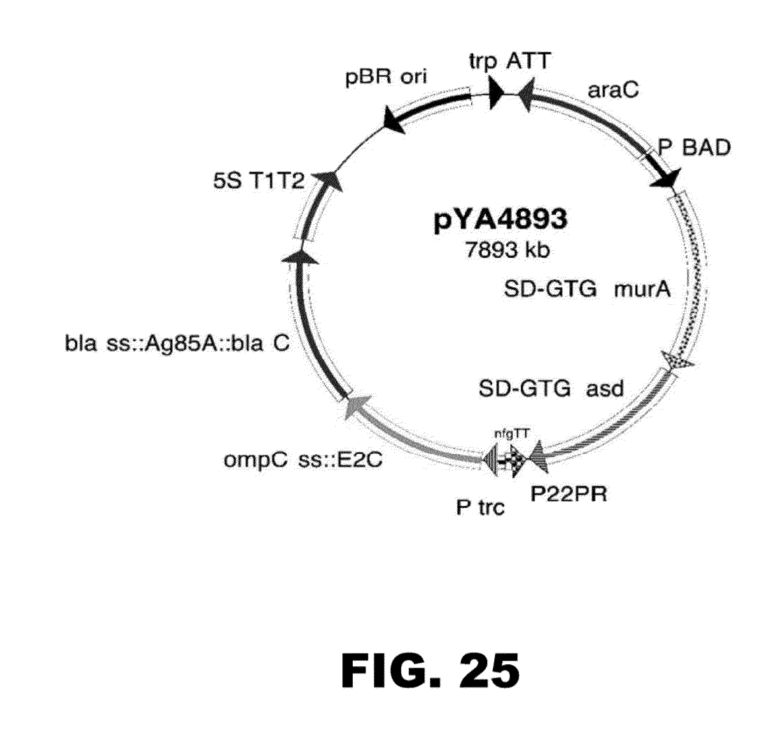

FIG. 25. Map of pYA4893 carrying two copies of the gene for ESAT-6 and one copy of the gene for CFP-10 fused with nucleotides encoding the signal sequence of the gene for Salmonella protein OmpC and one copy of the gene for Ag85A in the lysis vector pYA3681.

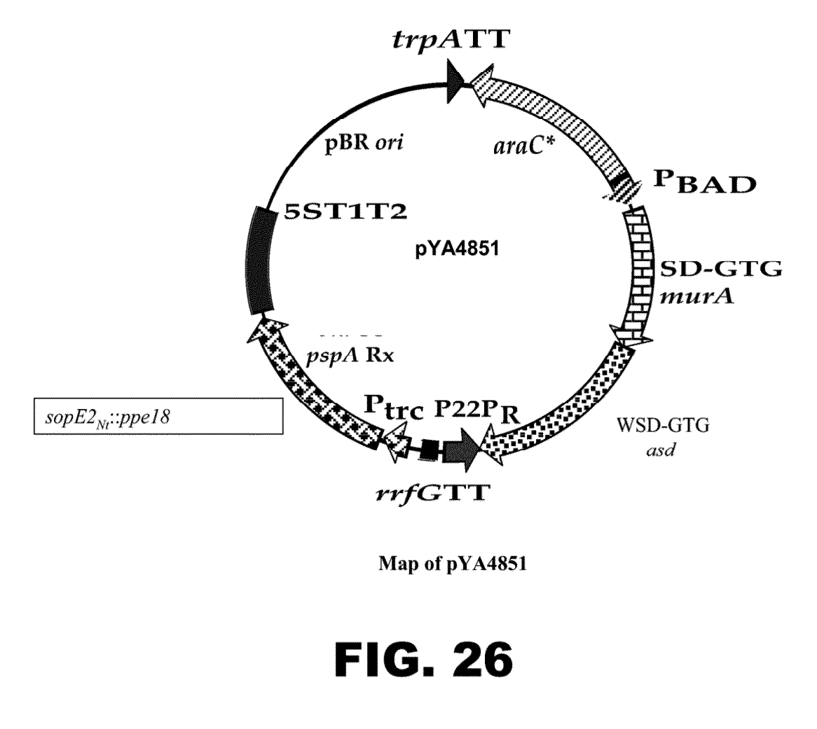

FIG. 26. Map of pYA4851 carrying the gene encoding Mtb39A fused to the nucleotides encoding the first 80 amino acids from the N-terminus of the Salmonella protein SopE2 in lysis vector pYA3681.

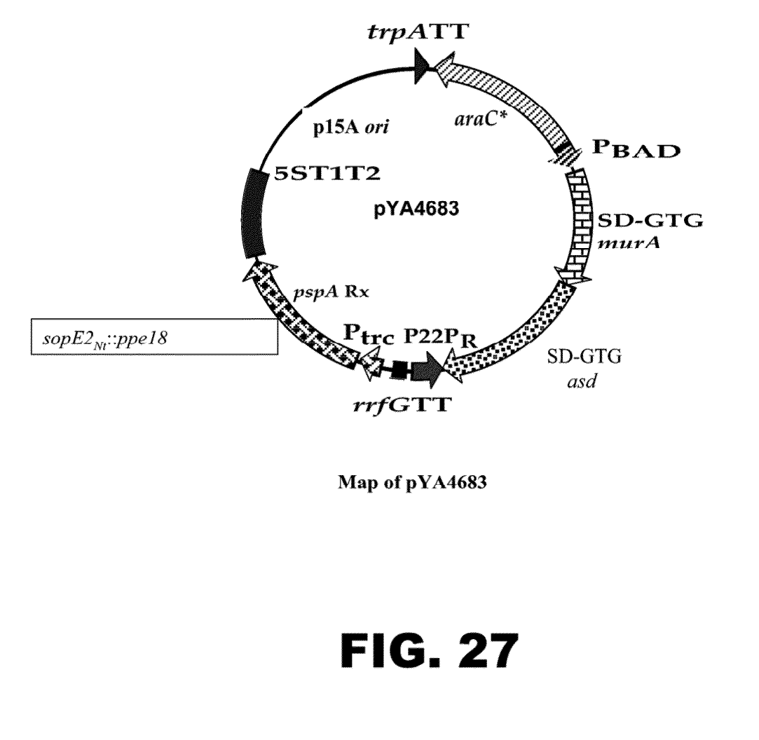

FIG. 27. Map of pYA4683 carrying the gene encoding Mtb39A fused to the nucleotides encoding the first 80 amino acids from the N-terminus of the Salmonella protein SopE2 in lysis vector pYA4589 (a derivative of pYA3681 in which the p15A on replaced the pBR on of pYA3681).

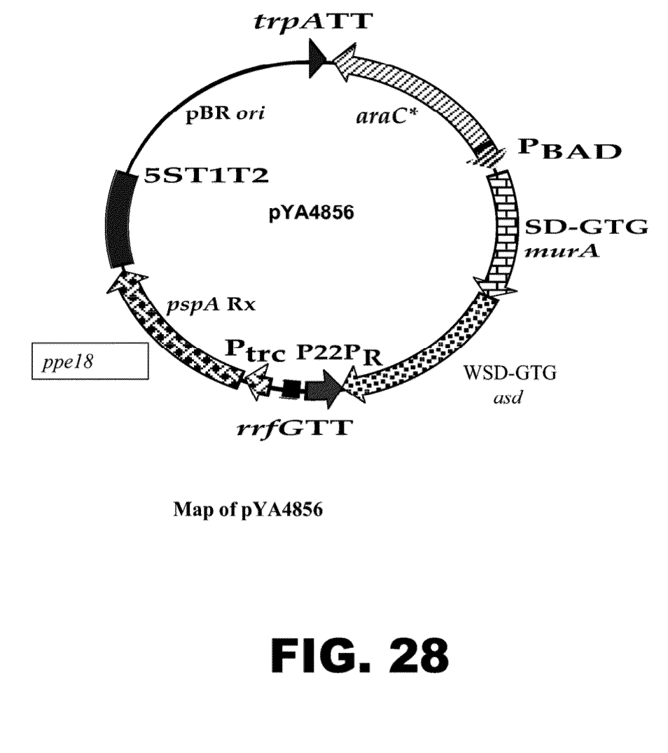

FIG. 28. Map of pYA4856 carrying the gene for Mtb39A in lysis vector pYA3681.

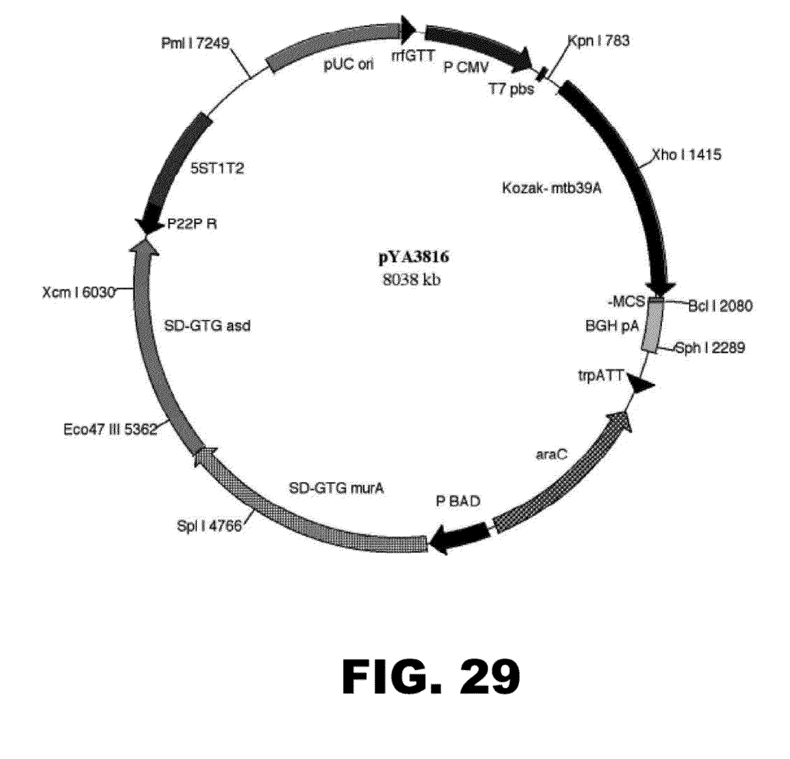

FIG. 29. Map of pYA3816 carrying the gene encoding Mtb39A in DNA vector pYA3650.

### DETAILED DESCRIPTION OF THE INVENTION

The present invention provides a recombinant bacterium that may be used to elicit an immune response from a host. In an exemplary embodiment, the immune response is a cellular immune response. Stated another way, the immune response is a Th1 T cell response. The invention also provides a vaccine comprising a recombinant bacterium of the invention, and methods of eliciting an immune response comprising administering a recombinant bacterium of the invention to a host.

### I. Recombinant Bacterium

A recombinant bacterium of the invention typically belongs to the Enterobaceteriaceae. The Enterobacteria family comprises species from the following genera: Alterococcus, Aquamonas, Aranicola, Arsenophonus, Brenneria, Budvicia, Buttiauxella, Candidatus Phlomobacter, Cedeceae, Citrobacter, Edwardsiella, Enterobacter, Erwinia, Escherichia, Ewingella, Hafnia, Klebsiella, Kluyvera, Leclercia, Leminorella, Moellerella, Morganella, Obesumbacterium, Pantoea, Pectobacterium, Photorhabdus, Plesiomonas, Pragia, Proteus, Providencia, Rahnella, Raoultella, Salmonella, Samsonia, Serratia, Shigella, Sodalis, Tatumella, Trabulsiella, Wigglesworthia, Xenorhbdus, Yersinia, Yokenella. In certain embodiments, the recombinant bacterium is typically a pathogenic species of the Enterobaceteriaceae. Due to their clinical significance, Escherichia coli, Shigella, Edwardsiella, Salmonella, Citrobacter, Klebsiella, Enterobacter, Serratia, Proteus, Morganella, Providencia and Yersinia are considered to be particularly useful. In other embodiments, the recombinant bacterium may be a species or strain commonly used for a vaccine.

Some embodiments of the instant invention comprise a species or subspecies of the Salmonella genera. For instance, the recombinant bacterium may be a Salmonella enterica serovar. In an exemplary embodiment, a bacterium of the invention may be derived from S. typhimurium, S. typhi, S. paratyphi, S. gallinarum, S. enteritidis, S. choleraesius, S. arizona, or S. dublin.

A recombinant bacterium of the invention derived from Salmonella may be particularly suited to use as a vaccine. Infection of a host with a Salmonella strain typically leads to colonization of the gut-associated lymphoid tissue (GALT) or Peyer's patches, which leads to the induction of a generalized mucosal immune response to the recombinant bacterium. Further penetration of the bacterium into the mesenteric lymph nodes, liver and spleen may augment the induction of systemic and cellular immune responses directed against the bacterium. Thus the use of recombinant Salmonella can stimulate all three branches of the immune system, which is particularly important for immunizing against infectious disease agents that colonize on and/or invade through mucosal surfaces.

A bacterium of the invention may comprise one or more mutations as detailed below. In particular, a bacterium may comprise one or more mutations to allow endosomal escape (section (a) below), one or more mutations to induce lysis of the bacterium (section (b) below), one or more mutations to express a nucleic acid encoding an antigen (section (c) below), one or more mutations to attenuate the bacterium (section (d) below), and/or one or more mutations to enhance the performance of the bacterium as a vaccine (section (e) below).

### (a) Endosomal Escape

A recombinant bacterium of the invention may be capable of escaping the endosomal compartment of the host cell. Escape typically facilitates delivery of an antigen to the cytosol of the host cell. A recombinant bacterium may escape from the endosome immediately after invasion of the host cell, or alternatively, may delay escape. Methods of detecting escape from the endosomal compartment are well known in the art, and may include microscopic analysis.

In one embodiment, a recombinant bacterium capable of escaping the endosomal compartment comprises a mutation that alters the functioning of SifA. For instance, sifA may be mutated so that the function of the protein encoded by sifA is altered. Non-limiting examples include a mutation that deletes sifA (ΔsifA). Such a mutation allows escape from the endosome upon host-cell invasion. Another example is a ΔP_{sifA}::TT araC P_{BAD} sifA mutation, which allows delayed escape. Since arabinose is absent in host tissues the expression of the sifA gene ceases and no SifA protein is synthesized such that the amount decreases with each round of bacterial cell division thereby allowing escape from the endosome. Similar delayed escape mutations may be constructed using other regulatable promoters, such as from the xylose or rhamnose regulatory systems.

In another embodiment, a recombinant bacterium capable of escaping the endosomal compartment may comprise a mutation that causes the expression of nucleic acid sequences such as tlyC or pld from Rickettsiae prowazekii. The expression may be regulated by an inducible promoter. For instance, the bacterium may comprise an araC P_{BAD} tlyC or an araC P_{BAD} pld mutation. In some embodiments, a bacterium may comprise a sifA mutation and a mutation that causes the expression of tlyC or pld.

### (b) Lysis

In another embodiment, a recombinant bacterium of the invention is capable of regulated lysis. Lysis of the bacterium within the host cell may release a bolus of antigen. Lysis also provides a means of biocontainment. For additional biocontainment mechanisms, see section (e) below.

In some embodiments, a recombinant bacterium capable of regulated lysis may comprise a mutation in a required constituent of the peptidoglycan layer of the bacterial cell wall. For instance, the bacterium may comprise a mutation in a nucleic acid sequence encoding a protein involved in muramic acid synthesis, such as murA. It is not possible to alter murA by deletion, however, because a ΔmurA mutation is lethal and can not be isolated. This is because the missing nutrient required for viability is a phosphorylated muramic acid that cannot be exogenously supplied since enteric bacteria cannot internalize it. Consequently, the murA nucleic acid sequence may be altered to make expression of murA dependent on a nutrient (e.g., arabinose) that can be supplied during the growth of the bacterium. For example, the alteration may comprise a ΔP_{murA}::TT araC P_{BAD} murA deletion-insertion mutation. During in vitro growth of the bacterium, this type of mutation makes synthesis of muramic acid dependent on the presence of arabinose in the growth medium. During growth of the bacterium in a host, however, arabinose is absent. Consequently, the bacterium is non-viable and/or avirulent in a host unless the bacterium further comprises at least one extrachromosomal vector comprising a nucleic acid sequence, that when expressed, substantially functions as murA. Recombinant bacteria with a ΔP_{murA}:TT araC P_{BAD} murA deletion-insertion mutation grown in the presence of arabinose exhibit effective colonization of effector lymphoid tissues after oral vaccination prior to cell death due to cell wall-less lysing.

Similarly, in various embodiments a recombinant bacterium may comprise the araC P_{BAD} c2 cassette inserted into the asdA nucleic acid sequence that encodes aspartate semialdehyde dehydrogenase, a necessary enzyme for DAP synthesis, a required component of the peptidoglycan layer of the bacterial cell wall. The chromosomal asdA nucleic acid sequence is typically inactivated to enable use of plasmid vectors encoding the wild-type asdA nucleic acid sequence in the balanced-lethal host-vector system. This allows stable maintenance of plasmids in vivo in the absence of any drug resistance attributes that are not permissible in live bacterial vaccines.

In one embodiment, ΔasdA27::TT araC P_{BAD} c2 has an improved SD sequence and a codon optimized c2 nucleic acid sequence. The C2 repressor synthesized in the presence of arabinose is used to repress nucleic acid sequence expression from P22 P_{R} and P_{L} promoters. In another embodiment, ΔasdA27::TT araC P_{BAD} c2 has the 1104 base-pair asdA nucleic acid sequence deleted (1 to 1104, but not including the TAG stop codon) and the 1989 base-pair fragment containing T4 ipIII TT araC P_{BAD} c2 inserted. The c2 nucleic acid sequence in ΔasdA27::TT araC P_{BAD} c2 has a SD sequence that was optimized to TAAGGAGGT. It also has an improved P_{BAD} promoter such that the −10 sequence is improved from TACTGT to TATAAT. Furthermore, it has a codon optimized c2 nucleic acid sequence, in which the second codon was modified from AAT to AAA.

In exemplary embodiments, the bacterium may comprise a mutation in the murA nucleic acid sequence encoding the first enzyme in muramic acid synthesis and the asdA nucleic acid sequence essential for DAP synthesis. By way of non-limiting example, these embodiments may comprise the chromosomal deletion-insertion mutations ΔasdA19::TT araC P_{BAD} c2 or ΔasdA27::TT araC P_{BAD} c2 and ΔP_{murA7}::TT araC P_{BAD} murA or ΔP_{murA12}::TT araC P_{BAD} murA or ΔP_{murA25}::TT araC P_{BAD} murA. This host-vector grows in LB broth with 0.1% L-arabinose, but is unable to grow in or on media devoid of arabinose since it undergoes cell wall-less death by lysis.

Bacterium that comprise these mutations also comprise a plasmid that contains a nucleic acid sequence that substitutes for murA and asdA. This allows the bacterium to grow in permissive environments, e.g. when arabinose is present. For instance plasmid vector pYA3681 contains the murA nucleic acid sequence (with altered start codon sequences to decrease translation efficiency) under the control of an araC P_{BAD} promoter. The second nucleic acid sequence under the direction of this promoter is the asdA nucleic acid sequence (with altered start codon sequences to decrease translation efficiency). The P22 P_{R} promoter is in the anti-sense direction of both the asdA nucleic acid sequence and the murA nucleic acid sequence. The P22 P_{R} is repressed by the C2 repressor made during growth of the strain in media with arabinose (due to the ΔasdA::TT araC P_{BAD} c2 deletion-insertion). However C2 concentration decreases due to cell division in vivo to cause P_{R} directed synthesis of anti-sense mRNA to further block translation of asdA and murA mRNA. The araC P_{BAD} sequence is also not from E. coli B/r as originally described but represents a sequence derived from E. coli K-12 strain χ289 with tighter control and less leakiness in the absence of arabinose. In the preferred embodiment, transcription terminators (TT) flank all of the domains for controlled lysis, replication, and expression so that expression in one domain does not affect the activities of another domain. As a safety feature, the plasmid asdA nucleic acid sequence does not replace the chromosomal asdA mutation since they have a deleted sequence in common. Additionally, the E. coli murA nucleic acid sequence was used in the plasmid instead of using the Salmonella murA nucleic acid sequence. In addition to being fully attenuated, this construction exhibits complete biological containment. This property enhances vaccine safety and minimizes the potential for vaccination of individuals not intended for vaccination.

One of skill in the art will recognize that other nutrients besides arabinose may be used in the above mutations. By way of non-limiting example, xylose, mannose, and rhamnose regulatory systems may also be used.

In some embodiments of the invention, the recombinant bacterium may further comprise araBAD and araE mutations to preclude breakdown and leakage of internalized arabinose such that asdA and murA nucleic acid sequence expression continues for a cell division or two after oral immunization into an environment that is devoid of external arabinose. Additionally, a bacterium may comprise a mutation in a protein involved in GDP-fucose synthesis to preclude formation of colonic acid. Non-limiting examples of such a mutation include Δ(gmd-fcl). A bacterium may also comprise a mutation like ΔrelA that uncouples cell wall-less death from dependence on protein synthesis.

Lysis of the bacterium will typically release lipid A, an endotoxin. So, a bacterium of the invention may comprise a mutation that reduces the toxicity of lipid A. Non-limiting examples may include a mutation that causes synthesis of the mono-phosphoryl lipid A. This form of lipid A is non-toxic, but still serves as an adjuvant agonist.

Alternatively, a recombinant bacterium of the invention may comprise a lysis system disclosed in Kong et al., (2008) PNAS 105:9361 or US Patent Publication No. 2006/0140975, each of which is hereby incorporated by reference in its entirety.

### (c) Antigen Synthesis

A recombinant bacterium of the invention may express or deliver one or more nucleic acids that encode one or more antigens. For instance, in one embodiment, a recombinant bacterium may be capable of the regulated expression of a nucleic acid sequence encoding an antigen. In another embodiment, a recombinant bacterium may comprise a nucleic acid vaccine vector. In yet another embodiment, a recombinant bacterium may comprise an eight unit viral cassette. Each of the above embodiments is described in more detail below. Other means of expressing or delivering one or more nucleic acids that encode one or more antigens are known in the art.

In one embodiment, the antigen is an Eimeria antigen. For instance, non-limiting examples of Eimeria antigens may include EASZ240, EAMZ250, TA4, EtMIC2, or SO7.

In another embodiment, the antigen may be a viral antigen. For example, the antigen may be an influenza antigen. Non-limiting examples of influenza antigens may include M2e, nucleoprotein (NP), hemagglutinin (HA), and neuraminidase (NA). Antigens may be fused to a protein to enhance antigen processing within a host cell. For instance, an antigen may be fused with SopE, SptP, woodchuck hepatitis core antigen, or HBV core antigen. Additional examples of antigens may be found in sections i., ii., and iii. below and in the Examples.

In another embodiment, the antigen is an antigen from M. tuberculosis. For instance, non-limiting examples of M. tuberculosis antigens may include ESAT-6, CFP-10, Ag85A, Ag85B, Ag85C, Mtb39A, FAP (fibronectin attachment protein), Tb15.3, RfpA and RfpB or any other antigens that would induce a T-cell immune response.

Antigens of the invention may be delivered via a type 2 or a type 3 secretion system, by a regulated delayed lysis in vivo system, by endosomal escape, or a combination thereof. For more details, see the Examples.

The expression level of the nucleic acid sequence encoding the antigen may be modified using methods known in the art, and as described for optimizing expression of the repressor below.

i. Regulated Expression

The present invention encompasses a recombinant bacterium capable of the regulated expression of at least one nucleic acid sequence encoding an antigen of interest. Generally speaking, such a bacterium comprises a nucleic acid sequence encoding a repressor and a vector. Each is discussed in more detail below.

### A. Nucleic Acid Sequence Encoding a Repressor

A recombinant bacterium of the invention that is capable of the regulated expression of at least one nucleic acid sequence encoding an antigen comprises, in part, at least one nucleic acid sequence encoding a repressor. The nucleic acid may be chromosomally integrated. In other embodiments, the nucleic acid may be on an extrachromosomal vector. Typically, the nucleic acid sequence encoding a repressor is operably linked to a regulatable promoter. The nucleic acid sequence encoding a repressor and/or the promoter may be modified from the wild-type nucleic acid sequence so as to optimize the expression level of the nucleic acid sequence encoding the repressor.

Methods of chromosomally integrating a nucleic acid sequence encoding a repressor operably-linked to a regulatable promoter are known in the art and detailed in the examples. Generally speaking, the nucleic acid sequence encoding a repressor should not be integrated into a locus that disrupts colonization of the host by the recombinant bacterium, or attenuates the bacterium. In one embodiment, the nucleic acid sequence encoding a repressor may be integrated into the relA nucleic acid sequence. In another embodiment, the nucleic acid sequence encoding a repressor may be integrated into the endA nucleic acid sequence.

In some embodiments, at least one nucleic acid sequence encoding a repressor is chromosomally integrated. In other embodiments, at least two, or at least three nucleic acid sequences encoding repressors may be chromosomally integrated into the recombinant bacterium. If there is more than one nucleic acid sequence encoding a repressor, each nucleic acid sequence encoding a repressor may be operably linked to a regulatable promoter, such that each promoter is regulated by the same compound or condition. Alternatively, each nucleic acid sequence encoding a repressor may be operably linked to a regulatable promoter, each of which is regulated by a different compound or condition.

### 1. Repressor

As used herein, “repressor” refers to a biomolecule that represses transcription from one or more promoters. Generally speaking, a suitable repressor of the invention is synthesized in high enough quantities during the in vitro growth of the bacterial strain to repress the transcription of the nucleic acid sequence encoding an antigen of interest on the vector, as detailed below, and not impede the in vitro growth of the strain. Additionally, a suitable repressor will generally be substantially stable, i.e. not subject to proteolytic breakdown. Furthermore, a suitable repressor will be diluted by about half at every cell division after expression of the repressor ceases, such as in a non-permissive environment (e.g. an animal or human host).

The choice of a repressor depends, in part, on the species of the recombinant bacterium used. For instance, the repressor is usually not derived from the same species of bacteria as the recombinant bacterium. For instance, the repressor may be derived from E. coli if the recombinant bacterium is from the genus Salmonella. Alternatively, the repressor may be from a bacteriophage.

Suitable repressors are known in the art, and may include, for instance, LacI of E. coli, C2 encoded by bacteriophage P22, or C1 encoded by bacteriophage λ. Other suitable repressors may be repressors known to regulate the expression of a regulatable nucleic acid sequence, such as nucleic acid sequences involved in the uptake and utilization of sugars. In one embodiment, the repressor is LacI. In another embodiment, the repressor is C2. In yet another embodiment, the repressor is C1.

### 2. Regulatable Promoter

The chromosomally integrated nucleic acid sequence encoding a repressor is operably linked to a regulatable promoter. The term “promoter”, as used herein, may mean a synthetic or naturally-derived molecule that is capable of conferring, activating or enhancing expression of a nucleic acid. A promoter may comprise one or more specific transcriptional regulatory sequences to further enhance expression and/or to alter the spatial expression and/or temporal expression of a nucleic acid. The term “operably linked,” as used herein, means that expression of a nucleic acid sequence is under the control of a promoter with which it is spatially connected. A promoter may be positioned 5′ (upstream) of the nucleic acid sequence under its control. The distance between the promoter and a nucleic acid sequence to be expressed may be approximately the same as the distance between that promoter and the native nucleic acid sequence it controls. As is known in the art, variation in this distance may be accommodated without loss of promoter function.

The regulated promoter used herein generally allows transcription of the nucleic acid sequence encoding a repressor while in a permissive environment (i.e., in vitro growth), but ceases transcription of the nucleic acid sequence encoding a repressor while in a non-permissive environment (i.e., during growth of the bacterium in an animal or human host). For instance, the promoter may be sensitive to a physical or chemical difference between the permissive and non-permissive environment. Suitable examples of such regulatable promoters are known in the art.

In some embodiments, the promoter may be responsive to the level of arabinose in the environment. Generally speaking, arabinose may be present during the in vitro growth of a bacterium, while typically absent from host tissue. In one embodiment, the promoter is derived from an araC-P_{BAD} system. The araC-P_{BAD} system is a tightly regulated expression system, which has been shown to work as a strong promoter induced by the addition of low levels of arabinose (5). The araC-araBAD promoter is a bidirectional promoter controlling expression of the araBAD nucleic acid sequences in one direction, and the araC nucleic acid sequence in the other direction. For convenience, the portion of the araC-araBAD promoter that mediates expression of the araBAD nucleic acid sequences, and which is controlled by the araC nucleic acid sequence product, is referred to herein as P_{BAD}. For use as described herein, a cassette with the araC nucleic acid sequence and the araC-araBAD promoter may be used. This cassette is referred to herein as araC-P_{BAD}. The AraC protein is both a positive and negative regulator of P_{BAD}. In the presence of arabinose, the AraC protein is a positive regulatory element that allows expression from P_{BAD}. In the absence of arabinose, the AraC protein represses expression from P_{BAD}). This can lead to a 1,200-fold difference in the level of expression from P_{BAD}.

Other enteric bacteria contain arabinose regulatory systems homologous to the araC-araBAD system from E. coli. For example, there is homology at the amino acid sequence level between the E. coli and the S. Typhimurium AraC proteins, and less homology at the DNA level. However, there is high specificity in the activity of the AraC proteins. For example, the E. coli AraC protein activates only E. coli P_{BAD} (in the presence of arabinose) and not S. Typhimurium P_{BAD}. Thus, an arabinose regulated promoter may be used in a recombinant bacterium that possesses a similar arabinose operon, without substantial interference between the two, if the promoter and the operon are derived from two different species of bacteria.

Generally speaking, the concentration of arabinose necessary to induce expression is typically less than about 2%. In some embodiments, the concentration is less than about 1.5%, 1%, 0.5%, 0.2%, 0.1%, or 0.05%. In other embodiments, the concentration is 0.05% or below, e.g. about 0.04%, 0.03%, 0.02%, or 0.01%. In an exemplary embodiment, the concentration is about 0.05%.

In other embodiments, the promoter may be responsive to the level of maltose in the environment. Generally speaking, maltose may be present during the in vitro growth of a bacterium, while typically absent from host tissue. The malT nucleic acid sequence encodes MalT, a positive regulator of four maltose-responsive promoters (P_{PQ}, P_{EFG}, P_{KBM}, and P_{S}). The combination of malT and a mal promoter creates a tightly regulated expression system that has been shown to work as a strong promoter induced by the addition of maltose (6). Unlike the araC-P_{BAD} system, malT is expressed from a promoter (P_{T}) functionally unconnected to the other mal promoters. P_{T} is not regulated by MalT. The malEFG-malKBM promoter is a bidirectional promoter controlling expression of the malKBM nucleic acid sequences in one direction, and the malEFG nucleic acid sequences in the other direction. For convenience, the portion of the malEFG-malKBM promoter that mediates expression of the malKBM nucleic acid sequence, and which is controlled by the malT nucleic acid sequence product, is referred to herein as P_{KBM}, and the portion of the malEFG-malKBM promoter that mediates expression of the malEFG nucleic acid sequence, and that is controlled by the malT nucleic acid sequence product, is referred to herein as P_{EFG}. Full induction of P_{KBM} requires the presence of the MalT binding sites of P_{EFG}. For use in the vectors and systems described herein, a cassette with the malT nucleic acid sequence and one of the mal promoters may be used. This cassette is referred to herein as malT-Pₘₐₗ. In the presence of maltose, the MalT protein is a positive regulatory element that allows expression from Pₘₐₗ.

In still other embodiments, the promoter may be sensitive to the level of rhamnose in the environment. Analogous to the araC-P_{BAD} system described above, the rhaRS-P_{rhaB} activator-promoter system is tightly regulated by rhamnose. Expression from the rhamnose promoter (Pᵣₕₐ) is induced to high levels by the addition of rhamnose, which is common in bacteria but rarely found in host tissues. The nucleic acid sequences rhaBAD are organized in one operon that is controlled by the P_{rhaBAD} promoter. This promoter is regulated by two activators, RhaS and RhaR, and the corresponding nucleic acid sequences belong to one transcription unit that is located in the opposite direction of the rhaBAD nucleic acid sequences. If L-rhamnose is available, RhaR binds to the P_{rhaRS} promoter and activates the production of RhaR and RhaS. RhaS together with L-rhamnose in turn binds to the P_{rhaBAD} and the P_{rhaT} promoter and activates the transcription of the structural nucleic acid sequences. Full induction of rhaBAD transcription also requires binding of the Crp-cAMP complex, which is a key regulator of catabolite repression.

Although both L-arabinose and L-rhamnose act directly as inducers for expression of regulons for their catabolism, important differences exist in regard to the regulatory mechanisms. L-Arabinose acts as an inducer with the activator AraC in the positive control of the arabinose regulon. However, the L-rhamnose regulon is subject to a regulatory cascade; it is therefore subject to even tighter control than the araC P_{BAD} system. L-Rhamnose acts as an inducer with the activator RhaR for synthesis of RhaS, which in turn acts as an activator in the positive control of the rhamnose regulon. In the present invention, rhamnose may be used to interact with the RhaR protein and then the RhaS protein may activate transcription of a nucleic acid sequence operably-linked to the P_{rhaBAD} promoter.

In still other embodiments, the promoter may be sensitive to the level of xylose in the environment. The xylR-P_{xylA} system is another well-established inducible activator-promoter system. Xylose induces xylose-specific operons (xylE, xylFGHR, and xylAB) regulated by XylR and the cyclic AMP-Crp system. The XylR protein serves as a positive regulator by binding to two distinct regions of the xyl nucleic acid sequence promoters. As with the araC-P_{BAD} system described above, the xylR-P_{xylAB} and/or xylR-P_{xylFGH} regulatory systems may be used in the present invention. In these embodiments, xylR P_{xylAB} xylose interacting with the XylR protein activates transcription of nucleic acid sequences operably-linked to either of the two P_{xyl} promoters.

The nucleic acid sequences of the promoters detailed herein are known in the art, and methods of operably-linking them to a chromosomally integrated nucleic acid sequence encoding a repressor are known in the art and detailed in the examples.

### 3. Modification to Optimize Expression

A nucleic acid sequence encoding a repressor and regulatable promoter detailed above, for use in the present invention, may be modified so as to optimize the expression level of the nucleic acid sequence encoding the repressor. The optimal level of expression of the nucleic acid sequence encoding the repressor may be estimated, or may be determined by experimentation (see the Examples). Such a determination should take into consideration whether the repressor acts as a monomer, dimer, trimer, tetramer, or higher multiple, and should also take into consideration the copy number of the vector encoding the antigen of interest, as detailed below. In an exemplary embodiment, the level of expression is optimized so that the repressor is synthesized while in the permissive environment (i.e. in vitro growth) at a level that substantially inhibits the expression of the nucleic acid sequence encoding an antigen of interest, and is substantially not synthesized in a non-permissive environment, thereby allowing expression of the nucleic acid sequence encoding an antigen of interest.

As stated above, the level of expression may be optimized by modifying the nucleic acid sequence encoding the repressor and/or promoter. As used herein, “modify” refers to an alteration of the nucleic acid sequence of the repressor and/or promoter that results in a change in the level of transcription of the nucleic acid sequence encoding the repressor, or that results in a change in the level of synthesis of the repressor. For instance, in one embodiment, modify may refer to altering the start codon of the nucleic acid sequence encoding the repressor. Generally speaking, a GTG or TTG start codon, as opposed to an ATG start codon, may decrease translation efficiency ten-fold. In another embodiment, modify may refer to altering the Shine-Dalgarno (SD) sequence of the nucleic acid sequence encoding the repressor. The SD sequence is a ribosomal binding site generally located 6-7 nucleotides upstream of the start codon. The SD consensus sequence is AGGAGG, and variations of the consensus sequence may alter translation efficiency. In yet another embodiment, modify may refer to altering the distance between the SD sequence and the start codon. In still another embodiment, modify may refer to altering the −35 sequence for RNA polymerase recognition. In a similar embodiment, modify may refer to altering the −10 sequence for RNA polymerase binding. In an additional embodiment, modify may refer to altering the number of nucleotides between the −35 and −10 sequences. In an alternative embodiment, modify may refer to optimizing the codons of the nucleic acid sequence encoding the repressor to alter the level of translation of the mRNA encoding the repressor. For instance, non-A rich codons initially after the start codon of the nucleic acid sequence encoding the repressor may not maximize translation of the mRNA encoding the repressor. Similarly, the codons of the nucleic acid sequence encoding the repressor may be altered so as to mimic the codons from highly synthesized proteins of a particular organism. In a further embodiment, modify may refer to altering the GC content of the nucleic acid sequence encoding the repressor to change the level of translation of the mRNA encoding the repressor.

In some embodiments, more than one modification or type of modification may be performed to optimize the expression level of the nucleic acid sequence encoding the repressor. For instance, at least one, two, three, four, five, six, seven, eight, or nine modifications, or types of modifications, may be performed to optimize the expression level of the nucleic acid sequence encoding the repressor.

By way of non-limiting example, when the repressor is LacI, then the nucleic acid sequence of LacI and the promoter may be altered so as to increase the level of LacI synthesis. In one embodiment, the start codon of the LacI repressor may be altered from GTG to ATG. In another embodiment, the SD sequence may be altered from AGGG to AGGA. In yet another embodiment, the codons of lacI may be optimized according to the codon usage for highly synthesized proteins of Salmonella. In a further embodiment, the start codon of lacI may be altered, the SD sequence may be altered, and the codons of lacI may be optimized.

Methods of modifying the nucleic acid sequence encoding the repressor and/or the regulatable promoter are known in the art and detailed in the examples.

### 4. Transcription Termination Sequence

In some embodiments, the chromosomally integrated nucleic acid sequence encoding the repressor further comprises a transcription termination sequence. A transcription termination sequence may be included to prevent inappropriate expression of nucleic acid sequences adjacent to the chromosomally integrated nucleic acid sequence encoding the repressor and regulatable promoter.

### B. Vector

A recombinant bacterium of the invention that is capable of the regulated expression of at least one nucleic acid sequence encoding an antigen comprises, in part, a vector. The vector comprises a nucleic acid sequence encoding at least one antigen of interest operably linked to a promoter. The promoter is regulated by the chromosomally encoded repressor, such that the expression of the nucleic acid sequence encoding an antigen is repressed during in vitro growth of the bacterium, but the bacterium is capable of high level synthesis of the antigen in an animal or human host. In certain embodiments, however, the promoter may also be regulated by a plasmid-encoded repressor.

As used herein, “vector” refers to an autonomously replicating nucleic acid unit. The present invention can be practiced with any known type of vector, including viral, cosmid, phasmid, and plasmid vectors. The most preferred type of vector is a plasmid vector.

As is well known in the art, plasmids and other vectors may possess a wide array of promoters, multiple cloning sequences, transcription terminators, etc., and vectors may be selected so as to control the level of expression of the nucleic acid sequence encoding an antigen by controlling the relative copy number of the vector. In some instances in which the vector might encode a surface localized adhesin as the antigen, or an antigen capable of stimulating T-cell immunity, it may be preferable to use a vector with a low copy number such as at least two, three, four, five, six, seven, eight, nine, or ten copies per bacterial cell. A non-limiting example of a low copy number vector may be a vector comprising the pSC101 ori.

In other cases, an intermediate copy number vector might be optimal for inducing desired immune responses. For instance, an intermediate copy number vector may have at least 10, 11, 12, 13, 14, 15, 16, 17, 18, 19, 20, 21, 22, 23, 24, 25, 26, 27, 28, 29, or 30 copies per bacterial cell. A non-limiting example of an intermediate copy number vector may be a vector comprising the p15A ori.

In still other cases, a high copy number vector might be optimal for the induction of maximal antibody responses. A high copy number vector may have at least 31, 35, 40, 45, 50, 55, 60, 65, 70, 75, 80, 85, 90, 95, or 100 copies per bacterial cell. In some embodiments, a high copy number vector may have at least 100, 125, 150, 175, 200, 225, 250, 275, 300, 325, 350, 375, or 400 copies per bacterial cell. Non-limiting examples of high copy number vectors may include a vector comprising the pBR on or the pUC ori.

Additionally, vector copy number may be increased by selecting for mutations that increase plasmid copy number. These mutations may occur in the bacterial chromosome but are more likely to occur in the plasmid vector.

Preferably, vectors used herein do not comprise antibiotic resistance markers to select for maintenance of the vector.

### 1. Antigen

As used herein, “antigen” refers to a biomolecule capable of eliciting an immune response in a host. In some embodiments, an antigen may be a protein, or fragment of a protein, or a nucleic acid. In an exemplary embodiment, the antigen elicits a protective immune response. As used herein, “protective” means that the immune response contributes to the lessening of any symptoms associated with infection of a host with the pathogen the antigen was derived from or designed to elicit a response against or reduces the persistence of the pathogen in the host. For example, a protective antigen from a pathogen, such as Mycobacterium, may induce an immune response that helps to ameliorate symptoms associated with Mycobacterium infection or reduce the morbidity and mortality associated with infection with the pathogen. The use of the term “protective” in this invention does not necessarily require that the host is completely protected from the effects of the pathogen.

Antigens may be from bacterial, viral, mycotic and parasitic pathogens, and may be designed to protect against bacterial, viral, mycotic, and parasitic infections, respectively. Alternatively, antigens may be derived from gametes, provided they are gamete specific, and may be designed to block fertilization. In another alternative, antigens may be tumor antigens, and may be designed to decrease tumor growth. It is specifically contemplated that antigens from organisms newly identified or newly associated with a disease or pathogenic condition, or new or emerging pathogens of animals or humans, including those now known or identified in the future, may be expressed by a bacterium detailed herein. Furthermore, antigens for use in the invention are not limited to those from pathogenic organisms. Immunogenicity of the bacterium may be augmented and/or modulated by constructing strains that also express sequences for cytokines, adjuvants, and other immunomodulators.

Some examples of microorganisms useful as a source for antigen are listed below. These may include microoganisms for the control of plague caused by Yersinia pestis and other Yersinia species such as Y. pseudotuberculosis and Y. enterocolitica, for the control of gonorrhea caused by Neisseria gonorrhoea, for the control of syphilis caused by Treponema pallidum, and for the control of venereal diseases as well as eye infections caused by Chlamydia trachomatis. Species of Streptococcus from both group A and group B, such as those species that cause sore throat or heart diseases, Erysipelothrix rhusiopathiae, Neisseria meningitidis, Mycoplasma pneumoniae and other Mycoplasma-species, Hemophilus influenza, Bordetella pertussis, Mycobacterium tuberculosis, Mycobacterium leprae, other Bordetella species, Escherichia coli, Streptococcus equi, Streptococcus pneumoniae, Brucella abortus, Pasteurella hemolytica and P. multocida, Vibrio cholera, Shigella species, Borrellia species, Bartonella species, Heliobacter pylori, Campylobacter species, Pseudomonas species, Moraxella species, Brucella species, Francisella species, Aeromonas species, Actinobacillus species, Clostridium species, Rickettsia species, Bacillus species, Coxiella species, Ehrlichia species, Listeria species, and Legionella pneumophila are additional examples of bacteria within the scope of this invention from which antigen nucleic acid sequences could be obtained. Viral antigens may also be used. Viral antigens may be used in antigen delivery microorganisms directed against viruses, either DNA or RNA viruses, for example from the classes Papovavirus, Adenovirus, Herpesvirus, Poxvirus, Parvovirus, Reovirus, Picornavirus, Myxovirus, Paramyxovirus, Flavivirus or Retrovirus. In one embodiment, the antigen is an influenza antigen. Antigens may also be derived from pathogenic fungi, protozoa and parasites. For instance, by way of non-limiting example, the antigen may be an Eimeria antigen, a Plasmodium antigen, or a Taenia solium antigen.

Certain embodiments encompass an allergen as an antigen. Allergens are substances that cause allergic reactions in a host that is exposed to them. Allergic reactions, also known as Type I hypersensitivity or immediate hypersensitivity, are vertebrate immune responses characterized by IgE production in conjunction with certain cellular immune reactions. Many different materials may be allergens, such as animal dander and pollen, and the allergic reaction of individual hosts will vary for any particular allergen. It is possible to induce tolerance to an allergen in a host that normally shows an allergic response. The methods of inducing tolerance are well-known and generally comprise administering the allergen to the host in increasing dosages.

It is not necessary that the vector comprise the complete nucleic acid sequence of the antigen. It is only necessary that the antigen sequence used be capable of eliciting an immune response. The antigen may be one that was not found in that exact form in the parent organism. For example, a sequence coding for an antigen comprising 100 amino acid residues may be transferred in part into a recombinant bacterium so that a peptide comprising only 75, 65, 55, 45, 35, 25, 15, or even 10, amino acid residues is produced by the recombinant bacterium. Alternatively, if the amino acid sequence of a particular antigen or fragment thereof is known, it may be possible to chemically synthesize the nucleic acid fragment or analog thereof by means of automated nucleic acid sequence synthesizers, PCR, or the like and introduce said nucleic acid sequence into the appropriate copy number vector.

In another alternative, a vector may comprise a long sequence of nucleic acid encoding several nucleic acid sequence products, one or all of which may be antigenic. In some embodiments, a vector of the invention may comprise a nucleic acid sequence encoding at least one antigen, at least two antigens, at least three antigens, or more than three antigens. These antigens may be encoded by two or more open reading frames operably linked to be expressed coordinately as an operon, wherein each antigen is synthesized independently. Alternatively, the two or more antigens may be encoded by a single open reading frame such that the antigens are synthesized as a fusion protein.

In certain embodiments, an antigen of the invention may comprise a B cell epitope or a T cell epitope. Alternatively, an antigen to which an immune response is desired may be expressed as a fusion to a carrier protein that contains a strong promiscuous T cell epitope and/or serves as an adjuvant and/or facilitates presentation of the antigen to enhance, in all cases, the immune response to the antigen or its component part. This can be accomplished by methods known in the art. Fusion to tenus toxin fragment C, CT-B, LT-B and hepatitis virus B or woodchuck hepatitis core are particularly useful for these purposes, although other epitope presentation systems are well known in the art.

In further embodiments, a nucleic acid sequence encoding an antigen of the invention may comprise a secretion signal. In other embodiments, an antigen of the invention may be toxic to the recombinant bacterium.

### 2. Promoter Regulated by Repressor

The vector comprises a nucleic acid sequence encoding at least one antigen operably-linked to a promoter regulated by the repressor, encoded by a chromosomally integrated nucleic acid sequence. One of skill in the art would recognize, therefore, that the selection of a repressor dictates, in part, the selection of the promoter operably-linked to a nucleic acid sequence encoding an antigen of interest. For instance, if the repressor is LacI, then the promoter may be selected from the group consisting of LacI responsive promoters, such as P_{trc}, P_{lac}, P_{T7lac} and P_{tac}. If the repressor is C2, then the promoter may be selected from the group consisting of C2 responsive promoters, such as P22 promoters P_{L} and P_{R}. If the repressor is C1, then the promoter may be selected from the group consisting of C1 responsive promoters, such as λ promoters P_{L} and P_{R}.

In each embodiment herein, the promoter regulates expression of a nucleic acid sequence encoding the antigen, such that expression of the nucleic acid sequence encoding an antigen is repressed when the repressor is synthesized (i.e. during in vitro growth of the bacterium), but expression of the nucleic acid sequence encoding an antigen is high when the repressor is not synthesized (i.e. in an animal or human host). Generally speaking, the concentration of the repressor will decrease with every cell division after expression of the nucleic acid sequence encoding the repressor ceases. In some embodiments, the concentration of the repressor decreases enough to allow high level expression of the nucleic acid sequence encoding an antigen after about 2, 3, 4, 5, 6, 7, 8, 9, 10, 11, or 12 divisions of the bacterium. In an exemplary embodiment, the concentration of the repressor decreases enough to allow high level expression of the nucleic acid sequence encoding an antigen after about 5 divisions of the bacterium in an animal or human host.

In certain embodiments, the promoter may comprise other regulatory elements. For instance, the promoter may comprise lacO if the repressor is LacI. This is the case with the lipoprotein promoter Pₗₚₚ that is regulated by LacI since it possesses the LacI binding domain lacO.

In one embodiment, the repressor is a LacI repressor and the promoter is P_{trc}.

### 3. Expression of the Nucleic Acid Sequence Encoding an Antigen

As detailed above, generally speaking the expression of the nucleic acid sequence encoding the antigen should be repressed when the repressor is synthesized. For instance, if the repressor is synthesized during in vitro growth of the bacterium, expression of the nucleic acid sequence encoding the antigen should be repressed. Expression may be “repressed” or “partially repressed” when it is about 50%, 45%, 40%, 35%, 30%, 25%, 20%, 15%, 10%, 5%, 1%, or even less than 1% of the expression under non-repressed conditions. Thus although the level of expression under conditions of “complete repression” might be exceeding low, it is likely to be detectable using very sensitive methods since repression can never by absolute.

Conversely, the expression of the nucleic acid sequence encoding the antigen should be high when the expression of the nucleic acid sequence encoding the repressor is repressed. For instance, if the nucleic acid sequence encoding the repressor is not expressed during growth of the recombinant bacterium in the host, the expression of the nucleic acid sequence encoding the antigen should be high. As used herein, “high level” expression refers to expression that is strong enough to elicit an immune response to the antigen. Consequently, the copy number correlating with high level expression can and will vary depending on the antigen and the type of immune response desired. Methods of determining whether an antigen elicits an immune response such as by measuring antibody levels or antigen-dependant T cell populations or antigen-dependant cytokine levels are known in the art, and methods of measuring levels of expression of antigen encoding sequences by measuring levels of mRNA transcribed or by quantitating the level of antigen synthesis are also known in the art.

ii. Eight Unit Viral Vector

A single expression vector capable of generating an attenuated virus from a segmented genome has been developed. An auxotrophic bacterial carrier can carry and deliver this expression vector into in vitro cultured cells, resulting in the recovery of virus, either attenuated or non-attenuated. Advantageously, the expression vector is stable in bacteria at 37° C., and produces higher titers of virus than traditional multi-vector systems when transfected into eukaryotic cells.

The expression vector generally comprises a plasmid having at least two types of transcription cassettes. One type of transcription cassette is designed for vRNA production. The other type of transcription cassette is designed for the production of both vRNAs, and mRNAs. As will be appreciated by a skilled artisan, the number of transcription cassettes, and their placement within the vector relative to each other, can and will vary depending on the segmented virus that is produced. Each of these components of the expression vector is described in more detail below.

The expression vector may be utilized to produce several different segmented and nonsegmented viruses. Viruses that may be produced from the expression vector include positive-sense RNA viruses, negative-sense RNA viruses and double-stranded RNA (ds-RNA) viruses.

In one embodiment, the virus may be a positive-sense RNA virus. Non-limiting examples of positive-sense RNA virus may include viruses of the family Arteriviridae, Caliciviridae, Coronaviridae, Flaviviridae, Picornaviridae, Roniviridae, and Togaviridae. Non-limiting examples of positive-sense RNA viruses may include SARS-coronavirus, Dengue fever virus, hepatitis A virus, hepatitis C virus, Norwalk virus, rubella virus, West Nile virus, Sindbis virus, Semliki forest virus and yellow fever virus.

In one embodiment, the virus may be a double-stranded RNA virus. Non-limiting examples of segmented double-stranded RNA viruses may include viruses of the family Reoviridae and may include aquareovirus, blue tongue virus, coltivirus, cypovirus, fijivirus, idnoreovirus, mycoreovirus, orbivirus, orthoreovirus, oryzavirus, phytoreovirus, rotavirus and seadornavirus.

In yet another embodiment, the virus may be a negative-sense RNA virus. Negative-sense RNA viruses may be viruses belonging to the families Orthomyxoviridae, Bunyaviridae, and Arenaviridae with six-to-eight, three, or two negative-sense vRNA segments, respectively. Non-limiting examples of negative-sense RNA viruses may include thogotovirus, isavirus, bunyavirus, hantavirus, nairovirus, phlebovirus, tospovirus, tenuivirus, ophiovirus, arenavirus, deltavirus and influenza virus.

In another aspect, the invention provides an expression vector capable of generating influenza virus. There are three known genera of influenza virus: influenza A virus, influenza B virus and influenza C virus. Each of these types of influenza viruses may be produced utilizing the single expression vector of the invention.

In one exemplary embodiment, the expression vector is utilized to produce Influenza A virus. Influenza A viruses possess a genome of 8 vRNA segments, including PA, PB1, PB2, HA, NP, NA, M and NS, which encode a total of ten to eleven proteins. To initiate the replication cycle, vRNAs and viral replication proteins must form viral ribonucleoproteins (RNPs). The influenza RNPs consist of the negative-sense viral RNAs (vRNAs) encapsidated by the viral nucleoprotein, and the viral polymerase complex, which is formed by the PA, PB1 and PB2 proteins. The RNA polymerase complex catalyzes three different reactions: synthesis of an mRNA with a 5′ cap and 3′ polyA structure essential for translation by the host translation machinery; a full length complementary RNA (cRNA), and of genomic vRNAs using the cRNAs as a template. Newly synthesized vRNAs, NP and, PB1, PB2 and PA polymerase proteins are then assembled into new RNPs, for further replication or encapsidation and release of progeny virus particles. Therefore, to produce influenza virus using a reverse genetics system, all 8 vRNAs and mRNAs that express the viral proteins (NP, PB1, PB1 and PA) essential for replication must be synthesized. The expression vector of the invention may be utilized to produce all of these vRNAs and mRNAs.

The expression vector may also be utilized to produce any serotype of influenza A virus without departing from the scope of the invention. Influenza A viruses are classified into serotypes based upon the antibody response to the viral surface proteins hemagglutinin (HA or H) encoded by the HA vRNA segment, and neuraminidase (NA or N) encoded by the NA vRNA segment. At least sixteen H subtypes (or serotypes) and nine N subtypes of influenza A virus have been identified. New influenza viruses are constantly being produced by mutation or by reassortment of the 8 vRNA segments when more than one influenza virus infects a single host. By way of example, known influenza serotypes may include H1N1, H1N2, H2N2, H3N1, H3N2, H3N8, H5N1, H5N2, H5N3, H5N8, H5N9, H7N1, H7N2, H7N3, H7N4, H7N7, H9N2, and H10N7 serotypes.

### A. Vector

The expression vector of the invention comprises a vector. As used in reference to the eight unit viral vector, “vector” refers to an autonomously replicating nucleic acid unit. The present invention can be practiced with any known type of vector, including viral, cosmid, phasmid, and plasmid vectors. The most preferred type of vector is a plasmid vector. As is well known in the art, plasmids and other vectors may possess a wide array of promoters, multiple cloning sequences, and transcription terminators.

The vector may have a high copy number, an intermediate copy number, or a low copy number. The copy number may be utilized to control the expression level for the transcription cassettes, and as a means to control the expression vector's stability. In one embodiment, a high copy number vector may be utilized. A high copy number vector may have at least 31, 35, 40, 45, 50, 55, 60, 65, 70, 75, 80, 85, 90, 95, or 100 copies per bacterial cell. In other embodiments, the high copy number vector may have at least 100, 125, 150, 175, 200, 225, 250, 275, 300, 325, 350, 375, or 400 copies per bacterial cell. Non-limiting examples of high copy number vectors may include a vector comprising the pBR on or the pUC ori. In an alternative embodiment, a low copy number vector may be utilized. For example, a low copy number vector may have one or at least two, three, four, five, six, seven, eight, nine, or ten copies per bacterial cell. A non-limiting example of low copy number vector may be a vector comprising the pSC101 ori. In an exemplary embodiment, an intermediate copy number vector may be used. For instance, an intermediate copy number vector may have at least 10, 11, 12, 13, 14, 15, 16, 17, 18, 19, 20, 21, 22, 23, 24, 25, 26, 27, 28, 29, or 30 copies per bacterial cell. A non-limiting example of an intermediate copy number vector may be a vector comprising the p15A ori.

The vector may further comprise a selectable marker. Generally speaking, a selectable marker encodes a product that the host cell cannot make, such that the cell acquires resistance to a specific compound or is able to survive under specific conditions. For example, the marker may code for an antibiotic resistance factor. Suitable examples of antibiotic resistance markers include, but are not limited to, those coding for proteins that impart resistance to kanamycin, spectomycin, neomycin, geneticin (G418), ampicillin, tetracycline, and chlorampenicol. The selectable marker may code for proteins that confer resistance to herbicides, such as chlorsulfuron or phosphinotricin acetyltransferase. Other appropriate selectable markers include the thymidine kinase (tk) and the adenine phosphoribosyltransferase (apr) genes, which enable selection in tk- and apr-cells, respectively, and the dihydrofloate reductase (dhfr) genes that confer resistance to methotrexate or trimethoprim. In still other cases, the vector might have selectable Asd⁺, MurA⁺, AroA⁺, DadB⁺, Alr⁺, AroC⁺, AroD⁺, IIvC⁺ and/or IIvE⁺ when the expression vector is used in a balanced-lethal or balanced-attenuation vector-host system when present in and delivered by carrier bacteria.

In some embodiments, the vector may also comprise a transcription cassette for expressing non-viral reporter proteins. By way of example, reporter proteins may include a fluorescent protein, luciferase, alkaline phosphatase, beta-galactosidase, beta-lactamase, horseradish peroxidase, and variants thereof.

In some embodiments, the vector may also comprise a DNA nuclear targeting sequence (DTS). A non-limiting example of a DTS may include the SV40 DNA nuclear targeting sequence. In other embodiments, the vector may also comprise an artificial binding site for a transcriptional factor, such as NF-κB and/or AP-2 (SEQ ID NO: xx: GGGGACTTTCCGGGGACTTTCCTCCC CACGCGGGGGACTTTCCGCCACGGGCGGGGACTTTCCGGGGACTTTCC). Transcription factor NF-κB is found in almost all animal cell types. Salmonella infection stimulates the expression NF-κB rapidly, and binding affinity of NF-κB members to their DNA-binding sites (κB sites) is high and the translocation of NF-κB-DNA complex into the nucleus is rapid (minutes). The plasmid DNA with κB sites allows newly synthesized NF-κB to bind to the plasmid DNA in the cytoplasm and transport it to the nucleus through the protein nuclear import machinery. Depending on their position relative to the trans-gene, the binding sites could also act as transcriptional enhancers that further increase gene expression levels. The SV40 DTS, NF-κB, and AP-2 binding sequence facilitate nuclear import of the plasmid DNA, and this facilitates transcription of genetic sequences on the vector.

B. Transcription Cassettes for vRNAs Expression

The expression vector comprises at least one transcription cassette for vRNA production. Generally speaking, the transcription cassette for vRNA production minimally comprises a Poll promoter operably linked to a viral cDNA linked to a Poll transcription termination sequence. In an exemplary embodiment, the transcription cassette may also include a nuclear targeting sequence. The number of transcription cassettes for vRNA production within the expression vector can and will vary depending on the virus that is produced. For example, the expression vector may comprise two, three, four, five, six, seven, or eight or more transcription cassettes for vRNA production. When the virus that is produced is influenza, the vector typically will comprise four transcription cassettes for vRNA production.

The term “viral cDNA”, as used herein, refers to a copy of deoxyribonucleic acid (cDNA) sequence corresponding to a vRNA segment of an RNA virus genome. cDNA copies of viral RNA segments may be derived from vRNAs using standard molecular biology techniques known in the art (see, e.g., Sambrook et al. (1989) “Molecular Cloning: A Laboratory Manual,” 2nd Ed., Cold Spring Harbor Laboratory Press, Cold Spring Harbor, and Knipe et al (2006) “Virology”, Fifth Edition, Lippincott Williams & Wilkins; edition). In some embodiments, the cDNA may be derived from a naturally occurring virus strain or a virus strain commonly used in vitro. In other embodiments, the cDNA may be derived synthetically by generating the cDNA sequence in vitro using methods known in the art. The natural or synthetic cDNA sequence may further be altered to introduce mutations and sequence changes. By way of example, a naturally occurring viral sequence may be altered to attenuate a virus, to adapt a virus for in vitro culture, or to tag the encoded viral proteins.

The selection of promoter can and will vary. The term “promoter”, as used in reference to a viral cassette, may mean a synthetic or naturally derived molecule that is capable of conferring, activating or enhancing expression of a nucleic acid. A promoter may comprise one or more specific transcriptional regulatory sequences to further enhance expression and/or to alter the spatial expression and/or temporal expression of a nucleic acid. The promoters may be of viral, prokaryotic, phage or eukaryotic origin. Non-limiting examples of promoters may include T7 promoter, T3 promoter, SP6 promoter, RNA polymerase I promoter and combinations thereof. In some embodiments, the promoters may be different in each transcription cassette. In preferred embodiments, the promoters may be the same in each transcription cassette. In preferred alternatives of this embodiment, the promoters may be RNA polymerase I (Pol I) promoters. In an exemplary alternative of this embodiment, the promoters may be human Pol I promoters. In another exemplary alternative of this embodiment, the promoters may be chicken Pol I promoters.

The promoter may be operably linked to the cDNA to produce a negative-sense vRNA or a positive-sense cRNA. In an exemplary alternative of this embodiment, the promoter may be operably linked to the cDNA to produce a negative-sense vRNA.

The transcription cassette also includes a terminator sequence, which causes transcriptional termination at the end of the viral cDNA sequence. By way of a non-limiting example, terminator sequences suitable for the invention may include a Pol I terminator, the late SV40 polyadenylation signal, the CMV polyadenylation signal, the bovine growth hormone polyadenylation signal, or a synthetic polyadenylation signal. In some embodiments, the terminators may be different in each transcription cassette. In a preferred embodiment, the terminators may be the same in each transcription cassette. In one alternative of this embodiment, the Pol I terminator may be a human Pol I terminator. In an exemplary embodiment, the terminator is a murine Pol I terminator. In an exemplary alternative of this embodiment, the terminator sequence of the expression cassettes may be a truncated version of the murine Pol I terminator.

To function properly during replication, vRNAs transcribed from the transcription cassettes generally have precise 5′ and 3′ ends that do not comprise an excess of non-virus sequences. Depending on the promoters and terminators used, this may be accomplished by precise fusion to promoters and terminators or, by way of example, the transcription cassette may comprise ribozymes at the ends of transcripts, wherein the ribozymes cleave the transcript in such a way that the sequences of the 5′ and 3′ termini are generated as found in the vRNA.

As will be appreciated by a skilled artisan, when the expression vector produces influenza virus, the expression vector may comprise at least one transcription cassette for vRNA production. The transcription cassette may be selected from the group consisting of (1) a Pol I promoter operably linked to an influenza virus HA cDNA linked to a Pol I transcription termination sequence; (2) a Pol I promoter operably linked to an influenza virus NA cDNA linked to a Pol I transcription termination sequence; (3) a Pol I promoter operably linked to an influenza virus M cDNA linked to a Pol I transcription termination sequence; and (4) a Poll promoter operably linked to an influenza virus NS cDNA linked to a Pol I transcription termination sequence. The expression vector may comprise at least 2, 3, or 4 of these transcription cassettes. In an exemplary embodiment, the expression vector will also include either one or two different nuclear targeting sequences (e.g., SV40 DTS and an artificial binding sequence for a transcriptional factor such as NF-κB and/or AP-2).

In an exemplary embodiment when the expression vector produces influenza virus, the expression vector will comprise four transcription cassettes for vRNA production. The transcription cassettes for this embodiment will comprise (1) a Pol I promoter operably linked to an influenza virus HA cDNA linked to a Pol I transcription termination sequence; (2) a Pol I promoter operably linked to an influenza virus NA cDNA linked to a Pol I transcription termination sequence; (3) a Pol I promoter operably linked to an influenza virus M cDNA linked to a Poll transcription termination sequence; and (4) a Poll promoter operably linked to an influenza virus NS cDNA linked to a Poll transcription termination sequence. In an exemplary embodiment, the expression vector will also include either one or two different nuclear targeting sequences (e.g., SV40 DTS and an artificial binding sequence for a transcriptional factor such as NF-κB and/or AP-2).

C. Transcription Cassettes for vRNA and mRNA Expression

The expression vector comprises at least one transcription cassette for vRNA and mRNA production. Typically, the transcription cassette for vRNA and mRNA production minimally comprises a Pol I promoter operably linked to a viral cDNA linked to a Pol I transcription termination sequence, and a PoIII promoter operably linked to the viral cDNA and a PoIII transcription termination sequence. In an exemplary embodiment, the transcription cassette will also include a nuclear targeting sequence. The number of transcription cassettes for vRNA and mRNA production within the expression vector can and will vary depending on the virus that is produced. For example, the expression vector may comprise two, three, four, five, six, seven, or eight or more transcription cassettes for vRNA and mRNA production. When the virus that is produced is influenza, the expression cassette typically may comprise four transcription cassettes for vRNA and mRNA production.

The viral cDNA, Pol I promoter and Pol I terminator suitable for producing vRNA is as described above in section (e)iiB.

For mRNA production, each transcription cassette comprises a Pol II promoter operably linked to cDNA and a Pol II termination sequence. Non-limiting examples of promoters may include the cytomegalovirus (CMV) promoter, Rous sarcoma virus (RSV) promoter, simian virus 40 (SV40) early promoter, ubiquitin C promoter or the elongation factor 1 alpha (EF1α) promoter. In some embodiments, the promoters may be different in each transcription cassette. In preferred embodiments, the promoters may be the same in each transcription cassette. In preferred alternatives of this embodiment, the promoters may be the CMV Pol II promoter.

Each transcription cassette also comprises a Pol II terminator sequence. By way of non-limiting example, terminator sequences suitable for the invention may include the late SV40 polyadenylation signal, the CMV polyadenylation signal, the bovine growth hormone (BGH) polyadenylation signal, or a synthetic polyadenylation signal. In some embodiments, the terminators may be different in each transcription cassette. In a preferred embodiment, the terminators may be the same in each transcription cassette. In an exemplary embodiment, the terminator is a BGH polyadenylation signal. In an exemplary alternative of this embodiment, the terminator sequence of the expression cassettes may be a truncated version of the BGH polyadenylation signal.

To function properly in initiating vRNA replication, mRNAs transcribed from the transcription cassettes may contain signals for proper translation by the host cell translation machinery. Most cellular mRNAs transcribed from a PoIII promoter are capped at the 5′ end and polyadenylated at the 3′ end after transcription to facilitate mRNA translation. However, some cellular mRNAs and many viral mRNAs encode other sequences that facilitate translation of the mRNA in the absence of a 5′ cap structure or 3′ polyA structure. By way of example, some cellular mRNAs and viral mRNAs may encode an internal ribosomal entry site (IRES), which could functionally replace the 5′ cap. By way of another example, some mRNAs and viral mRNAs may encode an RNA structure, such as a pseudoknot, at the 3′ end of the mRNA, which could functionally replace the 3′ polyA. In an exemplary embodiment, the mRNAs transcribed from the transcription cassettes are capped at the 5′ end and polyadenylated at the 3′ end.

As will be appreciated by a skilled artisan, when the expression vector produces influenza virus, the expression vector may comprise at least one transcription cassette for vRNA and mRNA production. The transcription cassette may be selected from the group consisting of (1) a Poll promoter operably linked to an influenza virus PA cDNA linked to a Pol I transcription termination sequence and a Pol II promoter operably linked to the PA cDNA and a Pol II transcription termination sequence; (2) a Pol I promoter operably linked to an influenza virus PB1 cDNA linked to a Poll transcription termination sequence and a Pol II promoter operably linked to the PB1 cDNA and a Pol II transcription termination sequence; (3) a Pol I promoter operably linked to an influenza virus PB2 cDNA linked to a Pol I transcription termination sequence and a Pol II promoter operably linked to the PB2 cDNA and a Pol II transcription termination sequence; and (4) a Pol I promoter operably linked to an influenza virus NP cDNA linked to a Pol I transcription termination sequence and a Pol II promoter operably linked to the NP cDNA and a Pol II transcription termination sequence. The expression vector may comprise at least 2, 3, or 4 of these transcription cassettes. In an exemplary embodiment, the expression vector will also include either one or two different nuclear targeting sequences (e.g., SV40 DTS or an artificial binding sequence for a transcriptional factor such as NF-κB and/or AP-2).

In an exemplary embodiment when the expression vector produces influenza virus, the expression vector will comprise four transcription cassettes for vRNA and mRNA production. The transcription cassettes for this embodiment will comprise (1) a Pol I promoter operably linked to an influenza virus PA cDNA linked to a Pol I transcription termination sequence and a Pol II promoter operably linked to the PA cDNA and a Pol II transcription termination sequence; (2) a Pol I promoter operably linked to an influenza virus PB1 cDNA linked to a Pol I transcription termination sequence and a Pol II promoter operably linked to the PB1 cDNA and a Pol II transcription termination sequence; (3) a Pol I promoter operably linked to an influenza virus PB2 cDNA linked to a Pol I transcription termination sequence and a Pol II promoter operably linked to the PB2 cDNA and a Pol II transcription termination sequence; and (4) a Pol I promoter operably linked to an influenza virus NP cDNA linked to a Pol I transcription termination sequence and a PoIII promoter operably linked to the NP cDNA and a Pol II transcription termination sequence. In an exemplary embodiment, each expression plasmid construct will also include either one or two different nuclear translocation signals (e.g., SV40 DTS or an artificial binding sequence for a transcriptional factor such as NF-κB and/or AP-2).

### D. Exemplary Expression Vectors

In an exemplary iteration of the invention, a single expression vector will comprise all of the genomic segments necessary for the production of influenza virus in a host cell. As detailed above, for the production of influenza virus HA, NA, NS, and M vRNA must be produced and PA, PB1, PB2, and NP vRNA and mRNA must be produced. For this iteration, the expression vector will comprise four transcription cassettes for vRNA production and four transcription cassettes for vRNA and mRNA production. The four cassettes for vRNA production will comprise (1) a Pol I promoter operably linked to an influenza virus HA cDNA linked to a Pol I transcription termination sequence; (2) a Pol I promoter operably linked to an influenza virus NA cDNA linked to a Poll transcription termination sequence; (3) a Pol I promoter operably linked to an influenza virus M cDNA linked to a Pol I transcription termination sequence; and (4) a Pol I promoter operably linked to an influenza virus NS cDNA linked to a Poll transcription termination sequence. The four transcription cassettes for vRNA and mRNA production will comprise (1) a Pol I promoter operably linked to an influenza virus PA cDNA linked to a Pol I transcription termination sequence and a Pol II promoter operably linked to the PA cDNA and a Pol II transcription termination sequence; (2) a Pol I promoter operably linked to an influenza virus PB1 cDNA linked to a Pol I transcription termination sequence and a Pol II promoter operably linked to the PB1 cDNA and a Pol II transcription termination sequence; (3) a Pol I promoter operably linked to an influenza virus PB2 cDNA linked to a Pol I transcription termination sequence and a Pol II promoter operably linked to the PB2 cDNA and a PoIII transcription termination sequence; and (4) a Pol I promoter operably linked to an influenza virus NP cDNA linked to a Pol I transcription termination sequence and a Pol II promoter operably linked to the NP cDNA and a PoIII transcription termination sequence. The expression vector will preferably also include either one or two different nuclear translocation signals (e.g., SV40 DTS or an artificial binding sequence for a transcriptional factor such as NF-κB and/or AP-2). In an exemplary embodiment, the vector is a plasmid. The plasmid will generally be a low or intermediate copy number plasmid.

The arrangement and direction of transcription cassettes within the single expression vector relative to each other can and will vary without departing from the scope of the invention. It is believed, however, without being bound by any particular theory that arrangement of transcription cassettes in pairs of vRNA cassettes and vRNA and mRNA cassettes is preferable because it may reduce the degree of recombination and as a result, yield an expression vector with increased genetic stability.

It is also envisioned that in certain embodiments, influenza genomic segments may be produced from more than a single expression vector without departing from the scope of the invention. The genomic segments may be produced, for example, from 2, 3, or 4 or more different expression vectors. In an iteration of this embodiment, NS, and M vRNA, and PA, PB1, PB2, and NP vRNA and mRNA are produced from a single expression vector. For this iteration, the expression vector will comprise two transcription cassettes for vRNA production and four transcription cassettes for vRNA and mRNA production. The two transcription cassettes for vRNA production will comprise (1) a Poll promoter operably linked to an influenza virus M cDNA linked to a Pol I transcription termination sequence; and (2) a Pol I promoter operably linked to an influenza virus NS cDNA linked to a Pol I transcription termination sequence. The four transcription cassettes for vRNA and mRNA production will comprise (1) a Pol I promoter operably linked to an influenza virus PA cDNA linked to a Pol I transcription termination sequence and a Pol II promoter operably linked to the PA cDNA and a Pol II transcription termination sequence; (2) a Pol I promoter operably linked to an influenza virus PB1 cDNA linked to a Pol I transcription termination sequence and a Pol II promoter operably linked to the PB1 cDNA and a Pol II transcription termination sequence; (3) a Pol I promoter operably linked to an influenza virus PB2 cDNA linked to a Pol I transcription termination sequence and a Pol II promoter operably linked to the PB2 cDNA and a Pol II transcription termination sequence; and (4) a Pol I promoter operably linked to an influenza virus NP cDNA linked to a Poll transcription termination sequence and a Pol II promoter operably linked to the NP cDNA and a Pol II transcription termination sequence. The expression of HA vRNA and NA vRNA may be from a single expression vector that comprises two transcription cassettes comprising (1) a Poll promoter operably linked to an influenza virus HA cDNA linked to a Poll transcription termination sequence; and (2) a Poll promoter operably linked to an influenza virus NA cDNA linked to a Pol I transcription termination sequence. Alternatively, expression of HA vRNA and NA vRNA may be from two separate expression vectors.

In some embodiments, restriction digestion sites may be placed at convenient locations in the expression vector. By way of example, restriction enzyme sites placed at the extremities of the cDNAs may be used to facilitate replacement of cDNA segments to produce a desired reassortment or strain of the virus. By way of another example, restriction enzyme sites placed at the extremities of the transcription cassettes may be used to facilitate replacement of transcription cassettes to produce a desired reassortment or strain of the virus. Suitable, endonuclease restriction sites include sites that are recognized by restriction enzymes that cleave double-stranded nucleic acid. By way of non-limiting example, these sites may include AarI, AccI, AgeI, Apa, BamHI, BglI, BglII, BsiWI, BssHI, BstBI, ClaI, CviQI, DdeI, DpnI, DraI, EagI, EcoRI, EcoRV, FseI, FspI, HaeII, HaeIII, HhaI, HincII, HindIII, HpaI, HpaII, KpnI, KspI, MboI, MfeI, NaeI, NarI, NcoI, NdeI, NgoMIV, NheI, NotI, PacI, PhoI, PmlI, PstI, PvuI, PvuII, SacI, SacII, SalI, SbfI, SmaI, SpeI, SphI, SrfI, StuI, TaqI, TfiI, TliI, XbaI, XhoI, XmaI, XmnI, and ZraI. In an exemplary alternative of this embodiment, the restriction enzyme site may be AarI.

iii. Nucleic Acid Vaccine Vector

A recombinant bacterium of the invention may encompass a nucleic acid vaccine vector. Such a vector is typically designed to be transcribed in the nucleus of the host cell to produce mRNA encoding one or more antigens of interest. To increase performance, a nucleic acid vaccine vector should be targeted to the nucleus of a host cell, and should be resistant to nuclease attack.

In one embodiment of the invention, a nucleic acid vaccine vector may be targeted to the nucleus using a DNA nuclear targeting sequence. Such a sequence allows transcription factors of the host cell to bind to the vector in the cytoplasm and escort it to the nucleus via the nuclear localization signal-mediated machinery. DNA nuclear targeting sequences are known in the art. For instance, the SV40 enhancer may be used. In particular, a single copy of a 72-bp element of the SV40 enhancer may be used, or a variation thereof. The SV40 enhancer may be used in combination with the CMV immediate-early gene enhancer/promoter.

Additionally, DNA binding sites for eukaryotic transcription factors may be included in the vaccine vector. These sites allow transcription factors such as NF-κB and AP-2 to bind to the vector, allowing the nuclear location signal to mediate import of the vector to the nucleus.

A nucleic acid vaccine vector of the invention may also be resistant to eukaryotic nuclease attack. In particular, the polyadenalytion signal may be modified to increase resistance to nuclease attack. Suitable polyadenylation signals that are resistanct to nuclease attack are known in the art. For instance, the SV40 late poly A signal may be used. Alternatively, other poly A adenylation signal sequences could be derived from other DNA viruses known to be successful in infecting avian and/or mammalian species.

A bacterium comprising a nucleic acid vaccine vector may also comprise a mutation that eliminates the periplasmic endonuclease I enzyme, such as a ΔendA mutation. This type of mutation is designed to increase vector survival upon the vector's release into the host cell.

### (d) Attenuation

In each of the above embodiments, a recombinant bacterium of the invention may also be attenuated. “Attenuated” refers to the state of the bacterium wherein the bacterium has been weakened from its wild-type fitness by some form of recombinant or physical manipulation. This includes altering the genotype of the bacterium to reduce its ability to cause disease. However, the bacterium's ability to colonize the gastrointestinal tract (in the case of Salmonella) and induce immune responses is, preferably, not substantially compromised. For instance, in one embodiment, regulated attenuation allows the recombinant bacterium to express one or more nucleic acids encoding products important for the bacterium to withstand stresses encountered in the host after immunization. This allows efficient invasion and colonization of lymphoid tissues before the recombinant bacterium is regulated to display the attenuated phenotype.

In one embodiment, a recombinant bacterium may be attenuated by regulating LPS O-antigen. In other embodiments, attenuation may be accomplished by altering (e.g., deleting) native nucleic acid sequences found in the wild type bacterium. For instance, if the bacterium is Salmonella, non-limiting examples of nucleic acid sequences which may be used for attenuation include: a pab nucleic acid sequence, a pur nucleic acid sequence, an aro nucleic acid sequence, asdA, a dap nucleic acid sequence, nadA, pncB, galE, pmi, fur, rpsL, ompR, htrA, hemA, cdt, cya, crp, dam, phoP, phoQ, rfc, poxA, galU, mviA, sodC, recA, ssrA, sirA, inv, hilA, rpoE, flgM, tonB, slyA, and any combination thereof. Exemplary attenuating mutations may be aroA, aroC, aroD, cdt, cya, crp, phoP, phoQ, ompR, galE, and htrA.

In certain embodiments, the above nucleic acid sequences may be placed under the control of a sugar regulated promoter wherein the sugar is present during in vitro growth of the recombinant bacterium, but substantially absent within an animal or human host. The cessation in transcription of the nucleic acid sequences listed above would then result in attenuation and the inability of the recombinant bacterium to induce disease symptoms.

The bacterium may also be modified to create a balanced-lethal host-vector system, although other types of systems may also be used (e.g., creating complementation heterozygotes). For the balanced-lethal host-vector system, the bacterium may be modified by manipulating its ability to synthesize various essential constituents needed for synthesis of the rigid peptidoglycan layer of its cell wall. In one example, the constituent is diaminopimelic acid (DAP). Various enzymes are involved in the eventual synthesis of DAP. In one example, the bacterium is modified by using a ΔasdA mutation to eliminate the bacterium's ability to produce β-aspartate semialdehyde dehydrogenase, an enzyme essential for the synthesis of DAP. One of skill in the art can also use the teachings of U.S. Pat. No. 6,872,547 for other types of mutations of nucleic acid sequences that result in the abolition of the synthesis of DAP. These nucleic acid sequences may include, but are not limited to, dapA, dapB, dapC, dapD, dapE, dapF, and asdA. Other modifications that may be employed include modifications to a bacterium's ability to synthesize D-alanine or to synthesize D-glutamic acid (e.g., Δmurl mutations), which are both unique constituents of the peptidoglycan layer of the bacterial cell wall

Yet another balanced-lethal host-vector system comprises modifying the bacterium such that the synthesis of an essential constituent of the rigid layer of the bacterial cell wall is dependent on a nutrient (e.g., arabinose) that can be supplied during the growth of the microorganism. For example, a bacterium may comprise the ΔP_{murA}:TT araC P_{BAD} murA deletion-insertion mutation. This type of mutation makes synthesis of muramic acid (another unique essential constituent of the peptidoglycan layer of the bacterial cell wall) dependent on the presence of arabinose that can be supplied during growth of the bacterium in vitro.

Other means of attenuation are known in the art.

i. Regulated Attenuation

The present invention also encompasses a recombinant bacterium capable of regulated attenuation. Generally speaking, the bacterium comprises a chromosomally integrated regulatable promoter. The promoter replaces the native promoter of, and is operably linked to, at least one nucleic acid sequence encoding an attenuation protein, such that the absence of the function of the protein renders the bacterium attenuated. In some embodiments, the promoter is modified to optimize the regulated attenuation.

In each of the above embodiments described herein, more than one method of attenuation may be used. For instance, a recombinant bacterium of the invention may comprise a regulatable promoter chromosomally integrated so as to replace the native promoter of, and be operably linked to, at least one nucleic acid sequence encoding an attenuation protein, such that the absence of the function of the protein renders the bacterium attenuated, and the bacterium may comprise another method of attenuation detailed in section I above.

### A. Attenuation Protein

Herein, “attenuation protein” is meant to be used in its broadest sense to encompass any protein the absence of which attenuates a bacterium. For instance, in some embodiments, an attenuation protein may be a protein that helps protect a bacterium from stresses encountered in the gastrointestinal tract or respiratory tract. Non-limiting examples may be the RpoS, PhoPQ, OmpR, Fur, and Crp proteins. In other embodiments, the protein may be necessary to synthesize a component of the cell wall of the bacterium, or may itself be a necessary component of the cell wall such as the protein encoded by murA. In still other embodiments, the protein may be listed in Section i above.

The native promoter of at least one, two, three, four, five, or more than five attenuation proteins may be replaced by a regulatable promoter as described herein. In one embodiment, the promoter of one of the proteins selected from the group comprising RpoS, PhoPQ, OmpR, Fur, and Crp may be replaced. In another embodiment, the promoter of two, three, four or five of the proteins selected from the group comprising RpoS, PhoPQ, OmpR, Fur, and Crp may be replaced.

If the promoter of more than one attenuation protein is replaced, each promoter may be replaced with a regulatable promoter, such that the expression of each attenuation protein encoding sequence is regulated by the same compound or condition. Alternatively, each promoter may be replaced with a different regulatable promoter, such that the expression of each attenuation protein encoding sequence is regulated by a different compound or condition such as by the sugars arabinose, maltose, rhamnose or xylose.

### B. Regulatable Promoter

The native promoter of a nucleic acid sequence encoding an attenuation protein is replaced with a regulatable promoter operably linked to the nucleic acid sequence encoding an attenuation protein. The term “operably linked,” is defined above.

The regulatable promoter used herein generally allows transcription of the nucleic acid sequence encoding the attenuation protein while in a permissive environment (i.e. in vitro growth), but cease transcription of the nucleic acid sequence encoding an attenuation protein while in a non-permissive environment (i.e. during growth of the bacterium in an animal or human host). For instance, the promoter may be responsive to a physical or chemical difference between the permissive and non-permissive environment. Suitable examples of such regulatable promoters are known in the art and detailed above.

In some embodiments, the promoter may be responsive to the level of arabinose in the environment, as described above. In other embodiments, the promoter may be responsive to the level of maltose, rhamnose, or xylose in the environment, as described above. The promoters detailed herein are known in the art, and methods of operably linking them to a nucleic acid sequence encoding an attenuation protein are known in the art.

In certain embodiments, a recombinant bacterium of the invention may comprise any of the following: ΔP_{fur}::TT araC P_{BAD} fur, ΔP_{crp}::TT araC P_{BAD} crp, ΔP_{phoPQ}::TT araC P_{BAD} phoPQ, or a combination thereof. Growth of such strains in the presence of arabinose leads to transcription of the fur, phoPQ, and/or crp nucleic acid sequences, but nucleic acid sequence expression ceases in a host because there is no free arabinose. Attenuation develops as the products of the fur, phoPQ, and/or the crp nucleic acid sequences are diluted at each cell division. Strains with the ΔP_{fur} and/or the ΔP_{phoPQ} mutations are attenuated at oral doses of 10⁹ CFU, even in three-week old mice at weaning. Generally speaking, the concentration of arabinose necessary to induce expression is typically less than about 2%. In some embodiments, the concentration is less than about 1.5%, 1%, 0.5%, 0.2%, 0.1%, or 0.05%. In certain embodiments, the concentration may be about 0.04%, 0.03%, 0.02%, or 0.01%. In an exemplary embodiment, the concentration is about 0.05%. Higher concentrations of arabinose or other sugars may lead to acid production during growth that may inhibit desirable cell densities. The inclusion of mutations such as ΔaraBAD or mutations that block the uptake and/or breakdown of maltose, rhamnose, or xylose, however, may prevent such acid production and enable use of higher sugar concentrations with no ill effects.

When the regulatable promoter is responsive to arabinose, the onset of attenuation may be delayed by including additional mutations, such as ΔaraBAD23, which prevents use of arabinose retained in the cell cytoplasm at the time of oral immunization, and/or ΔaraE25 that enhances retention of arabinose. Thus, inclusion of these mutations may be beneficial in at least two ways: first, enabling higher culture densities, and second enabling a further delay in the display of the attenuated phenotype that may result in higher densities in effector lymphoid tissues to further enhance immunogenicity.

### C. Modifications

Attenuation of the recombinant bacterium may be optimized by modifying the nucleic acid sequence encoding an attenuation protein and/or promoter. Methods of modifying a promoter and/or a nucleic acid sequence encoding an attenuation protein are the same as those detailed above with respect to repressors in section (e).

In some embodiments, more than one modification may be performed to optimize the attenuation of the bacterium. For instance, at least one, two, three, four, five, six, seven, eight or nine modifications may be performed to optimize the attenuation of the bacterium. In various exemplary embodiments of the invention, the SD sequences and/or the start codons for the fur and/or the phoPQ virulence nucleic acid sequences may be altered so that the production levels of these nucleic acid products are optimal for regulated attenuation.

### (e) Other Mutations

A bacterium may further comprise additional mutations. Such mutations may include the regulation of serotype-specific antigens, those detailed below.

i. Regulated Expression of a Nucleic Acid Sequence Encoding at Least One Serotype-Specific Antigen

Generally speaking, a recombinant bacterium of the invention is capable of the regulated expression of a nucleic acid sequence encoding at least one serotype-specific antigen. As used herein, the phrase “serotype-specific antigen” refers to an antigen that elicits an immune response specific for the bacterial vector serotype. In some embodiments, the immune response to a serotype-specific antigen may also recognize closely related strains in the same serogroup, but in a different, but related, serotype. Non-limiting examples of serotype-specific antigens may include LPS O-antigen, one or more components of a flagellum, and Vi capsular antigen. In some embodiments, the expression of at least one, at least two, at least three, or at least four nucleic acid sequences encoding a serotype-specific antigen are regulated in a bacterium of the invention.

The phrase “regulated expression of a nucleic acid encoding at least one serotype-specific antigen” refers to expression of the nucleic acid sequence encoding a serotype-antigen such that the bacterium does not substantially induce an immune response specific to the bacterial vector serotype. In one embodiment, the expression of the serotype-specific antigen is eliminated. In another embodiment, the expression is substantially reduced. In yet another embodiment, the expression of the serotype-specific antigen is reduced in a temporally controlled manner. For instance, the expression of the serotype-specific antigen may be reduced during growth of the bacterium in a host, but not during in vitro growth.

The expression of a nucleic acid sequence encoding a Salmonella serotype-specific antigen may be measured using standard molecular biology and protein and carbohydrate chemistry techniques known to one of skill in the art. As used herein, “substantial reduction” of the expression of a nucleic acid sequence encoding a serotype-specific antigen refers to a reduction of at least about 1% to at least about 99.9% as compared to a Salmonella bacterium in which no attempts have been made to reduce serotype-specific antigen expression. In one embodiment, the expression of a nucleic acid sequence encoding a serotype-specific antigen is reduced by 100% by using a deletion mutation. In other embodiments of the invention, the expression of a nucleic acid sequence encoding a serotype-specific antigen is reduced by at least about 99.9%, 99.5%, 99%, 98%, 97%, 96%, 95%, 94%, 93%, 92%, 91% or 90%. In yet other embodiments of the invention, the expression of a nucleic acid sequence encoding a serotype-specific antigen is reduced by at least about 89%, 88%, 87%, 86%, 85%, 84%, 83%, 82%, 81% or 80%. In still other embodiments of the invention, the expression of a nucleic acid sequence encoding a serotype-specific antigen is reduced by at least about 75%, 70%, 65%, 60%, 55%, or 50%. In additional embodiments, the expression of a nucleic acid sequence encoding a serotype-specific antigen is reduced by at least about 45%, 40%, 35%, 30%, 25%, or 20%. In yet additional embodiments, the expression of a nucleic acid sequence encoding a serotype-specific antigen is reduced by at least about 15%, 10%, 5%, 4%, 3%, 2% or 1%.

Methods of regulating expression of a nucleic acid sequence encoding at least one serotype-specific antigen are discussed in detail below, and in the examples.

### A. Regulating the Expression of a Nucleic Acid Sequence Encoding LPS O-Antigen

In one embodiment, the expression of a nucleic acid sequence encoding the serotype-specific antigen LPS O-antigen is regulated by mutating the pmi nucleic acid sequence, which encodes a phosphomannose isomerase needed for the bacterium to interconvert fructose-6-P and mannose-6-P. In some instances, the bacterium comprises a Δpmi mutation, such as a Δpmi-2426 mutation. A bacterium comprising a Δpmi-2426 mutation, grown in the presence of mannose, is capable of synthesizing a complete LPS O-antigen. But non-phosphorylated mannose, which is the form required for bacterial uptake, is unavailable in vivo. Hence, a bacterium comprising a Δpmi-2426 mutation loses the ability to synthesize LPS O-antigen serotype specific side chains after a few generations of growth in vivo. The LPS that is synthesized comprises a core structure that is substantially similar across many diverse Salmonella serotypes. This results in a bacterium that is capable of eliciting an immune response against at least two Salmonella serotypes without substantially inducing an immune response specific to the serotype of the bacterial vector.

A bacterium of the invention that comprises a Δpmi mutation may also comprise other mutations that ensure that mannose available to the bacterium during in vitro growth is used for LPS O-antigen synthesis. For instance, a bacterium may comprise a Δ(gmd-fcl)-26 mutation. This mutation deletes two nucleic acid sequences that encode enzymes for conversion of GDP-mannose to GDP-fucose. This ensures that mannose available to the bacterium during in vitro growth is used for LPS O-antigen synthesis and not colanic acid production. Similarly, a bacterium may comprise the Δ(wcaM-wza)-8 mutation, which deletes all 19 nucleic acid sequences necessary for colanic acid production, and also precludes conversion of GDP-mannose to GDP-fucose.

In addition to regulating LPS O-antigen synthesis with mannose, the synthesis of LPS O-antigen may be regulated by arabinose, which is also absent in vivo. For instance, a bacterium may comprise the mutation ΔP_{rfc}::TT araC P_{BAD} rfc. (P stands for promoter and TT stands for transcription terminator.) The rfc nucleic acid sequence is necessary for the addition of O-antigen subunits, which typically comprise three or four sugars, in a repeat fashion. When the rfc nucleic acid sequence is absent, only one O-antigen repeat subunit is added to the LPS core polysaccharide. Normally, the serotype-specific O-antigen contains some 50 or so repeats of the O-antigen subunit, catalyzed by the enzyme encoded by the rfc nucleic acid sequence. In the case of a bacterium comprising the ΔP_{rfc}::TT araC P_{BAD} rfc deletion-insertion mutation, expression of the rfc nucleic acid sequence is dependant on the presence of arabinose that can be supplied during in vitro growth of the strain, but that is absent in vivo. Consequently, rfc expression ceases in vivo, resulting in the cessation of assembly of the O-antigen repeat structure. This reduces the bacterium's ability to induce an immune response against the serotype-specific O-antigen.

Another means to regulate LPS O-antigen expression is to eliminate the function of galE in a recombinant bacterium of the invention. The galE nucleic acid sequence encodes an enzyme for the synthesis of UDP-Gal, which is a substrate for LPS O-antigen, the outer LPS core and colanic acid. Growth of a bacterium comprising a suitable galE mutation in the presence of galactose leads to the synthesis of O-antigen and the LPS core. Non-phosphorylated galactose is unavailable in vivo, however, and in vivo synthesis of UDP-Gal ceases, as does synthesis of the O-antigen and the LPS outer core. One example of a suitable galE mutation is the Δ(galE-ybhC)-851 mutation.

In certain embodiments, a bacterium of the invention may comprise one or more of the Δpmi, ΔP_{rfc}::TT araC P_{BAD} rfc, and ΔgalE mutations, with or without a Δ(gmd-fcl)-26 or Δ(wcaM-wza)-8 mutation. Such a combination may yield a recombinant bacterium that synthesizes all components of the LPS core and O-antigen side chains when grown in vitro (i.e. in the presence of suitable concentrations of mannose, arabinose and galactose), but that ceases to synthesize the LPS outer core and O-antigen in vivo due to the unavailability of free unphosphorylated mannose, arabinose or galactose. Also, a recombinant bacterium with the inability to synthesize the LPS outer core and/or O-antigen is attenuated, as the bacterium is more susceptible to killing by macrophages and/or complement-mediated cytotoxicity. Additionally, a bacterium with the inability to synthesize the LPS outer core and O-antigen in vivo, induces only a minimal immune response to the serotype-specific LPS O-antigen.

The regulated expression of one or more nucleic acids that enable synthesis of LPS O-antigen allows a recombinant bacterium of the invention to be supplied with required sugars such as mannose, arabinose and/or galactose during in vitro growth of the bacterium, ensuring complete synthesis of the LPS O-antigen. This is important, because the presence of the O-antigen on the recombinant bacterium cell surface is indispensable for the strain to invade and colonize lymphoid tissue, a necessary prerequisite for being immunogenic. In vivo, LPS O-antigen synthesis ceases due to the unavailability of the free unphosphorylated sugars. Consequently, the recombinant bacterium is attenuated, becoming more susceptible to complement-mediated cytotoxicity and macrophage phagocytosis. Also, when LPS O-antigen synthesis ceases, the LPS core is exposed. The core is a cross-reactive antigen with a similar structure in all Salmonella serotypes. In addition, when LPS O-antigen synthesis ceases, any cross-reactive outer membrane proteins expressed by the recombinant bacterium are exposed for surveillance by the host immune system.

### B. The Expression of a Nucleic Acid Sequence Encoding a Component of a Flagellum

In one embodiment, the expression of a nucleic acid encoding a serotype-specific component of a flagellum is regulated by mutating the nucleic acid that encodes FljB or FliC. For instance, a bacterium of the invention may comprise a ΔfljB217 mutation. Alternatively, a bacterium may comprise a ΔfliC180 mutation. The ΔfljB217 mutation deletes the structural nucleic acid sequence that encodes the Phase II flagellar antigen whereas the ΔfliC180 mutation deletes the 180 amino acids encoding the antigenically variable serotype-specific domain of the Phase I FliC flagellar antigen. The portion of the flagellar protein that interacts with TLR5 to recruit/stimulate innate immune responses represents the conserved N- and C-terminal regions of the flagellar proteins and this is retained and expressed by strains with the ΔfliC180 mutation. In addition, the ΔfliC180 mutation retains the CD4-dependent T-cell epitope. It should be noted, that expression of the Phase I flagellar antigen and not the Phase II flagellar antigen potentiates S. Typhimurium infection of mice. S. Typhimurium recombinant bacteria with the Δpmi-2426, ΔfljB217 and ΔfliC180 mutations, when grown in the absence of mannose, are not agglutinated with antisera specific for the B-group O-antigen or the S. Typhimurium specific anti-flagellar sera. These recombinant bacteria are also non-motile since the FliC180 protein that is synthesized at high levels is not efficiently incorporated into flagella. When these recombinant bacteria are evaluated using HEK293 cells specifically expressing TLR5, the level of NF-κB production is about 50% higher than when using a ΔfliB217 F1iC⁺ strain that assembles flagellin into flagella and exhibits motility (there is no NF-κB production by the control ΔfljB217 ΔfliC2426 strain with no flagella). Similarly, recombinant bacteria with the Δ(galE-ybhC)-851, ΔfljB217 and ΔfliC180 mutations, when grown in the absence of galactose, are not agglutinated with antisera specific for the B-group O-antigen or the S. Typhimurium specific anti-flagellar sera. These bacteria are also non-motile.

In some embodiments, a bacterium may comprise both a ΔfljB217 and a ΔfliC2426 mutation. Such a bacterium will typically not synthesize flagella, and hence, will not be motile. This precludes interaction with TLR5 and up-regulation of NF-κB production. Such a bacterium will reduce bacterial-induced host programmed cell death.

### C. The Expression of a Nucleic Acid Sequence Encoding the Vi Capsular Antigen

Certain Salmonella strains, such as S. Typhi and S. Dublin, express the Vi capsular antigen. This antigen is serotype-specific, inhibits invasion, and acts to suppress induction of a protective immune response. Consequently, when a recombinant bacterium of the invention is derived from a strain comprising the Vi capsular antigen, one or more nucleic acids encoding the Vi capsular antigen will be deleted such that the Vi capsular antigen is not synthesized. Even though synthesis and display of the Vi capsular antigen on the Salmonella cell surface interferes with invasion and suppresses induction of immunity, the purified Vi antigen can be used as a vaccine to induce protective immunity against infection by Vi antigen displaying S. Typhi and S. Dublin strains.

ii. Eliciting an Immune Response Against at Least Two Salmonella Serotypes

A recombinant bacterium of the invention may be capable of eliciting an immune response against at least two Salmonella serotypes. This may be accomplished, for instance, by eliminating the serotype-specific LPS O-antigen side chains as discussed above. The remaining LPS core will elicit an immune response, inducing the production of antibodies against the LPS core. Since this LPS core is substantially identical in the several thousand Salmonella enterica serotypes, the antibodies potentially provide immunity against several diverse Salmonella enterica serotypes, such as Typhimurium, Heidelberg, Newport, Infantis, Dublin, Virchow, Typhi, Enteritidis, Berta, Seftenberg, Ohio, Agona, Braenderup, Hadar, Kentucky, Thompson, Montevideo, Mbandaka, Javiana, Oranienburg, Anatum, Paratyphi A, Schwarzengrund, Saintpaul, and Munchen.

In addition, the elimination of the LPS O-antigen provides the host immune system with better access to the outer membrane proteins of the recombinant bacterium, thereby enhancing induction of immune responses against these outer membrane proteins. In some embodiments, as described below, the outer membrane proteins may be upregulated to further enhance host immune responses to these proteins. Non-limiting examples of outer 0.0.

proteins include proteins involved in iron and manganese uptake, as described below. Iron and manganese are essential nutrients for enteric pathogens and the induction of antibodies that inhibit iron and manganese uptake in effect starves the pathogens, conferring protective immunity on the host. Additionally, since these proteins are homologous among the enteric bacteria, such host immune responses provide immunity against multiple Salmonella enterica serotypes as well as to other enteric bacterial pathogens such as strains of Yersinia, Shigella and Escherichia. As evidence of this, the attenuated S. Typhimurium vaccine vector strain not expressing any Yersinia antigen is able to induce significant protective immunity to high doses of orally administered Y. pseudotuberculosis.

The elicited immune response may include, but is not limited to, an innate immune response, a mucosal immune response, a humoral immune response and a cell-mediated immune response. In one embodiment, Th2-dependent mucosal and systemic antibody responses to the enteric antigen(s) are observed. Immune responses may be measured by standard immunological assays known to one of skill in the art. In an exemplary embodiment, the immune response is protective.

iii. Reduction in Fluid Secretion

In some embodiments, a recombinant bacterium of the invention may be modified so as to reduce fluid secretion in the host. For instance, the bacterium may comprise the ΔsopB1925 mutation. Alternatively, the bacterium may comprise the ΔmsbB48 mutation. In another alternative, the bacterium may comprise both the ΔsopB1925 mutation and the ΔmsbB48 mutation

iv. Biological Containment

Under certain embodiments, a live recombinant bacterium may possess the potential to survive and multiply if excreted from a host. This leads to the possibility that individuals not electing to be immunized may be exposed to the recombinant bacterium. Consequently, in certain embodiments, a recombinant bacterium of the invention may comprise one or more mutations that decrease, if not preclude, the ability of Salmonella vaccines to persist in the GI tract of animals.

In another embodiment, a recombinant bacterium of the invention may comprise one or more of the Δ(gmd fcl)-26 or Δ(wcaM-wza)-7, ΔagfBAC811, Δ(P_{agfD} agfG)-4, Δ(agfC-agfG)-999, ΔbcsABZC2118 or ΔbcsEFG2319 and Δ(yshA-yihW)-157 mutations that block synthesis of colanic acid, thin aggregative fimbriae (i.e., curli), cellulose and extracellular polysaccharide, respectively, all of which contribute to biofilm formation. In addition, the mutation ΔyhiR36 that prevents use of DNA as a nutrient, Δ(shdA-ratB)-64, ΔmisL2 and ΔbigA3 that encode four proteins that enable Salmonella to adhere to host extracellular matrix proteins and ΔackA233 that blocks use of acetate, may be used as a means for biological containment. Likewise, inclusion of mutations that block use of the sugars fucose and ribose such as ΔfucOR8 and Δrbs-19 will reduce ability of vaccine strains to persist in the intestinal tract. In exemplary embodiments, a recombinant bacterium comprising a biological containment mutation is not adversely affected in its virulence.

In some embodiments, the recombinant bacterium may comprise a method of regulated delayed lysis in vivo that prevents bacterial persistence in vivo and survival if excreted. These chromosomal mutations may include: Δ(gmd fcl)-26 or Δ(wcaM-wza)-8 that precludes synthesis of colanic acid that can protect cells undergoing cell wall-less death from lysing completely, ΔagfBAC811 and Δ(agfC-agfG)-999 that block synthesis of thin aggregative fimbriae (curli) that are critical for biofilm formation to enable persistent colonization on bile stones in the gall bladder, ΔasdA27::TT araC P_{BAD} c2 insertion-deletion mutation to impose a requirement for the peptidoglycan constituent DAP and ΔP_{murA12}::TTaraC P_{BAD} murA or the improved ΔP_{murA25}::TTaraC P_{BAD} murA insertion-deletion mutation as a conditional-lethal mutation blocking synthesis of the peptidoglycan constituent muramic acid. The latter two mutations are typically complemented by a regulated delayed lysis plasmid vector such as pYA3681 or the improved pYA4763 that has an arabinose-dependent expression of asdA and murA genes. A recombinant bacterium comprising such mutations grows normally in the presence of arabinose. In vivo, however, the bacterium ceases to express any nucleic acids encoding the Asd and MurA enzymes, such that synthesis of the peptidoglycan cell wall layer ceases, ultimately resulting in the lysis of the bacterium. This lysis may result in the release of a bolus of antigen specific for an enteric pathogen, thereby serving as a means to enhance induction of immunity against that enteric pathogen while conferring biological containment.

In some embodiments, a recombinant bacterium may comprise a mutation that blocks the recycling of cell wall peptidoglycan to ensure lysis occurs. For instance, a bacterium may comprise an ampG mutation, an ampD mutation or a nagE mutation, or two or three of these mutations.

### v. crp Cassette

In some embodiments, a recombinant bacterium of the invention may also comprise a ΔP_{crp}::TT araC P_{BAD} crp deletion-insertion mutation. Since the araC P_{BAD} cassette is dependent both on the presence of arabinose and the binding of the catabolite repressor protein Crp, a ΔP_{crp}::TT araC P_{BAD} crp deletion-insertion mutation may be included as an additional means to reduce expression of any nucleic acid sequence under the control of the P_{BAD} promoter. This means that when the bacterium is grown in a non-permissive environment (i.e. no arabinose) both the repressor itself and the Crp protein cease to be synthesized, consequently eliminating both regulating signals for the araC P_{BAD} regulated nucleic acid sequence. This double shut off of araC P_{BAD} may constitute an additional safety feature ensuring the genetic stability of the desired phenotypes and in the case of strains with the regulated delayed lysis phenotype provides an additional means to preclude synthesis of the Asd and MurA enzymes and ensure lysis in vivo.

Generally speaking, the activity of the Crp protein requires interaction with cAMP, but the addition of glucose, which may inhibit synthesis of cAMP, decreases the ability of the Crp protein to regulate transcription from the araC P_{BAD} promoter. Consequently, to avoid the effect of glucose on cAMP, glucose may be substantially excluded from the growth media, or variants of crp may be isolated or constructed that synthesize a Crp protein that is not dependent on cAMP to regulate transcription from P_{BAD}. Two such alterations in the crp gene have been made with amino acid substitution mutations T1271, Q170K and L195R to result in the crp-70 gene modification and with amino acid substitutions I112L, T1271 and A144T to result in the crp-72 gene modification. Both constructions have been made with araC P_{BAD} to yield the ΔP_{crp70}::TT araC P_{BAD} crp-70 and ΔP_{crp72}::TT araC P_{BAD} crp-72 deletion-insertion mutations. In both cases, synthesis of the Crp protein induced by arabinose is insensitive to the addition of glucose. This strategy may also be used in other systems responsive to Crp, such as the systems responsive to rhamnose, xylose and maltose described above.

vi. Hyper-Invasiveness

A recombinant bacterium of the invention may also be hyper-invasive. As used herein, “hyper-invasive” refers to a bacterium that can invade a host cell more efficiently than a wild-type bacterium of the same strain. Invasion may be determined by methods known in the art, e.g. CFUs/g of tissue. In some embodiments, a recombinant bacterium may be capable of increased invasion of M cells. Generally speaking, such a bacterium may comprise a mutation that increases expression of hilA. For instance, the promoter of hilA may be mutated to enable constitutive expression of hilA. A non-limiting example may include a ΔP_{hilA}::P_{trcΔlacO} hilA mutation, such as ΔP_{hilA}:P_{trcΔlacO888} hilA. Such a mutation replaces the wild-type hilA promoter with the P_{trc} promoter that lacks the lacO operator sequence. This allows constitutive expression of hilA, even when lacI is expressed. Alternatively, deletion of the lrp nucleic acid sequence may be used to increase hilA expression.

vii. Reduced Bacterium-Induced Host Programmed Cell Death

Programmed cell death of a host cell invaded by a bacterium of the invention is likely to diminish the transcription of a nucleic acid sequence comprising a nucleic acid vaccine vector delivered by the bacterium. Consequently, in some embodiments, a recombinant bacterium of the invention may be capable of reducing bacterium-induced host programmed cell death compared to a wild-type bacterium of the same strain. Non-limiting examples of bacterium-induced host programmed cell death may include apoptosis and pyroptosis. Methods of detecting and measuring bacterium-induced host programmed cell death are known in the art.

In one embodiment, a bacterium of the invention capable of reducing bacterium-induced host programmed cell death may comprise a mutation affecting the pathway inducing apoptosis/pyroptosis. Non-limiting examples of such a mutation may include mutations in a deubiquitinase nucleic acid sequence, such as sseL, and/or mutations in a temperature-sensing protein nucleic acid sequence, such as tlpA. For instance, a bacterium may comprise a ΔsseL mutation, a ΔtlpA mutation, or both mutations. In another embodiment, a bacterium may completely lack flagella.

### (f) Exemplary Bacterium

In an exemplary embodiment, a bacterium may comprise one or more mutations to allow endosomal escape (section (a) above), one or more mutations to induce lysis of the bacterium (section (b) above), one or more mutations to express a nucleic acid encoding an antigen (section (c) above), one or more mutations to attenuate the bacterium (section (d) above), and one or more mutations to enhance the performance of the bacterium as a vaccine (section (e) above).

### II. Vaccine Compositions and Administration

A recombinant bacterium of the invention may be administered to a host as a vaccine composition. As used herein, a vaccine composition is a composition designed to elicit an immune response to the recombinant bacterium, including any antigens that may be synthesized by the bacterium. In an exemplary embodiment, the immune response is protective, as described above. In another exemplary embodiment, the immune response is a cellular immune response. In yet another exemplary embodiment, the immune response is a Th1 response. Immune responses to antigens are well studied and widely reported. A survey of immunology is given by Paul, W E, Stites D et. al. and Ogra P L. et. al. Mucosal immunity is also described by Ogra P L et. al.

Vaccine compositions of the present invention may be administered to any host capable of mounting an immune response. Such hosts may include all vertebrates, for example, mammals, including domestic animals, agricultural animals, laboratory animals, and humans, and various species of birds, including domestic birds and birds of agricultural importance. Preferably, the host is a warm-blooded animal. The vaccine can be administered as a prophylactic or for treatment purposes.

In exemplary embodiments, the recombinant bacterium is alive when administered to a host in a vaccine composition of the invention. Suitable vaccine composition formulations and methods of administration are detailed below.

### (a) Vaccine Composition

A vaccine composition comprising a recombinant bacterium of the invention may optionally comprise one or more possible additives, such as carriers, preservatives, stabilizers, adjuvants, and other substances.

In one embodiment, the vaccine comprises an adjuvant. Adjuvants, such as aluminum hydroxide or aluminum phosphate, are optionally added to increase the ability of the vaccine to trigger, enhance, or prolong an immune response. In exemplary embodiments, the use of a live attenuated recombinant bacterium may act as a natural adjuvant. The vaccine compositions may further comprise additional components known in the art to improve the immune response to a vaccine, such as T cell co-stimulatory molecules or antibodies, such as anti-CTLA4. Additional materials, such as cytokines, chemokines, bacterial nucleic acid sequences naturally found in bacteria, like CpG, and adjuvants compatible with live bacterial vaccines such as Montamide Gel 01, IMS1312, IMS1313 and ISA 201, are also potential vaccine adjuvants.

In another embodiment, the vaccine may comprise a pharmaceutical carrier (or excipient). Such a carrier may be any solvent or solid material for encapsulation that is non-toxic to the inoculated host and compatible with the recombinant bacterium. A carrier may give form or consistency, or act as a diluent. Suitable pharmaceutical carriers may include liquid carriers, such as normal saline and other non-toxic salts at or near physiological concentrations, and solid carriers not used for humans, such as talc or sucrose, or animal feed. Carriers may also include stabilizing agents, wetting and emulsifying agents, salts for varying osmolarity, encapsulating agents, buffers, and skin penetration enhancers. Carriers and excipients as well as formulations for parenteral and nonparenteral drug delivery are set forth in Remington's Pharmaceutical Sciences 19th Ed. Mack Publishing (1995). When used for administering via the bronchial tubes, the vaccine is preferably presented in the form of an aerosol.

Care should be taken when using additives so that the live recombinant bacterium is not killed, or have its ability to effectively colonize lymphoid tissues such as the GALT, NALT and BALT compromised by the use of additives. Stabilizers, such as lactose or monosodium glutamate (MSG), may be added to stabilize the vaccine formulation against a variety of conditions, such as temperature variations or a freeze-drying process.

The dosages of a vaccine composition of the invention can and will vary depending on the recombinant bacterium, the regulated antigen, and the intended host, as will be appreciated by one of skill in the art. Generally speaking, the dosage need only be sufficient to elicit a protective immune response in a majority of hosts. Routine experimentation may readily establish the required dosage. Typical initial dosages of vaccine for oral administration could be about 1×10⁷ to 1×10¹⁰ CFU depending upon the age of the host to be immunized. Administering multiple dosages may also be used as needed to provide the desired level of protective immunity.

### (b) Methods of Administration

In order to stimulate a preferred response of the GALT, NALT or BALT cells, administration of the vaccine composition directly into the gut, nasopharynx, or bronchus is preferred, such as by oral administration, intranasal administration, gastric intubation or in the form of aerosols, although other methods of administering the recombinant bacterium, such as intravenous, intramuscular, subcutaneous injection or other parenteral routes, are possible.

In some embodiments, these compositions are formulated for administration by injection (e.g., intraperitoneally, intravenously, subcutaneously, intramuscularly, etc.). Accordingly, these compositions are preferably combined with pharmaceutically acceptable vehicles such as saline, Ringer's solution, dextrose solution, and the like.

### III. Kits

The invention also encompasses kits comprising any one of the compositions above in a suitable aliquot for vaccinating a host in need thereof. In one embodiment, the kit further comprises instructions for use. In other embodiments, the composition is lyophilized such that addition of a hydrating agent (e.g., buffered saline) reconstitutes the composition to generate a vaccine composition ready to administer, preferably orally.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### IV. Methods of Use

A further aspect of the invention encompasses methods of using a recombinant bacterium of the invention. For instance, in one embodiment the invention provides a method for modulating a host's immune system. The method comprises administering to the host an effective amount of a composition comprising a recombinant bacterium of the invention. One of skill in the art will appreciate that an effective amount of a composition is an amount that will generate the desired immune response (e.g., cellular). Methods of monitoring a host's immune response are well-known to physicians and other skilled practitioners. For instance, assays such as ELISA, and ELISPOT may be used. Effectiveness may be determined by monitoring the amount of the antigen of interest remaining in the host, or by measuring a decrease in disease incidence caused by a given pathogen in a host. For certain pathogens, cultures or swabs taken as biological samples from a host may be used to monitor the existence or amount of pathogen in the individual.

In another embodiment, the invention provides a method for eliciting a cellular immune response against an antigen in a host. The method comprises administering to the host an effective amount of a composition comprising a recombinant bacterium of the invention.

In still another embodiment, a recombinant bacterium of the invention may be used in a method for eliciting a cellular immune response against a pathogen in an individual in need thereof. The method comprises administrating to the host an effective amount of a composition comprising a recombinant bacterium as described herein. In a further embodiment, a recombinant bacterium described herein may be used in a method for ameliorating one or more symptoms of an infectious disease in a host in need thereof. The method comprises administering an effective amount of a composition comprising a recombinant bacterium as described herein.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

The following examples illustrate various iterations of the invention.

### Introduction for Examples 1-5

Influenza remains one of the most significant disease worldwide causing acute respiratory illnesses and accounts for 25% of the infections that exacerbate chronic lung infections [1]. Several epidemics and three major pandemics have been reported. Influenza infections are primarily and effectively controlled by vaccines that elicit neutralizing antibodies against the surface proteins hemagglutinin (HA) and neuraminidase (NA). Influenza vaccines have to be reformulated annually to match the circulating strains due to antigenic drift and do not protect against strains that arise by antigenic shift due to reassortment of gene segments from different species. The most recent example of this is the emergence of pandemic swine (H1N1) flu in 2009 containing sequences from human, avian and both North American and Eurasian swine origins [2, 3].

Inactivated vaccines do not generally stimulate cellular immunity. There is much interest in development of a vaccine that elicits cellular immunity against the conserved proteins like the Influenza nucleoprotein (NP) to stimulate an efficient T cell response that would result in clearing viral infection. T cell epitopes in NP are well defined [4] and both CD8 and CD4 T cells play an important role in protection afforded by NP [5]. Several groups have delivered NP using adenovirus [6], vaccinia virus [7], as a purified immunogen [8, 9] or as a DNA vaccine [10]. These studies have demonstrated influenza specific T cell responses but shown moderate to low protection against virus challenge. DNA vaccination with NP protects against the homologous and comparatively low dose heterologous challenges in mice models [11-13].

Attenuated Salmonella vaccines have successfully been used as a vaccine carrier for several bacterial, viral and parasitic antigens [14]. Orally delivered vaccines have an advantage of inducing mucosal as well as systemic immune responses to numerous antigens as compared to vaccines delivered via parenteral routes [14] which is extremely important for an infectious agent like Influenza that gains entry through the mucosal surface. In addition, orally administered vaccines have the advantage of being cost effective since they eliminate the use of needles and syringes making it an affordable choice for mass vaccination. Attempts were made to deliver Influenza NP via recombinant Salmonella SL3261 (aroA mutant) in early 90's resulted in antigen specific CD4⁺ T cells but failed to elicit any CD8⁺ T cells or any protection in face of a viral challenge [15].

The inventors have successfully developed several recombinant attenuated Salmonella vaccines (RASVs) for infectious agents like Streptococcus pneumoniae, Yersinia, and Eimeria [16-18]. The RASV used in such studies are genetically modified for attenuation and rely on an Asd⁺ balanced-lethal host-vector system for plasmid maintenance that eliminates the need for antibiotic resistance markers [19]. Deletion of the asdA gene imposes an obligate requirement for diaminopimelic acid (DAP), an essential constituent of peptidoglycan, so the bacterium can't survive in vivo. The RASV strains are exquisitely designed to possess the attributes of a wild-type strain at the time of immunization that enable them to encounter stresses in gut associated lymphoid tissue (GALT) and successfully invade and colonize the lymphoid organs before attenuation sets in due to unavailability of inducers under in vivo conditions [20]. The success of vaccine strain is dependent on its ability to survive the host defenses with minimal damage to the host and on maximal synthesis of the delivered antigen at appropriate effector lymphoid sites. These goals were accomplished by designing a regulated delayed lysis system which is based on the deletion of the asdA gene and the arabinose regulated expression of murA as a means to confer attenuation after colonization of the lymphoid tissue and eventually results in cell lysis conferring biological containment. Release of antigen by programmed cell lysis results in a strong Th1 type antibody response [21]. However, the induction of T cell responses after vaccination with a vaccine comprising a regulated lysis system has not been evaluated so far.

A critical factor in the success of a RASV delivering an antigen from an intracellular pathogen like NP is the ability to deliver it directly to the cytosol or to produce it inside the cytosol of the cell so that it could be taken up for proteosomal degradation and presented in the context of MHC-I molecules. Salmonella invade the nonphagocytic cells like intestinal epithelium and remains inside the endosome in a structure called the Salmonella containing vacuole (SCV) and is directed to the endolysosomal pathway eventually presenting to MHC-II molecules [22].

Gram negative bacteria use a type III secretion system (T3SS), a syringe like structure for injection of effector proteins into the host cell cytosol. By fusing heterologous epitopes to the proteins that are secreted by T3SS, the epitopes can be delivered to the cytosol of the cell and presented efficiently by the MHC-I molecules [23, 24]. Several effector proteins like SopE and SptP have been described for efficient delivery of heterologous epitopes to the cytosol by Salmonella and have been used to effectively deliver Eimeria acervulina antigen EASZ240 and EAMZ-250 and Eimeria tenella antigen SO7 [25, 26].

Delivery of Influenza (NP 366-374) and lymphocytic choriomeningitis virus (LCMV) epitopes by fusion to bacterial SptP [27] and through Salmonella T3SS resulted in T cell responses and protection against a lethal challenge with LCMV [27]. Fusion of fragments of Simian immunodeficiency virus (SIV) Gag protein to SopE effector resulted in efficient priming of CD4⁺ and CD8⁺ responses [28]. Therefore, various NP fragments and epitopes were fused to SopE and delivered through RASV strains and T cell responses elicited and protection conferred by it against viral challenge were evaluated.

Another strategy to release Salmonella from the endosome after it has invaded the cells is by the deletion of the sifA gene. The sifA gene is a SPI-2 encoded, type III secreted effector protein that governs conversion of the Salmonella-containing vacuoles (SCV) into filaments and its deletion leads to escape of Salmonella into the cytosol [29]. Replication of S. Typhimurium inside the SCV alters the processing of SCV by the normal endocytic pathway [30]. Intravacuolar replication of bacteria takes place with the formation of sifs (Salmonella induced filaments) that connect the SCVs. Salmonella strains with a sifA mutation loose the integrity of the vacuolar membranes and are released in the cytoplasm of the cell. Salmonella strains with sifA mutations are attenuated but replicate more efficiently than the wild-type bacteria in the epithelial cells [30, 31]. The sifA gene was deleted in a regulated lysis RASV strain that permits Salmonella to exit the endosome immediately upon invasion into a host cell and more rapidly multiply in the cytoplasm (cytosol) to enable comparative studies on resulting cellular immune responses with or without the sifA deletion.

The following examples describe methods for delivering the known T cell epitopes and antigen to the cytosol of the cell for efficient T cell priming and presentation to MHC-I molecules by RASV strains. As a model, influenza NP was used as a target antigen. The TTSS with effector protein SopE was employed for stimulation of antigen specific T cells. As an alternative strategy, strains containing regulated lysis mutations with the sifA deletion were generated. Cellular and humoral immune responses and protection afforded by such vaccination against Influenza challenge were evaluated in mice. The examples below describe for the first time a novel bacterial gene delivery system for a viral antigen utilizing orally administered recombinant Salmonella vector that delivers the antigen to the cytosol and results in efficient CD8⁺ T cell priming and protection of mice against influenza challenge by decreasing mobidity and mortality. This also represents a means of delivering T-cell antigens to enable class I presentation to elicit CD8-dependent CTL responses.

### Materials and Methods for Examples 1-5

### Bacterial Strains, Enzymes and Plasmids.

Bacterial strains and plasmids used in this study are listed in Table 1. S. Typhimurium strains were derived from the highly virulent strain UK-1. Bacteriophage P22HTint was used for generalized transduction. Escherichia coli and S. Typhimurium cultures were grown in LB broth or on LB agar plates at 37° C. LB agar without NaCl and with 5% sucrose was used for sacB gene-based counter-selection in allelic exchange experiments. Diaminopimelic acid (DAP) was added at the concentration of 50 μg/ml for the growth of Asd⁻ strains. For host-regulated delayed lysis vector combinations, LB was supplemented with 0.2% arabinose.

TABLE 1
Bacterial strains and plasmids used in this study

| Strain or | Genotype or Relevant | Source or |
|---|---|---|
| plasmid | Characteristics | reference |
| Strains | | |
| E. coli | | |
| TOP 10 cells | | |
| χ6212 | Φ80d lacZΔM15 deoR Δ(lacZYA-argF)- | Invitrogen |
| | U169 glnV44 λ⁻ gyrA96 recA1 endA1 | |
| | ΔasdA4 Δzhf-2::Tn10 hsdR17 (R⁻M⁺) | |
| χ7213 | thi-1 thr-1 leuB6 glnV44 fhuA21 lacY1 | MGN-614 |
| | recA1 RP4-2-Tc ::Mu[λ pir] ΔasdA4 | (3). |
| | (Δzhf-2::Tn10) | |
| S. enterica | | |
| serovar | | |
| Typhimurium | | |
| χ8633 | ΔrelA ΔspoT | MGN -4860s |
| χ8926 | ΔsifA26 | Lab |
| | | collection |
| χ9477 | ΔasdA27::TT araC P_{BAD} c2 | Lab |
| | | collection |
| χ8916 | ΔphoP233 ΔasdA16 | Lab |
| | | collection |
| χ99\tilde{3} | ΔphoP233 ΔasdA16 atrB13::MudJ | Lab |
| | | collection |
| χ11001 | ΔrelA ΔspoT ΔasdA27::TT araC P_{BAD} | This study |
| | c2 | |
| χ11017 | ΔasdA27::TT araC P_{BAD} c2ΔaraBAD23 | Lab |
| | Δ(gmd-fcl)-26 Δpmi- | collection |
| | 2426ΔrelA198::araC P_{BAD} lacI TT | |
| | ΔP_{murA25}::TT araC P_{BAD} murA | |
| χ11246 | ΔasdA27::TT araC P_{BAD} c2ΔaraBAD23 | This study |
| | Δ(gmd-fcl)-26 Δpmi- | |
| | 2426ΔrelA198::araC P_{BAD} lacITT | |
| | ΔP_{murA25}::TT araC P_{BAD} murA ΔsifA | |
| Plasmids | | |
| pCAGGS-NP | Vector containing NP gene from | Provided by |
| | A/WSN/33 | Andrew |
| | | Pekosz |
| pUC57-WSN- | Commercial vector pUC-57 containing | Synthesized |
| NP | codon optimized A/WSN/33 NP gene | by Genscript |
| pYA3681 | Lysis vector P_{trc} promoter | Kong, 2008 |
| pYA3869 | Asd⁺ vector containing Salmonella | Lab |
| | typhimurium ATG-sopE (1-80 aa) | collection |
| | pSC101 ori | |
| pYA3870 | Asd⁺ vector containing Salmonella | (2) |
| | typhimurium ATG-sopE (1-80 aa) p15A | |
| | ori | |
| pYA4631 | C-terminus (CT) (from nucleotide 772- | This study |
| | 1505) of NP gene cloned in Zero blunt | |
| | cloning vector (Invitrogen) | |
| pYA4632 | Asd vector carrying 3 x Flag | This study |
| | downstream of Cla1 site; p15A ori | |
| pYA4629 | SopE-CT-NP 3 x Flag tag; p15A ori | This study |
| pYA4630 | SopE-CT-NP 3 x Flag tag; pSC101 ori | This study |
| pYA4247 | pYA3870 carrying ESAT-6 and CFP- | Lab |
| | 10 + 3 x Flag tag; p15A ori | collection |
| pYA4700 | SopE-NP-Flag tag; p15A ori | This study |
| pYA4701 | SopE-NP-Flag tag; pSC101 ori | This study |
| pYA4699 | SopE-NP epitope (147-155)- | This study |
| | 3 x Flag tag in zero blunt cloning | |
| | vector (Invitrogen) | |
| pYA4702 | P_{trc} promoter pBR ori NP gene | This study |
| pYA4858 | Lysis vector pBR ori codon optimized | This study |
| | NP gene | |
| pYA4651 | Lysis vector P_{trc} promoter pBR ori ply | Lab |
| | gene | collection |
| Suicide Vectors | | |
| pYA4138 | Suicide vector for ΔasdA27::TT araC | Lab |
| | P_{BAD} c2 | collection |
| pYA3716 | Suicide vector for ΔsifA26 | Lab |
| | | collection |

1. Kong, W., S. Y. Wanda, X. Zhang, W. Bollen, S. A. Tinge, K. L. Roland, and R. Curtiss, 3rd. Regulated programmed lysis of recombinant Salmonella in host tissues to release protective antigens and confer biological containment. Proc Natl Acad Sci USA, 2008, pp. 9361-9366, Vol. 105.
(2) Konjufca, V., S. Y. Wanda, M. C. Jenkins, and R. Curtiss, 3rd. A recombinant attenuated Salmonella enterica serovar Typhimurium vaccine encoding Eimeria acervulina antigen offers protection against E. acervulina challenge. Infect Immun, 2006, pp. 6785-6796, Vol. 74.
(3) Roland, K., R. Curtiss III and D. Sizemore. Constrcution and evaluation of a delta cya delta crp Salmonella typhimurium strain expressing avian pathogenic Escherichia coli O78 LPS as a vaccine to prevent airsacculitis in chickens. Avian diseases, 1999, pp. 429-441, Vol. 43.

### Strain Construction and Characterization.

The phage lysate for ΔasdA27::TT araC P_{BAD} c2 was prepared from strain χ9477 by conjugating it with E. coli χ7213(pYA4138) by the standard method. The mutation ΔasdA27::TT araC P_{BAD} c2 was introduced by transduction into the strain χ8633 resulting in strain χ11001. Colonies were screened for chloramphenicol sensitivity and DAP dependency and verified by PCR.

The ΔsifA26 mutation is a defined in-frame deletion of the sifA gene. It was introduced into strain χ11017 by phage P22 transduction from (χ8926::pYA3716) to generate strain χ11246. The presence of the mutation was verified by PCR. The presence of the ΔasdA27::TT araC P_{BAD} c2 mutation in Salmonella was confirmed by its dependence on DAP for growth. The presence of the ΔP_{murA25}::TT araC P_{BAD} murA mutation (Table 1) was verified by its dependence on arabinose for growth. LPS profiles were examined as previously described.

The lysis phenotype of the bacterial strains was confirmed by diluting overnight cultures 10⁻³ and 10⁻⁴ and plating 100 μl samples on LB plates with or without 0.2% arabinose followed by incubation at 37° C. Strains displaying regulated delayed lysis were grown on LB agar containing arabinose only, depicting complete dependence on the presence of arabinose for survival.

### General DNA Procedures.

DNA manipulations were carried out as described by Sambrook et. al. [32]. Transformations of E. coli and S. Typhimurium were done by electroporation (Bio-Rad, Hercules, Calif.). Transformants containing Asd⁺ plasmids were selected on LB agar plates without DAP.

### Plasmid Stability.

Plasmid stability was determined as described before [24]. RASV strains harboring the plasmids pYA4702 or pYA4858 were grown overnight in 3 ml cultures supplemented with 50 μg/ml of DAP and 0.2% arabinose. Next day, fresh LB supplemented with DAP and arabinose was inoculated with a 1:1000 dilution of overnight culture and grown statically at 37° C. overnight (about 14 hours). To estimate the proportions of bacterial cells retaining the Asd⁺ plasmid, cultures were serially diluted and 10⁻⁵ and 10⁻⁶ were plated on LB plates supplemented with DAP and arabinose and grown overnight at 37° C. Next day, 100 colonies from these plates were picked and patched onto LB supplemented with arabinose and LB supplemented with DAP and arabinose. Percentage of clones retaining the plasmids was determined by counting the colonies. Concurrently, colonies from each day's plating were grown in fresh LB broth supplemented with DAP and arabinose and the process repeated for 5 consecutive days (50 generations). At the end, a representative number of colonies were screened to possess Asd⁺ plasmids of the correct size and copy number and to encode synthesis of a protective antigen of the correct size.

### Peptide.

Synthetic peptide NP₁₄₇₋₁₅₅ (TYQRTRALV) was obtained from Biosynthesis Inc. (Lewisville, Tex.). It was dissolved in water according to the manufacturer's instruction, aliquoted, and stored at −20° C. until used.

### Codon Optimization of NP Gene.

The sequence of the nucleoprotein (NP) gene of influenza virus strain A/WSN/33 (NCBI, accession number EU330203) was codon optimized for maximal expression in Salmonella. The gene sequence was commercially codon-optimized and cloned in pUC-57 to yield pUC-57-NP-WSN by Genscript (Piscataway, N.J.). The replaced codons are depicted in Table 2. The average G+C content was changed from 46.66 for the non-optimized gene to 54.59 after codon optimization. The stem-loop structures, which impact ribosomal binding and stability of mRNA were disrupted. The codon usage bias for E. coli was increased from a codon adaptation index of 0.57 to 0.98.

TABLE 2
Codon optimized versus original nucleotide sequence and
amino acids for A/WSN/33 NP gene.

| | | |
|---|---|---|
| | | 45 |
| Opt | ATG GCG ACC AAA GGC ACC AAA CGT AGC TAT GAA CAG ATG GAA ACC | |
| Ori | ATG GCG ACC AAA GGC ACC AAA CGA TCT TAC GAA CAG ATG GAG ACT | |
| | M A T K G T K R S Y E Q M E T | |
| | *** *** *** *** *** *** *** ** ** *** *** *** ** ** | |
| | | 90 |
| Opt | GAT GGC GAA CGT CAG AAC GCG ACC GAA ATT CGT GCG AGC GTG GGC | |
| Ori | GAT GGA GAA CGC CAG AAT GCC ACT GAA ATC AGA GCA TCT GTC GGA | |
| | D G E R Q N A T E I R A S V G | |
| | *** ** *** ** *** ** ** ** *** ** * ** ** ** | |
| | | 135 |
| Opt | AAA ATG ATT GAT GGC ATT GGC CGT TTT TAT ATT CAG ATG TGC ACC | |
| Ori | AAA ATG ATT GAT GGA ATT GGA CGA TTC TAC ATC CAA ATG TGC ACC | |
| | K M I D G I G R F Y I Q M C T | |
| | *** *** *** *** ** *** ** ** ** ** ** ** *** *** *** | |
| | | 180 |
| Opt | GAA CTG AAA CTG AGC GAT TAT GAA GGC CGT CTG ATT CAG AAC AGC | |
| Ori | GAA CTT AAA CTC AGT GAT TAT GAG GGA CGG CTG ATT CAG AAC AGC | |
| | E L K L S D Y E G R L I Q N S | |
| | *** ** *** ** ** *** *** ** ** ** *** *** *** *** *** | |
| | | 225 |
| Opt | CTG ACC ATT GAA CGT ATG GTG CTG AGC GCG TTT GAT GAA CGT CGT | |
| Ori | TTA ACA ATA GAG AGA ATG GTG CTC TCT GCT TTT GAC GAG AGG AGG | |
| | L T I E R M V L S A F D E R R | |
| | * ** ** ** * *** *** ** ** * ** ** ** * * | |
| | | 270 |
| Opt | AAC AAA TAT CTG GAA GAA CAT CCG AGC GCG GGC AAA GAT CCA AAG | |
| Ori | AAT AAA TAT CTA GAA GAA CAT CCC AGT GCG GGG AAA GAT CCT AAG | |
| | N K Y L E E H P S A G K D P K | |
| | ** *** *** ** *** *** *** ** ** *** ** *** *** ** *** | |
| | | 315 |
| Opt | AAA ACC GGC GGC CCG ATT TAT CGT CGT GTG GAT GGC AAA TGG CGT | |
| Ori | AAA ACT GGA GGA CCT ATA TAC AGG AGA GTA GAT GGA AAG TGG AGG | |
| | K T G G P I Y R R V D G K W R | |
| | *** ** ** ** ** ** ** * * ** *** ** ** *** * | |
| | | 360 |
| Opt | CGT GAA CTG ATT CTG TAT GAT AAA GAA GAA ATT CGT CGT ATT TGG | |
| Ori | AGA GAA CTC ATC CTT TAT GAC AAA GAA GAA ATA AGA CGA ATC TGG | |
| | R E L I L Y D K E E I R R I W | |
| | * *** ** ** ** *** ** *** *** *** ** * ** ** *** | |
| | | 405 |
| Opt | CGT CAG GCG AAC AAC GGC GAT GAT GCG ACC GCG GGC CTG ACC CAC | |
| Ori | CGC CAA GCT AAT AAT GGT GAC GAT GCA ACG GCT GGT CTG ACT CAC | |
| | R Q A N N G D D A T A G L T H | |
| | ** ** ** ** ** ** ** *** ** ** ** ** *** ** *** | |
| | | 450 |
| Opt | ATG ATG ATT TGG CAT AGC AAC CTG AAC GAT GCG ACC TAT CAG CGT | |
| Ori | ATG ATG ATC TGG CAC TCC AAT TTG AAT GAT GCA ACT TAC CAG AGG | |
| | M M I W H S N L N D A T Y Q R | |
| | *** *** ** *** ** * ** ** ** *** ** ** ** *** * | |
| | | 495 |
| Opt | ACC CGT GCG CTG GTG CGT ACC GGC ATG GAC CCA CGT ATG TGC AGC | |
| Ori | ACA AGA GCT CTT GTT CGC ACA GGA ATG GAT CCC AGG ATG TGC TCA | |
| | T R A L V R T G M D P R M C S | |
| | ** * ** ** ** ** ** ** *** ** ** * *** *** | |
| | | 540 |
| Opt | CTG ATG CAG GGC AGC ACC CTG CCG CGT CGT AGC GGT GCA GCA GGT | |
| Ori | CTG ATG CAG GGT TCA ACC CTC CCT AGG AGG TCT GGG GCC GCA GGT | |
| | L M Q G S T L P R R S G A A G | |
| | *** *** *** ** *** ** ** * * ** ** *** *** | |
| | | 585 |
| Opt | GCA GCA GTG AAA GGC GTG GGT ACG ATG GTG ATG GAA CTG ATT CGT | |
| Ori | GCT GCA GTC AAA GGA GTT GGA ACA ATG GTG ATG GAA TTG ATC AGA | |
| | A A V K G V G T M V M E L I R | |
| | ** *** ** *** ** ** ** ** *** *** *** *** ** ** * | |
| | | 630 |
| Opt | ATG ATT AAA CGT GGC ATT AAC GAT CGT AAC TTT TGG CGT GGC GAA | |
| Ori | ATG ATC AAA CGT GGG ATC AAT GAT CGG AAC TTC TGG AGG GGT GAG | |
| | M I K R G I N D R N F W R G E | |
| | *** ** *** *** ** ** ** *** ** *** ** *** * ** ** | |
| | | 675 |
| Opt | AAC GGC CGT CGT ACC CGT ATT GCG TAT GAA CGT ATG TGC AAC ATT | |
| Ori | AAT GGA CGG AGA ACA AGG ATT GCT TAT GAA AGA ATG TGC AAC ATT | |
| | N G R R T R I A Y E R M C N I | |
| | ** ** ** * ** * *** ** *** *** * *** *** *** *** | |
| | | 720 |
| Opt | CTG AAA GGC AAA TTT CAG ACC GCG GCG CAG CGT ACG ATG GTG GAT | |
| Ori | CTC AAA GGG AAA TTT CAA ACA GCT GCA CAA AGA ACA ATG GTG GAT | |
| | L K G K F Q T A A Q R T M V D | |
| | ** *** ** *** *** ** ** ** ** ** * ** *** *** *** | |
| | | 765 |
| Opt | CAA GTG CGT GAA AGC CGT AAC CCG GGC AAC GCG GAA TTT GAA GAC | |
| Ori | CAA GTG AGA GAG AGC CGG AAT CCA GGA AAT GCT GAG TTC GAA GAT | |
| | Q V R E S R N P G N A E F E D | |
| | *** *** * ** *** ** ** ** ** ** ** ** ** *** ** | |
| | | 810 |
| Opt | CTG ATT TTT CTG GCG CGT AGC GCG CTG ATT CTG CGT GGC AGC GTG | |
| Ori | CTC ATC TTT TTA GCA CGG TCT GCA CTC ATA TTG AGA GGG TCA GTT | |
| | L I F L A R S A L I L R G S V | |
| | ** ** *** * ** ** ** ** ** ** * ** ** | |
| | | 855 |
| Opt | GCG CAT AAA AGC TGC CTG CCG GCG TGC GTG TAT GGC AGC GCG GTG | |
| Ori | GCT CAC AAG TCC TGC CTG CCT GCC TGT GTG TAT GGA TCT GCC GTA | |
| | A H K S C L P A C V Y G S A V | |
| | ** ** ** * *** *** ** ** ** *** *** ** ** ** | |
| | | 900 |
| Opt | GCG AGC GGC TAT GAT TTT GAA CGT GAA GGC TAT AGC CTG GTG GGC | |
| Ori | GCC AGT GGA TAC GAC TTT GAA AGA GAG GGA TAC TCT CTA GTC GGA | |
| | A S G Y D F E R E G Y S L V G | |
| | ** ** ** ** ** *** *** * ** ** ** ** ** ** | |
| | | 945 |
| Opt | ATT GAT CCG TTT CGT CTG CTG CAG AAC AGC CAG GTG TAT AGC CTG | |
| Ori | ATA GAC CCT TTC AGA CTG CTT CAA AAC AGC CAA GTA TAC AGC CTA | |
| | I D P F R L L Q N S Q V Y S L | |
| | ** ** ** ** * *** ** ** *** *** ** ** ** *** ** | |
| | | 990 |
| Opt | ATT CGT CCG AAC GAA AAC CCG GCG CAT AAA AGC CAG CTG GTG TGG | |
| Ori | ATC AGA CCA AAT GAG AAT CCA GCA CAC AAG AGT CAA CTG GTG TGG | |
| | I R P N E N P A H K S Q L V W | |
| | ** * ** ** ** ** ** ** ** ** ** ** *** *** *** | |
| | | 1035 |
| Opt | ATG GCG TGC CAT AGC GCG GCG TTT GAA GAC CTG CGT GTG AGC AGC | |
| Ori | ATG GCA TGC CAT TCT GCT GCA TTT GAA GAT CTA AGA GTA TCA AGC | |
| | M A C H S A A F E D L R V S S | |
| | *** ** *** *** ** ** *** *** ** ** * ** *** | |
| | | 1080 |
| Opt | TTT ATT CGT GGC ACC AAA GTG GTG CCG CGT GGC AAA CTG AGC ACC | |
| Ori | TTC ATC AGA GGG ACG AAA GTG GTC CCA AGA GGG AAG CTT TCC ACT | |
| | F I R G T K V V P R G K L S T | |
| | ** ** * ** ** *** *** ** ** * ** ** ** * ** | |
| | | 1125 |
| Opt | CGT GGC GTG CAG ATT GCG AGC AAC GAA AAC ATG GAA ACG ATG GAA | |
| Ori | AGA GGA GTT CAA ATT GCT TCC AAT GAA AAC ATG GAG ACT ATG GAA | |
| | R G V Q I A S N E N M E T M E | |
| | * ** ** ** *** ** * ** *** *** *** ** ** *** *** | |
| | | 1170 |
| Opt | AGC AGC ACC CTG GAA CTG CGT AGC CGT TAT TGG GCG ATT CGT ACC | |
| Ori | TCA AGT ACC CTT GAA CTG AGA AGC AGA TAC TGG GCC ATA AGG ACC | |
| | S S T L E L R S R Y W A I R T | |
| | ** *** ** *** *** * *** * ** *** ** ** * *** | |
| | | 1215 |
| Opt | CGT AGC GGC GGC AAC ACC AAC CAG CAG CGT GCG AGC AGC GGC CAG | |
| Ori | AGA AGT GGA GGG AAC ACC AAT CAA CAG AGG GCT TCC TCG GGC CAA | |
| | R S G G N T N Q Q R A S S G Q | |
| | * ** ** ** *** *** ** ** *** * ** * *** ** | |
| | | 1260 |
| Opt | ATT AGC ATT CAG CCG ACC TTT AGC GTG CAG CGT AAC CTG CCG TTT | |
| Ori | ATC AGC ATA CAA CCT ACG TTC TCA GTA CAG AGA AAT CTC CCT TTT | |
| | I S I Q P T F S V Q R N L P F | |
| | ** *** ** ** ** ** ** ** *** * ** ** ** *** | |
| | | 1305 |
| Opt | GAT CGT CCG ACC ATT ATG GCG GCG TTT ACC GGC AAC ACC GAA GGC | |
| Ori | GAC AGA CCA ACC ATT ATG GCA GCA TTC ACT GGG AAT ACA GAG GGG | |
| | D R P T I M A A F T G N T E G | |
| | ** * ** *** *** *** ** ** ** ** ** ** ** ** ** | |
| | | 1350 |
| Opt | CGT ACC AGC GAT ATG CGT ACC GAA ATT ATT CGT CTG ATG GAA AGC | |
| Ori | AGA ACA TCT GAC ATG AGA ACC GAA ATC ATA AGG CTG ATG GAA AGT | |
| | R T S D M R T E I I R L M E S | |
| | * ** ** *** * *** *** ** ** * *** *** *** ** | |
| | | 1395 |
| Opt | GCG CGT CCG GAA GAT GTG AGC TTT CAG GGC CGT GGC GTG TTT GAA | |
| Ori | GCA AGA CCA GAA GAT GTG TCT TTC CAG GGG CGG GGA GTC TTC GAG | |
| | A R P E D V S F Q G R G V F E | |
| | ** * ** *** *** *** ** *** ** ** ** ** ** ** | |
| | | 1440 |
| Opt | CTG AGC GAT GAA AAA GCG ACC AGC CCG ATT GTG CCG AGC TTT GAT | |
| Ori | CTC TCG GAC GAA AAG GCA ACG AGC CCG ATC GTG CCC TCC TTT GAC | |
| | L S D E K A T S P I V P S F D | |
| | ** ** *** ** ** ** *** *** ** *** ** * *** ** | |
| | | 1485 |
| Opt | ATG AGC AAC GAA GGC AGC TAC TTT TTC GGC GAT AAC GCG GAA GAA | |
| Ori | ATG AGT AAT GAA GGA TCT TAT TTC TTC GGA GAC AAT GCA GAG GAG | |
| | M S N E G S Y F F G D N A E E | |
| | *** ** ** *** ** ** ** *** ** ** ** ** ** ** | |
| Opt | TAT GAT AAC TAA | |
| Ori | TAC GAC AAT TAA | |
| | Y D N | |
| | *** ** ** *** | |

Opt = codon optimized sequence; Ori = Non-codon optimized original sequence.

### Vector Construction.

The primer pairs used in this study are listed in Table 3. Vent DNA polymerase was used for the PCR reaction with dNTPs (invitrogen).

TABLE 3
Primer pair sequences.

| Primers | Sequence 5′ - 3′ |
|---|---|
| TTFP-3 | CGGAATTCTTAGCACGGTCTGCACTCAT |
| TTRP-3 | CCCGGGAATTGCTTAATTGTCGTACTCC |
| TTFCLa-5 | GTCGAATGCTGCGCCAGTTGGCGTAG |
| TTFLag | CCCCCATCGATGGACGGATCCCCGGGAATTGCGATGAG |
| R-6 | ATCTTCGAACT |
| NP-epi | GCAGTGTTGACAAATGAATTCTCCAATTTGAATGATGC |
| 147 | AACTTACCAGAGGACAAGAGCTCTTGTTCGCACAGGAA |
| | TGGATCCCAGGATGTGCATCGATGAC |
| NP-Epi-F2 | CCGGAATTCTCCAATTTGAATGAT |
| NP-Epi-R3 | TCCCCCCGGGAATTGCTTACTATTTATCGTCG. |
| RDLF-3 | CATGCCATGGCGACCAAAGGCACCAAACGA |
| RDLP-2 | TCCCCCCCGGGTTACTATTTATCGTCGTCATCTTTGTA |
| | GTCGATATCATGATCTTTATAATCACCGTCATGGTCTT |
| | TGTAGTCATTGTCGTACTCCTCTGCATTGTCTCCGAA |
| RDLF-5 | ATGCCATGGCGATGGCGACCA |
| RDLRP-7 | CTATTACCATGGGTTATCATATTCTTCCGCG |
| codNP | CATGCCATGGCTAGTGGTGGTGGTGGTGGTGGTTATCA |
| hisR1 | TATTCTTCCGCGTTA |
| P_{trc}-F | ATTCTGAAATGAGCTGTT |
| P_{trc}-R | TCTCATCCGCCAAAACAGCC |

### Type III Secretion System (T3SS) Vectors.

Asd vectors containing the promoter region and nucleotide sequence encoding the terminal secretion and translocation domain (1-80 aa) of S. Typhimurium ATG-sopE with pSC101 ori, pYA3869 and with p15 ori, pYA3870 have been described before [33]. The C-terminus region (CT) of NP (from nucleotides 772-1505) or an NP (147-155) epitope was inserted into these two vectors.

The CT of the NP gene was amplified from pCAGGS-NP (kindly provided by Dr. Andrew Pekosz, Washington University, St. Louis) using the primers TTFP-3 and TTRP-3 and cloned into Zero blunt cloning kit (Invitrogen) to yield pYA4631. Plasmid pYA4247 carrying SopE-ESAT-6 and CFP-10 was digested with Cla1, to get rid of these sequences and re-ligated to yield pYA4632, which is similar to pYA3870 except that it contains a 3×FLAG tag on the C-terminus end. The NP-CT fragment from pYA4631 was digested with EcoR1 and Xma1 and ligated into pYA3870 to yield pYA4629. The SopE-CT-NP-Flag fragment was amplified from pYA4629 using primers TTFCla-5 and TTFlagR-6, digested with Cla1 and cloned into pYA3869 yielding pYA4630.

To fuse SopE with the NP epitope, plasmid pYA4247 was amplified using the primers TTFCla-5 and NP-epi147 and the PCR product was cloned into Zeroblunt PCR cloning Kit (Invitrogen) yielding pYA4699. The SopE-NP epitope fusion was digested with Cla1 and subcloned into pYA4632 to yield pYA4700. pYA4700 was amplified with the primers NP-Epi-F2 and NP-Epi-R3 and the PCR product was digested with EcoR1 and Xma1 enzymes and cloned into pYA3869 to yield pYA4701. The plasmids were transformed into χ8916 (ΔphoP233 ΔasdA16) and χ11001 (ΔrelA ΔspoT ΔasdA27::TT araC P_{BAD} c2) for in vitro secretion analysis and vaccination experiments.

### Regulated Lysis Vectors.

The NP gene was amplified from plasmid pCGGAS-NP (kindly provided by Dr. Andrew Pekosz) by PCR using the primer pair RDLF-3 and RDLP-2. The PCR product was digested using NcoI and XmaI sites and cloned into plasmid pYA3681 yielding pYA4702. The codon-optimized NP gene from pUC-57-WSN-NP was amplified using the primer pair RDLF-5 and RDLRP-7. The PCR product was digested with NcoI and cloned in pYA3681 yielding pYA4858. The correct orientation of the NP gene was confirmed by restriction digestion with PstI and by sequencing. All derivatives of pYA3681 were sequenced by the primer set P_{trc}-F and P_{trc}-R, respectively. Negative control vector pYA4651 encoding the ply gene from S. pneumonia cloned in pYA3681 was constructed by Wei Xin. All vectors were transferred to appropriate S. Typhimurium strains by electroporation. All DNA constructs were confirmed by sequencing at the core facility at Arizona State University, using ABI Prism fluorescent BigDye terminators.

Secretion of sopE-NP into Culture Supernatant.

Secretion of SopE-CT-NP or SopE-NP epitope was analyzed according to the procedure described before [24]. Briefly, RASV strain cultures were grown in LB containing 300 mM NaCl with gentle aeration to an O.D₆₀₀ of 0.6. Cells were centrifuged at 6000×g for 15 min, filtered through a 0.45 μm filter, precipitated overnight with 10% trichloroacetic acid (TCA) and pelleted by centrifugation at 13000×g for 15 min. The pellets were washed with ice-cold acetone, air-dried, resuspended in SDS-PAGE sample buffer and analyzed by SDS-PAGE and western blots. To make sure that antigen is secreted via the T3SS and not by lysis of cells, the supernatant and culture were analyzed simultaneously for RpoD^{σ70} as an indicator of membrane leakage.

### SDS-PAGE and Immunoblots.

To evaluate NP protein synthesis from plasmids in E. coli and S. Typhimurium strains, bacterial cells were grown overnight at 37° C. in LB containing 0.2% arabinose. Aliquots (1 ml) were taken, centrifuged at low speed, and resuspended in 2×SDS-PAGE loading buffer and boiled for 10 min. The samples were centrifuged for 10 min, diluted 1:10 in 2× sample loading buffer and 10 μl was loaded onto 12.5% SDS-PAGE gels for separation by electrophoresis as previously described [44]. Proteins were transferred onto nitrocellulose membranes and blocked with 5% skim milk for 1 h at room temperature. Membranes were rinsed with PBS-0.05% Tween 20 (T20) three times. For analyzing NP synthesis blots were incubated with rabbit polyclonal anti-influenza A NP antibody (Abcam) for 1 h with constant shaking. After washing with PBS-T20, the membranes were incubated with goat anti-rabbit IgG alkaline phosphatase conjugate (Sigma) for 1 h and developed with nitroblue tetrazolium-5-bromo-4-chloro-3-indolyphosphate (BCIP) (Sigma). Membranes were washed with water and airdried.

For T3SS analysis the blots were probed with anti-Flag antibody (Sigma) and Anti-β galactosidase antibody (Abcam) as a marker for secretion and secondary antibodies were anti-mouse IgG and anti-rabbit IgG, respectively (Sigma).

### Virus Strain, Propagation, Purification and Titration.

rWSN virus was provided by Dr. Andrew Pekosz (Johns Hopkins University, Baltimore, Md.). It is a mouse-adapted strain created by reverse genetics and is lethal to mice in doses above 10³ TCID₅₀. The virus was propagated and titrated in Madin-Darby canine kidney cells cultured in RPMI-1640 (Gibco) containing 2 μg/ml acetyl-trypsin (Sigma). The virus was passed through a 30% (w/v) sucrose cushion at 11,620×g for 3 h in a Surespin Sorvall 630 rotor using a WK ultra 90 centrifuge (Thermo Electron Corp.). The resulting pellet was resuspended in phosphate buffered saline (PBS) pH 7.2 and centrifuged at 11,620×g for 1 h. The viral pellet was finally dissolved in 500 μl of PBS and kept frozen at −80° C. until used.

### Immunization of Mice.

All animal experiments were done in BSL-2 level containment in our animal facilities at The Biodesign Institute, Arizona State University, according to approved ASU IACUC protocols. Five-week old female BALB/c mice were purchased from Charles River Laboratories (Wilmington, Mass.) and were allowed to acclimate for 1 week before immunization. Each group of mice was deprived of food and water for 4 h prior to oral immunization. Recombinant S. Typhimurium strains were individually grown in LB broth with 0.2% arabinose and 0.2% mannose to an OD₆₀₀ of 0.85. The cultures were centrifuged at 4,000×g for 15 min at room temperature and suspended in buffered saline containing 0.01% gelatin (BSG) to a final concentration of 5×10⁹ CFU/ml. Bacteria were titrated on LB agar supplemented with arabinose. Mice were immunized by the peroral (PO) route with 20 μl (1×10⁹ CFU), intranasally (IN) 10 μl (1×10⁷ CFU) or by the intraperitoneal (IP) route with 100 μl (1×10⁵ CFU). Food and water were returned to orally immunized mice 30 min after vaccine administration. Vectors without any expressed antigen gene (pYA3681) or that expressed the ply gene from S. pneumonia (pYA4651) and BSG immunized mice were used as negative controls in the following experiments.

### Regulated Lysis Strains.

Vector without any expressed antigen pYA3681 or pYA4651 expressing ply antigen from Streptococcus pneumoniae and BSG vaccinated mice were used as negative controls in the following experiments. Blood was drawn by cheek pouch bleeding, allowed to clot for 30 min in a 37° C. incubator and left overnight at 4° C. Sera were collected after centrifugation at 10,000×g for 15 minutes and stored at −20° C. till used and tested by ELISA for the presence or absence of antibodies against NP or LPS as described below. For assaying cellular immune responses, spleens were collected aseptically, pooled and processed for ELISPOT and Intracellular cytokine staining (ICS) as described below.

### Animal Experiment 1.

To evaluate the effect of the sifA deletion (χ11246) on the immunogenicity and protective immunity conferred by RASV strains encoding the codon-optimized NP (pYA4858) of influenza virus, BALB/c mice (n=8) were orally immunized with parent χ11017(pYA4858) (SifA⁺), mutant χ11246(pYA4858) (SifA⁻), vector controls χ11017(pYA3681) (SifA⁺) and χ11246(pYA3681) (SifA⁻) or with BSG at week zero and boosted three times at weeks 1, 4 and 7 post primary immunization (PPI). Blood collected at weeks 3 and 6 PPI by cheek pouch bleeding was monitored for the presence of antibodies against NP or S. Typhimurium LPS by ELISA. For assaying antigen specific IFN-γ secreting T cells, spleens were aseptically collected at week 8 PPI from 2-3 mice, pooled and processed for ELISPOT. The remaining mice (n=5) in each group were challenged with rWSN (100 LD₅₀) at week 8 PPI (14 weeks of age) and observed for morbidity and mortality for an additional 3 weeks.

### Animal Experiment 2.

The groups of mice (n=8) were immunized orally with strains encoding codon-optimized NP χ11246(pYA4858) (SifA⁻), an irrelevant antigen (Ply) from χ11246(pYA4651) or BSG at week zero and boosted twice at week 1 and 4 PPI. Spleens and blood were harvested from 3 mice from each group, 4 days after the final boost and ELISPOTs and ELISA were performed to detect antigen-specific T cells and NP and LPS specific antibodies. The remaining mice in each group were challenged with rWSN (100 LD₅₀) at week 5 PPI (at 10 weeks of age) and observed for morbidity and mortality for 3 additional weeks.

### Animal Experiment 3.

To determine the immunogenicity and protective efficacy of using the SifA⁻ strain when administered via different routes, mice were immunized via PO, IN or IP routes with RASV χ11246(pYA4858) (NP⁺ SifA⁻) and χ11246(pYA4651) (Ply⁺ SifA⁻) as a negative control, at week 0 and boosted thrice at weeks 1, 4 and 7 PPI. Spleens were harvested from 3 mice four days after the final immunization and analyzed for production of antigen-specific IFN-γ secreting cells by ELISPOT and for NP₁₄₇₋₁₅₅ specific proliferation. The remaining mice in each group were challenged with rWSN (100 LD₅₀) at week 8 PPI (14 weeks of age) and observed for morbidity and mortality for an additional 3 weeks.

### Virus Challenge.

For virus challenge, mice were anaesthetized with 0.05 ml/20 g body weight of a ketamine cocktail (21.0 mg ketamine, 2.4 mg xylazine, and 0.3 mg acepromazine) administered intraperitoneally. Sedated mice were intranasally (IN) infected with a 100 LD₅₀ (1×10⁵ TCID₅₀) of rWSN in a total volume of 30 μl, 15 μl per nostril for all experiments. Groups of mice were IN infected with 30 ul of the serially diluted purified rWSN virus from 1×10⁷-1×10² TCID₅₀ at 8 weeks of age and the LD₅₀ determined by the method of Reed and Muench. To rule out any age dependent variation in LD₅₀ doses, similar experiments were performed with mice at 10 and 14 weeks of age. No difference was observed in terms of virus-associated morbidity and mortality in mice at 8, 10 or 14 weeks of age. An aliquot of the virus used for challenge was back-titrated on MDCK cells to ascertain the exact dose given to mice. The challenged mice were inspected daily for signs of infection such as ruffled fur, hunched posture, and weighed on alternate days till 21 days to monitor the progression of infection. Percent weight loss was calculated for individual mice in each group by comparing their daily weight to their pre-challenge weight. Mice that succumbed to infection or had to be euthanized were promptly removed.

### ELISA.

IgG responses against NP or LPS in sera were determined by ELISA [50]. Briefly, 96-well flat-bottom polystyrene microtiter plates (Dynatech Laboratories Inc., Chantilly, Va.) were coated with 2 μg/ml of purified NP protein (kindly provided by Dr. Troy Randall, (Trudeau Institute, Saranac Lake, N.Y.)) or LPS (Sigma) suspended in carbonate coating buffer (pH=9.5) and incubated at 4° C. overnight. Free binding sites were blocked with phosphate buffered saline (PBS)-0.05% T20 containing 3% bovine serum albumin (BSA) for 2 h at room temperature. Sera were serially two-fold diluted in PBS/3% BSA and 100 μl was incubated in duplicate wells for 1 h at room temperature. Plates were washed thrice with PBS-T20 and incubated for 1 h with a 1:1000 dilution of either biotinylated goat anti-mouse IgG or IgG1 or IgG2a (Southern Biotechnology Inc., Birmingham, Ala.). After washing as above, the plates were incubated for 1 h with streptavidin-alkaline phosphatase conjugate (Southern Biotechnology Inc., Birmingham, Ala.) and developed by incubating with p-nitrophenyl phosphate (Sigma) for 30 min and read by an automated ELISA plate reader (SpectraMax, Molecular Devices, Sunnydale, Calif.) at 405 nm. Endpoint titers were expressed as the reciprocal log 2 value of the last positive sample dilution. Absorbance two times higher than pre-immune serum, used as baseline values, were considered positive.

### IFN-γ ELISPOTS.

At week 5 or 8 spleens from 2-3 vaccinated mice were aseptically collected from each group and pooled within a group. Enzyme-linked immunospot (ELISPOT) assays were performed as described before [38]. Briefly, polyvinylidene difluoride membrane plates (Millipore, Bedford, Mass.) coated with 100 μl with 5 μg/ml of anti-gamma interferon (IFN-γ) monoclonal antibodies (MAb) (BD Pharmingen, San Diego, Calif.) in PBS were held overnight at 4° C. The wells were washed with PBS and blocked with RPMI medium with 10% fetal calf serum (FCS) for 2 hours. Splenocytes in 50 μl (1,000,000 per well) of RPMI 1640 supplemented with 10% FCS, 2 mM L-glutamine, 100 IU/ml penicillin and streptomycin, and 1% HEPES, with or without peptide NP (147-155) were added per well and incubated in the plates overnight in 5% CO₂ at 37° C. Concanavalin A at 2 μg/ml was used as a positive control. The next day, the cell suspensions were discarded and the plates washed with PBS. Biotinylated anti-IFN-γ MAb (BD Pharmingen) at 0.5 μg/ml in PBS with 1% FCS was added and incubated at room temperature for 2 h. After washing with PBS, 100 μl/well of avidin peroxidase diluted 1:1,000 (vol/vol) in PBS-Tween 20 containing 1% FCS was added and followed by incubation for 1 h at room temperature. 3-Amino-9-ethylcarbazole substrate (Vector Laboratories, Burlingame, Calif.) was prepared according to manufacturer's specifications, and 100 μl of substrate was added per well. Spots were developed for 15 min at room temperature. Plates were dried and analyzed by using an automated CTL ELISPOT reader system (Cellular Technology LTD, Cleveland, Ohio).

### Cell Proliferation.

Lymphocyte proliferation assays were performed to assess influenza peptide specific (NP₁₄₇₋₁₅₅) cell-mediated responses. Single-cell suspensions prepared from spleens were plated at a concentration of 5×10⁵ cells/well and stimulated with the NP₁₄₇₋₁₅₅ peptide TYQRTRALV (20 μg/ml) for 7 days. Vision blue Dye™ from the fluorescence cell viability assay kit (Biovision, Mountain View, Calif.) was added according to the manufacturer's instructions and plates were read at excitation 530 nm and emission 590 nm.

### Cell Preparations and Intracellular Cytokine Assay.

Single-cell suspensions were prepared from spleens, washed twice, and filtered through a fine Nitex membrane. The samples were then cultured in cell medium (RPMI-1640 supplemented with 10% FCS, 2 mM L-glutamine, 100 IU/ml penicillin and streptomycin, and 1% HEPES). IFN-γ secreting CD8⁺ cells were detected using the manufacturer's protocol (eBioscience, San Diego, Calif.) [39]. Briefly, cultures were stimulated for 12 h with NP peptide (147-155) TYQRTRALV or NP antigen (5 μg/ml). Monensin was added to final concentration of 2 μM 2 h before the end of the incubation. The cells were washed with washing buffer (3% FBS in PBS) and blocked with purified anti-mouse CD16/32 to block nonspecific staining via FcRII/III. Cells were stained with Phycoerythrin-Cy5 (PE-cy5) conjugated anti-mouse CD8 antibody, fixed, permeabilized and stained with anti-mouse IFN-γ antibody conjugated with phycoerythrin (PE) (eBioscience, San Diego, Calif.). All analyses were done on a FACS500 (BD Biosciences) using CXP software for data analysis.

### Statistical Analysis.

Differences in antibody titers between groups, cell proliferation and quantitative difference in numbers of IFN-γ secreting cells between the groups were determined using analysis of variance (ANOVA) and statistically different means were further analyzed using Bonferroni's test or by Tukey's method. Survival analysis was analyzed using the log rank test (GraphPad Prism; GraphPad Software).

### Example 1

### Type Three Secretion System (T3SS) Analysis

The plasmids carrying SopE fused to the C-terminus NP did not secrete significant protein through the T3SS. The pSC101 on plasmid secreted the SopE-NP (147-158) better than the plasmid specifying the same fusion on a medium copy number plasmid (p15A ori). By changing the start codon from ATG to GTG in pYA4762 the secretion diminished (FIG. 1). T-cell responses were not detected in such RASV strains delivered orally or intranasally (data not shown). All of these constructs did not induce protection to mice against viral challenge (data not shown). These results indicated that the T3SS is not always able to deliver foreign protective antigens to the cytosol and is therefore sometimes an inferior means to deliver antigens to induce a T cell dependent immune response. Another means to augment induction of cellular immunity would be to deliver increased amounts of the NP antigen by cell lysis rather than by the T3SS, which did not work for delivery of the NP antigen.

### Example 2

### Regulated Lysis System

Since T3SS seems limited in its ability to secrete the C-terminus of the NP protein, the regulated delayed lysis system was used as an alternative approach to deliver NP. The complete NP gene carrying a 3×FLAG tag at the C-terminus of the gene was cloned into pYA3681 yielding pYA4702 and delivered by strain χ11001, a non-lysis strain believed to be able to induce a strong cellular immune response. The RASV χ11001 delivering pYA4702 showed lymphocyte cell proliferation when stimulated with the peptide (FIG. 2). However, these constructs did not induce protection to mice against viral challenge (data not shown). One potential reason for these results would be non-delivery of sufficient protective NP antigen to elicit a cellular immune response. For improving the expression of NP, the codons were optimized for maximal expression in Salmonella. The pYA4858 vector with the codon optimized sequence synthesized higher amounts of protein than the pYA4702 vector with the non-codon optimized NP sequence in χ11017 as detected by SDS-PAGE followed by western blots using rabbit polyclonal anti-NP antisera (FIG. 3).

### Example 3

### Results of Animal Experiment 1

Mice orally immunized with RASV χ11017(pYA4858) (SifA⁺) and χ11246(pYA4858) (SifA⁻), strains that both exhibit a regulated delayed lysis in vivo phenotype, both induced significantly (P<0.001) higher antibody titers against influenza NP and against Salmonella LPS as compared to BSG (FIG. 4A). The antibody titers elicited against NP by immunization with either χ11017(pYA4858) (SifA⁺) or χ11246(pYA4858) (SifA⁻) were similar indicating that both RASV strains were equally immunogenic. The antibody titers elicited against LPS by χ11017(pYA4858) (SifA⁺), χ11246(pYA4858) (SifA⁻) and the vector controls were similar indicating that all vectors invaded the host cells and colonized lymphoid tissues equally well. The antibody responses against influenza NP were skewed towards IgG2a, a typical Th1-type response elicited by RASV (FIG. 4B).

Mice infected with the rWSN influenza strain showed ruffled fur, hunched posture, trembling and a continuous weight loss as signs of infection from the second day after challenge that progressed with time. Mice immunized with χ11246(pYA4858) (SifA⁻) recovered from influenza infection earlier as indicated by the alleviation of symptoms by 6 days after challenge, than mice immunized with χ11017(pYA4858) (SifA⁺) and with vector control groups that continued to loose weight and became sicker. This is also evident by weight recovery data of mice immunized with χ11246(pYA4858) (SifA⁻) as compared to mice immunized with χ11017(pYA4858) (SifA⁺) or with vector controls χ11017(pYA3681) (SifA⁺), χ11246(pYA3681) (SifA⁻) or BSG (FIG. 5). Mice immunized with strain χ11246(pYA4858) (SifA⁻) survived whereas mice immunized with χ11017(pYA4858) (SifA⁺) and vector controls χ11017(pYA3681) and χ11246(pYA3681) or with BSG commenced dying 8 days after challenge. All mice immunized orally with χ11246(pYA4858) were protected (100%) against the 100 LD₅₀ rWSN virus challenge as compared to 25% survivors in the group immunized with χ11017(pYA4858) and 0% to 20% survivors in the groups immunized with χ11017(pYA3681) and χ11246(pYA3681) (vector controls) or BSG (FIG. 5). It is evident from these results that delivery of NP by regulated delayed lysis in the cytosol as permitted when NP was delivered by χ11246(pYA4858), which due to the ΔsifA26 mutation is able to escape the endosome to lyse in the cytosol, induces a protective immune response not achieved by other means of immunization with RASV strains without all the attributes of χ11246(pYA4858).

### Example 4

### Results of Animal Experiment 2

To determine the optimal number of booster immunizations required to protect mice from lethal virus challenge, we reduced the number of booster immunizations from 3 in the previous trial to 2 immunizations in this trial given at 1 and 4 weeks PPI. The mice were challenged with the rWSN influenza virus (100 LD₅₀) at week 5 PPI. Mice immunized with RASV χ11246(pYA4858) (SifA⁻) elicited significantly higher (P<0.001) IgG antibodies against Influenza NP as compared to the mice immunized with χ11246 (pYA4651) (SifA⁻) encoding irrelevant Ply antigen or with BSG (FIG. 6). The titers against LPS were lower in the mice immunized with χ11246(pYA4858) as compared to the vector control group probably due to attenuation of the strain resulting from over synthesis of NP. The antibody levels obtained at 5 weeks PPI were similar to the ones obtained after two immunizations at 6 weeks PPI in the previous trial (FIG. 6).

Measurement of antigen specific IFN-γ secreting T cells in Trial 2 was done by stimulating the splenocytes harvested from immunized mice in each group at 4 week PPI, 4 days after the last immunization with either purified NP protein or with the NP₁₄₇₋₁₅₅ peptide or ConA as a positive control in an ELISPOT assay. There were no influenza-specific IFN-γ secreting T cells after stimulation with either the NP protein or the NP₁₄₇₋₁₅₅ peptide (FIG. 7).

Following challenge with 100 LD₅₀ of rWSN, mice immunized with RASV χ11246(pYA4858) (NP⁺) (SifA⁻) recovered from influenza infection and commenced to regain weight whereas mice receiving either an irrelevant antigen (Ply) or BSG continued to loose weight and did not recover (FIG. 8). Mice boosted twice with χ11246(pYA4858) (NP⁺) (SifA⁻) were significantly protected against 100 LD₅₀ of rWSN of influenza virus (66% survival) as compared to 22% survival of mice in groups immunized with χ11246(pYA4651) delivering S. pneumoniae Ply as a negative control and BSG (P>0.05) (FIG. 8). These results further corroborated that delivery of antigens to the cell cytosol elicit immune responses that are more protective than when antigens with T-cell epitopes are delivered by other means and by other routes.

### Example 5

### Results of Animal Experiment 3

To investigate the efficacy of the SifA⁻ vaccine strain when administered via different routes, mice were boosted thrice (as in Trial 1) with RASV strains χ11246(pYA4858) (NP⁺) (SifA⁻) and χ11246(pYA4651) (SifA⁻) (Ply⁺) via PO, IN and IP routes. Mice immunized with RASV χ11246(pYA4858) (NP⁺) (SifA⁻) via all three routes (PO, IN and IP) elicited significantly higher (P<0.001) IgG antibodies against influenza NP and Salmonella LPS as compared to the mice orally immunized with χ11246(pYA4651) (SifA⁻) encoding an irrelevant Ply antigen or with BSG (FIG. 9). The resulting antibody responses against NP from these immunizations were of the Th1-type (IgG2a) in all cases, except that χ11246(pYA4858) (NP⁺) (SifA⁻) administered via the IP route induced a mixed IgG2a (Th1 type) and IgG1 (Th-2 type) response (FIG. 9).

A significantly higher (P<0.0001) number of influenza NP₁₄₇₋₁₅₅ peptide-specific IFN-γ secreting cells were detected in splenocytes harvested from mice at 8 weeks PPI receiving χ11246(pYA4858) (NP⁺) (SifA⁻) via the IP route than in mice immunized orally (PO) or by the intranasal (IN) route (FIG. 10).

Higher percentages of IFN-γ secreting CD8⁺ T cells were detected using ICS in groups of mice vaccinated with χ11246(pYA4858) via PO, IN and IP routes of administration as compared to χ11246(pYA4651) or BSG vaccinated groups (FIG. 11).

To assess the influenza NP₁₄₇₋₁₅₅ peptide-specific cell mediated responses, splenocytes harvested from immunized mice at 8 week PPI were stimulated with the NP₁₄₇₋₁₅₈ peptide. The degree of proliferation was measured by increase in the fluorescence of vision blue dye. Background readings from the negative control mice was subtracted from the readings from NP₁₄₇₋₁₅₈ stimulated splenocytes. The splenocytes harvested from mice immunized via the PO (P<0.05) or IP (P<0.01) route proliferated in response to NP₁₄₇₋₁₅₈ peptide as compared to splenocytes harvested from mice immunized with the negative controls (FIG. 12).

Mice infected with influenza virus showed ruffled fur, hunched posture, and trembling and weight loss as signs of infection and started dying commencing at 8 days after challenge. Mice immunized with χ11246(pYA4858) (NP⁺) (SifA⁻) via the PO and IN route recovered from infection by day 6 after challenge while those immunized by the IP route recovered earlier by 4 days after challenge as indicated by recovery from symptoms of influenza infection and weight gain (FIG. 13). Mice immunized with χ11246(pYA4858) (NP⁺) (SifA⁻) via the PO, IN and IP route of immunization were protected 80%, 100% and 100%, respectively, from the influenza virus challenge as compared to 22% in the χ11246(pYA4651) (SifA⁻) (Ply⁺) immunized group (P<0.0002) (FIG. 13).

### Discussion

Collectively, the results obtained showed that vaccination with NP does not provide sterilizing immunity against the virus. Hence we concluded that the difference in protection between SifA⁻ and SifA⁺ delayed regulated lysis vaccine strains was due to the ability of the SifA⁻ strain to deliver the NP antigen to the cytosol better than the SifA⁺ strain by programmed Salmonella lysis. Also in the absence of detectable IFN-γ and protective antibody, the protection was probably due to induction of a robust CTL response caused by releasing NP antigen in the cytosol. This newly discovered means to induce a long-lasting cellular immunity to influenza, while not likely to prevent influenza infection, will be expected to significantly reduce morbidity and mortality associated with such influenza infections. In addition, delivery of antigens to the cytosol of cells in an immunized individual by a Salmonella vaccine genetically engineered to escape the endosome and undergo lysis is a far superior means compared to use of the T3SS which is often constrained in delivery of antigens by structural attributes of antigens with T cell epitopes that decrease or even preclude their delivery through the type 3 infection needle.

### References for Examples 1-5

1. Li Y C, Norton E C, Dow W H. Influenza and pneumococcal vaccination demand responses to changes in infectious disease mortality. Health Services Research 2004; p. 905, Vol. 39 (4 Pt 1).
2. Garten R J, Davis C T, Russell C A, Shu B, Lindstrom S, Balish A, et al. Antigenic and genetic characteristics of swine-origin 2009 A (H1N1) influenza viruses circulating in humans. Science, 2009; p. 197, Vol. 325, No. 5937.
3. Smith G J, Vijaykrishna D, Bahl J, Lycett S J, Worobey M, Pybus O G, et al. Origins and evolutionary genomics of the 2009 swine-origin H1N1 influenza A epidemic. Nature; 2009; pp. 1122-1125, Vol. 459, No. 7250.
4. Bui H H, Peters B, Assarsson E, Mbawuike I, Sette A. Ab and T cell epitopes of influenza A virus, knowledge and opportunities. Proceedings of the National Academy of Sciences, 2007, p. 246, Vol. 104, No. 1.
5. Jeffrey B. Ulmer T-M F, R. Randall Deck, Arthur Friedman, Liming Guan C D, Xu Liu, Su Wang, Margaret A. Liu, John J. Donnelly M J C. Protective CD4 and CD8 T Cells against Influenza Virus Induced by Vaccination with Nucleoprotein DNA. J. Virology. 1998, pp. 5648-5653.
6. Wesley R D, Tang M, Lager K M. Protection of weaned pigs by vaccination with human adenovirus 5 recombinant viruses expressing the hemagglutinin and the nucleoprotein of H3N2 swine influenza virus. Vaccine, 2004, pp. 3427-3434, Vol. 22, Nos. 25-26.
7. Altstein A D, Gitelman A K, Smirnov Y A, Piskareva L M, Zakharova L G, Pashvykina G V, et al. Immunization with influenza A NP-expressing vaccinia virus recombinant protects mice against experimental infection with human and avian influenza viruses. Archives of Virology, 2006, pp. 921-931, Col. 151, No. 5.
8. Wraith D C, Vessey A E, Askonas B A. Purified influenza virus nucleoprotein protects mice from lethal infection. Journal of General Virology, 1987, p. 433, Vol. 68, No. 2.
9. Tite J P, Hughes-Jenkins C, O'Callaghan D, Dougan G, Russell S M, Gao X M, et al. Anti-viral immunity induced by recombinant nucleoprotein of influenza A virus. II. Protection from influenza infection and mechanism of protection. Immunology, 1990, pp. 202-207, Vol. 71, No. 2.
10. Ulmer J B, Donnelly J J, Parker S E, Rhodes G H, Feigner P L, Dwarki V J, et al. Heterologous protection against influenza by injection of DNA encoding a viral protein. Science, 1993, pp. 1745-1749, Vol. 259, No. 5102.
11. Epstein S L, Kong W P, Misplon J A, Lo C Y, Tumpey T M, Xu L, et al. Protection against multiple influenza A subtypes by vaccination with highly conserved nucleoprotein. Vaccine, 2005, pp. 5404-5410, Vol. 23, Nos. 46-47.
12. Ulmer J B, J. J. Donnelly, S. E. Parker, G. H. Rhodes, P. L. Feigner, V. J., Dwarki S H G, R. R. Deck, C. M. Dewitt, A. Friedman, L. A., Hawe K R L, D. Martinez, H. C. Perry, J. W. Shiver, D. L. Montgomery, Liu. aMA. Heterologous protection against influenza by injection of DNA encoding a viral protein. Science, 1993, pp. 1745-1749, Vol. 259.
13. Epstein S L, Stack A, Misplon J A, Lo C Y, Mostowski H, Bennink J, et al. Vaccination with DNA encoding internal proteins of influenza virus does not require CD8(+) cytotoxic T lymphocytes: either CD4(+) or CD8(+) T cells can promote survival and recovery after challenge. Int Immunol, 2000, pp. 91-101, Vol. 12, No. 1.
14. Cardenas L, Clements J D. Oral immunization using live attenuated Salmonella spp. as carriers of foreign antigens. Clinical Microbiology Reviews, 1992, p. 328, Vol. 5, No. 3.
15. Tite J P, Gao X M, Hughes-Jenkins C M, Lipscombe M, O'Callaghan D, Dougan G, et al. Anti-viral immunity induced by recombinant nucleoprotein of influenza A virus. III. Delivery of recombinant nucleoprotein to the immune system using attenuated Salmonella typhimurium as a live carrier. Immunology, 1990, p. 540, Vol. 70, No. 4.
16. Xin W, Li Y, Mo H, Roland K L, Curtiss R, 3rd. PspA family fusion proteins delivered by attenuated Salmonella enterica serovar Typhimurium extend and enhance protection against Streptococcus pneumoniae. Infect Immun, 2009, pp. 4518-4528, Vol. 77, No. 10.
17. Branger C G, Torres-Escobar A, Sun W, Perry R, Fetherston J, Roland K L, et al. Oral vaccination with LcrV from Yersinia pestis KIM delivered by live attenuated Salmonella enterica serovar Typhimurium elicits a protective immune response against challenge with Yersinia pseudotuberculosis and Yersinia enterocolitica. Vaccine, 2009, pp. 5363-5370, Vol. 27, No. 39.
18. Li Y, Wang S, Scarpellini G, Gunn B, Xin W, Wanda S Y, et al. Evaluation of new generation Salmonella enterica serovar Typhimurium vaccines with regulated delayed attenuation to induce immune responses against PspA. Proc Natl Acad Sci USA, 2009, pp. 593-598, Vol. 106, No. 2.
19. Nakayama K, S. M. Kelly, and R. Curtiss III. Construction of an Asd+ expression-cloning vector: stable maintenance and high level expression of cloned genes in a Salmonella vaccine strain. Nat Biotechnol, 1988, pp. 693-697, Vol. 6.
20. Curtiss R, III, X. Zhang, S. Y. Wanda, H. Y. Kang, V. Konjufca, Y. Li, B. Gunn, S. Wang, G. Scarpellini, and I. S. Lee. Inductions of host immune responses against Salmonella-vectored vaccines. In: K. A. Brogden N C, T. B. Stanton, Q. Zhang, L. K. Nolan, and M. J. Wannemuehler, editor. Virulence mechanisms of bacterial pathogens. 4th ed. Washington, D.C., ASM press, 2007: pp. 297-313.
21. Kong W, Wanda S Y, Zhang X, Bollen W, Tinge S A, Roland K L, et al. Regulated programmed lysis of recombinant Salmonella in host tissues to release protective antigens and confer biological containment. Proc Natl Acad Sci USA, 2008, pp. 9361-9366, Vol. 105, No. 27.
22. Srivastava I K, Liu M A. Gene vaccines. Annals of internal medicine, 2003, p. 550, Vol. 138, No. 7.
23. Galán J E. Salmonella interactions with host cells: Type III Secretion at Work. Annual review of cell and developmental biology, 2001, pp. 53-86, Vol. 17, No. 1
24. Konjufca V, Wanda S Y, Jenkins M C, Curtiss R, 3rd. A recombinant attenuated Salmonella enterica serovar Typhimurium vaccine encoding Eimeria acervulina antigen offers protection against E. acervulina challenge. Infect Immun, 2006, pp. 6785-6796, Vol. 74, No. 12.
25. Wood M W, Rosqvist R, Mullan P B, Edwards M H, Galyov E E. SopE, a secreted protein of Salmonella dublin, is translocated into the target eukaryotic cell via a sip-dependent mechanism and promotes bacterial entry. Molecular microbiology, 1996, pp. 327-338, Vol. 22, No. 2.
26. Galán J E. SnapShot: effector proteins of type III secretion systems. Cell, 2007, p. 192, Vol. 130, No. 1.
27. Rüssmann H, Shams H, Poblete F, Fu Y, Galán J E, Donis R O. Delivery of epitopes by the Salmonella type III secretion system for vaccine development. Science, 1998, p. 565, Vol. 281, No. 5376.
28. Evans D T, Chen L M, Gillis J, Lin K C, Harty B, Mazzara G P, et al. Mucosal priming of simian immunodeficiency virus-specific cytotoxic T-lymphocyte responses in rhesus macaques by the Salmonella type III secretion antigen delivery system. The Journal of Virology, 2003, p. 240, Vol. 77, No. 4.
29. Brumell J H, Goosney D L, Finlay B B. SifA, a Type III Secreted Effector of Salmonella typhimurium, Directs Salmonella-Induced Filament(Sif) Formation Along Microtubules. Traffic, 2002, pp. 407-415, Vol. 3, No. 6.
30. Beuzón C R, Méresse S, Unsworth K E, Ruiz-Albert J, Garvis S, Waterman S R, et al. Salmonella maintains the integrity of its intracellular vacuole through the action of SifA. The EMBO Journal, 2000, p. 3235, Vol. 19, No. 13.
31. Brumell J H, Tang P, Zaharik M L, Finlay B B. Disruption of the Salmonella-containing vacuole leads to increased replication of Salmonella enterica serovar Typhimurium in the cytosol of epithelial cells. Infection and immunity, 2002, p. 3264, Vol. 70, No. 6.
32. Sambrook J, E. F. Fritsch, and T. Maniatis. Molecular Cloning; A Laboratory Manual. Second ed. New York: Cold Spring Harbor Laboratory Press, 1989.
33. Konjufca V, Jenkins M, Wang S, Juarez-Rodriguez M D, Curtiss R, 3rd. Immunogenicity of recombinant attenuated Salmonella enterica serovar Typhimurium vaccine strains carrying a gene that encodes Eimeria tenella antigen SO7. Infect Immun, 2008, pp. 5745-5753, Vol. 76, No. 12.
34. UK. L. Cleavage of structural proteins during the assembly of the head of bacteriophage T4. Nature, 1970, pp. 680-685, Vol. 227, No. 5259.
35. McCown M F, Pekosz A. The influenza A virus M2 cytoplasmic tail is required for infectious virus production and efficient genome packaging. J Virol, 2005, pp. 3595-3605, Vol. 79, No. 6.
36. McCown M F, Pekosz A. Distinct domains of the influenza a virus M2 protein cytoplasmic tail mediate binding to the M1 protein and facilitate infectious virus production. J Virol, 2006, pp. 8178-8189, Vol. 80, No. 16.
37. Curtiss S R, 3rd. Chromosomal Aberrations Associated With Mutations To Bacteriophage Resistance In Escherichia Coli. J Bacteriol, 1965, pp. 28-40, Vol. 89.
38. Sedgwick J D, and P. G. Holt. A solid-phase immunoenzymatic technique for the enumeration of specific antibody-secreting cells. J Immunol Methods, 1983, pp. 301-309, Vol. 57.
39. Tao P, Luo M, Pan R, Ling D, Zhou S, Tien P, et al. Enhanced protective immunity against H5N1 influenza virus challenge by vaccination with DNA expressing a chimeric hemagglutinin in combination with an MHC class I-restricted epitope of nucleoprotein in mice. Antiviral Research, 2009, pp. 253-260, Vol. 81, No. 3.
40. Epstein S L, Tumpey T M, Misplon J A, Lo C Y, Cooper L A, Subbarao K, et al. DNA vaccine expressing conserved influenza virus proteins protective against H5N1 challenge infection in mice. 2002.
41. Bodmer H C, Pemberton R M, Rothbard J B, Askonas B A. Enhanced recognition of a modified peptide antigen by cytotoxic T cells specific for influenza nucleoprotein. Cell, 1988, p. 253, Vol. 52, No. 2.
42. Deliyannis G, Jackson D C, Ede N J, Zeng W, Hourdakis I, Sakabetis E, et al. Induction of long-term memory CD8+ T cells for recall of viral clearing responses against influenza virus. The Journal of Virology, 2002, p. 4212, Vol. 76, No. 9.
43. Doherty P C, Kelso A. Toward a broadly protective influenza vaccine. The Journal of Clinical Investigation, 2008, p. 3273, Vol. 118, No. 10.
44. De Boer G F, Back W, Osterhaus A. An ELISA for detection of antibodies against influenza A nucleoprotein in humans and various animal species. Archives of virology, 1990, pp. 47-67, Vol. 115, No. 1.
45. Gerdil C. The annual production cycle for influenza vaccine. Vaccine, 2003, pp. 1776-1779, Vol. 21, No. 16.
46. Andrew M E, Coupar B E, Boyle D B, Ada G L. The roles of influenza virus haemagglutinin and nucleoprotein in protection: analysis using vaccinia virus recombinants. Scand J Immunol, 1987, pp. 21-28, Vol. 25, No. 1.
47. Carragher D M, Kaminski D A, Moquin A, Hartson L, Randall T D. A novel role for non-neutralizing antibodies against nucleoprotein in facilitating resistance to influenza virus. J Immunol, 2008, pp. 4168-4176, Vol. 181, No. 6.
48. Shahzad M I, Naeem K, Mukhtar M, Khanum A. Passive immunization against highly pathogenic Avian Influenza Virus (AIV) strain H7N3 with antiserum generated from viral polypeptides protect poultry birds from lethal viral infection. Virol J, 2008, p. 144, No. 5.

### Example 6

### Expand the Antigens to Include Elements of the Influenza Virus that Display Heterosubtypic Conservation to Provide Protection Despite Antigenic Shift/Antigenic Drift and to Increase the CTL Response and Generate Memory T Cells for Lifelong Immunity.

### Bacterial Strains, Plasmids, and Primers

The bacterial strains and plasmids used in this example are listed in Table 4. Serovar Typhimurium strains are derived from the highly virulent strain UK-1.

TABLE 4
Bacterial strains and plasmids used in this study

| Strain or | Genotype or relevant | Source or |
|---|---|---|
| plasmid | characteristics | reference |
| Strains | | |
| S. enterica | | |
| serovar | | |
| Typhimurium | | |
| χ11246 | ΔasdA27::TT araC P_{BAD} c2ΔaraBAD23 | See Table 1 |
| | Δ(gmd-fcl)-26 Δpmi- | |
| | 2426ΔrelA198::araC P_{BAD} lacITT | |
| | ΔP_{murA25}::TT araC P_{BAD} murA ΔsifA | |
| χ11509 | ΔasdA27::TT araC P_{BAD} c2ΔaraBAD23 | This |
| | Δ(wza-wcaM)-8 Δpmi- | invention |
| | 2426ΔrelA198::araC P_{BAD} lacITT | |
| | ΔP_{murA25}::TT araC P_{BAD} murA ΔsifA | |
| Plasmids | | |
| pYA5121 | Lysis vector pYA3681 carrying | This |
| | updated codon-optimized NP gene | invention |
| pYA5122 | Lysis vector pYA3681 carrying | This |
| | sequence encoding P_{trc}-Opt-HAₐ-AAY- | invention |
| | Opt-HA_{b} (cassette of HA T cell epitope | |
| | tag) | |
| pYA5126 | Lysis vector pYA3681 carrying | This |
| | updated codon-optimized NP gene | invention |
| | with encoded C-terminal in-frame | |
| | fused Opt + HAₐ-AAY-Opt-HA_{b} | |
| pYA SopE2₁₋₈₀ + | Lysis vector pYA3681 carriying codon- | This |
| Opt-NP | optimized NP gene (updated) with | invention |
| | encoded N-terminal in-frame fused | |
| | SopE2 N-terminal 1-80 amino acids | |
| pYA SopE2₁₋₈₀ + | Lysis vector pYA3681 carrying codon- | This |
| Opt-NP + Opt- | optimized NP gene (updated) with | invention |
| HAₐ-AAY-Opt- | encoded N-terminal in-frame fused | |
| HA_{b} | SopE2 N-terminal 1-80 amino acids | |
| | and C-terminal in-frame fused Opt- | |
| | HAₐ-AAY-Opt-HA_{b} | |

### Strain Construction and Characterization.

To ensure lysis occurring in vivo, we include the deletion mutation of Δ(wza-wcaM)-8. The mutation Δ(wza-wcaM)-8 deletes twenty structural genes from wza to wcaM that encode colanic acid synthesis genes, thus blocking colanic acid production. The inability to synthesize colanic acid reduces the ability of Salmonella to form biofilms and thus contributes to biological containment and lessens the likelihood for adherence to gallstones, thus reducing persistence. The Δ(wza-wcaM)-8 mutation was introduced into a regulated delayed lysis strain to obtain strain χ11509 (Table 4).

The regulated lysis phenotype of bacterial strains was confirmed by diluting overnight cultures 10⁻³ and 10⁴ and plating 100 μl on LB only and LB containing 0.2% arabinose plates and incubating at 37° C. The lysis strains grow on LB containing arabinose plates only depicting complete dependence on the presence of arabinose for survival.

### Updated Codon Optimization of NP Gene.

The sequence of the codon optimized nucleoprotein (NP) gene of Influenza virus strain A/WSN/33 was amplified from plasmid pUC-57-NP-WSN (Genscript) and the short additional sequences in the N-terminal and C-terminal ends of the NP gene were removed. This resulted in the cassette of the updated codon optimized NP gene (uOpt-NP) (49).

### Codon Optimization of HA T-Cell Epitope Tag.

To determine the sequence of HA-epitope tag from conserved T cell epitopes of influenza A virus HA antigen for NP-HA-tag fusion constructs, we searched the HA T cell epitopes with criteria of influenza A, haemagglutinin, and T cell responses against immune epitope database at http://www.immuneepitope.orq/ (supported by the NIAID). Many duplicated epitopes were found among 184 positive T cell assays. We exported a list of all linear peptide sequences and the BLAST was performed using each unique sequence to determine the number of aligning sequences (by conservation). Sequence alignment was done with strains of interest (Avian/swine/recent WHO recommended vaccine strains) (FIG. 15). Two peptides (HAₐ and HA_{b}) for heterosubtypic conservation (aligning with >500 database sequences) were selected (FIG. 16). Also these two peptides both fall into the HA2 portion of the HA molecule since the epitopes in stalk, transmembrane, and cytoplasmic region are more conserved. The codon optimized DNA sequences of two proposed epitopes (HAₐ and HA_{b}) were linked by an AAY linker since it was known to contribute to proteasome processing of linear epitopes when placed directly C-terminal to the desired cleavage site (50). The resulting HA epitope tag was named Opt-HAₐ-AAY-Opt-HA_{b} (FIG. 15).

### Vector Construction.

Plasmid pYA5121.

The updated codon optimized NP gene (uOpt-NP) was inserted into lysis vector pYA3681 at the NcoI site to create plasmid pYA5121 (FIG. 14). The DNA sequence of plasmid pYA5121 is listed in Table 5.

Plasmid pYA5122.

The codon optimized DNA sequences of HA epitope tag Opt-HAₐ-AAY-Opt-HA_{b} was inserted into the lysis vector pYA3681 using SphI and PciI sites to create plasmid pYA5122 (FIG. 17). The DNA sequence of plasmid pYA5122 is listed in Table 6.

Plasmid pYA5126.

The updated codon-optimized NP gene with C-terminal in-frame fused HA T cell epitope tag (Opt-HAₐ-AAY-Opt-HA_{b}) was inserted into lysis vector pYA3681 using NcoI and PciI sites to create plasmid pYA5126 (FIG. 18). The DNA sequence of plasmid pYA5126 is listed in Table 7.

### Plasmid Stability.

Plasmids pYA 3681, pYA5121, pYA5122, and pYA5126 were transformed into RASV strains χ11246 and χ11509, respectively. Plasmid stability was determined. RASV strains were grown overnight in 3 ml cultures supplemented with 50 μg/ml of DAP and 0.2% arabinose. Next day, fresh LB supplemented with DAP and arabinose was inoculated with a 1:1000 dilution of each overnight culture and grown statically at 37° C. overnight (about 14 hours). To estimate the proportions of bacterial cells retaining the Asd⁺ plasmid, cultures were serially diluted and 10⁻⁵ and 10⁻⁶ were plated on LB plates supplemented with DAP and arabinose and grown overnight at 37° C. Next day, 100 colonies from these plates were picked and patched onto LB supplemented with arabinose and LB supplemented with DAP and arabinose. Percentage of clones retaining the plasmids was determined by counting the colonies. Concurrently, colonies from each day's plating were grown in fresh LB supplemented with DAP and arabinose and the process repeated for 5 consecutive days (50 generations).

### SDS-PAGE and Immunoblots.

To evaluate the synthesis of NP protein and NP-HA-tag fusion protein from plasmids pYA5121 and pYA5126 in Salmonella strains χ11246 and χ11509, the bacterial cells were grown overnight at 37° C. in LB containing 0.2% arabinose. The cultures were diluted 1:100 in fresh LB containing 0.2% arabinose and grown rotating at 37° C. till the optical density (O.D) of 0.6 at 600 nm by spectrophotometer was achieved. The synthesis of NP protein and NP-HA-tag fusion protein was induced by adding 0.3 mM IPTG into the bacterial cultures. Aliquots of samples (1 ml) were taken, centrifuged at low speed to pellet down the bacteria, resuspended in 2×SDS-PAGE loading buffer and boiled for 10 min in a water bath. The samples were centrifuged for 10 min, diluted 1:10 in 2× sample loading buffer and 10 μl loaded in 12.5% SDS-PAGE gels and separated by electrophoresis. Samples were transferred to nitrocellulose membranes and blocked with 5% skimmed milk for 1 hour at room temperature. The membranes were rinsed with PBS-0.05% Tween (T-20) three times and incubated with mouse monoclonal anti-Influenza A NP antibody (Abcam) for 1 hour with constant shaking. After washing with PBS-T20 as before, the membranes were incubated with anti-mouse conjugate-alkaline phosphatase for 1 hour and developed with nitroblue tetrazolium-5-bromo-4-chloro-3-indolyphosphate (BCIP) (Sigma, St. Louis, Mo.). The membranes were washed with water and airdired. The LacI regulated synthesis of NP protein and NP-HA-tag fusion protein from plasmids pYA5121 and pYA5126 in Salmonella strains χ11246 and χ11509 was shown in FIG. 19 and FIG. 20, respectively.

### Improve NP and NP HA-Tag Constructs.

Salmonella SopE2, an invasion- and virulence-associated type III secreted protein (51), was found to be very rapidly ubiquitinated to facilitate antigen movement to the proteosome for efficient MHC Class I presentation (52). To enhance MHC Class I presentation of NP and NP-HA tag proteins, SopE2 N-terminal 1-80 amino acids may be fused to the N-terminal ends of NP and NP-HA tag proteins.

Design and Construction of Plasmid pYA SopE2₁₋₈₀+uOpt-NP.

A cassette of updated codon-optimized NP gene with N-terminal in-frame fused SopE2 N-terminal 1-80 amino acids may be inserted into the lysis vector pYA3681 to maximize expression in S. Typhimurium and for efficient MHC Class I presentation (FIG. 21).

Design and Construction of Plasmid pYA SopE2₁₋₈₀+uOpt-NP+Opt-HAₐ-AAY-Opt-HA_{b}.

A cassette of updated codon-optimized NP gene with N-terminal in-frame fused to the SopE2 N-terminal 1-80 amino acids and C-terminal in-frame fused Opt-HAₐ-AAY-Opt-HA_{b} is being inserted into the lysis vector pYA3681 for maximal expression in S. Typhimurium and efficient MHC Class I presentation (FIG. 22).

### Development of Protocol for Antigen Specific T-Cell Assay Based on the Method of TRAP Assay.

To “separate” the immune response to Salmonella from the immune response to influenza antigens, we are exploring a new method based on the TRAP assay (T cell recognition of APCs by protein capture or trogocytosis analysis protocol) assay (53, 54). TRAP assay is based on a process carried out by CD4⁺ T cells, CD8⁺ T cells, and B lymphocytes called trogocytosis. Trogocytosis, as it has been described, is a process by which lymphocytes capture fragments of the plasma membrane from the antigen-presenting cells (APCs) expressing their cognate antigen. For this method, a label (such as a fluorescent lipid or biotin) is first incorporated in the membrane of APCs. These labeled cells are then co-cultured for a few hours with a population of cells containing the lymphocytes isolated from immunized mice. After this period of stimulation, lymphocytes that have performed trogocytosis are identified by their acquisition of the label initially present on the APC membrane using flow cytometry.

### Immunization Experiments

The vaccine strains χ11246(pYA5121), χ11246(pYA5122), χ11246(pYA5126), χ11246(pYA3681), χ11509(pYA5121), χ11509(pYA5122), χ11509(pYA5126), χ11509(pYA3681), χ11246(pYA SopE2₁₋₈₀+uOpt-NP), χ11246(pYA SopE2₁₋₈₀+uOpt-NP+Opt-HAₐ-AAY-Opt-HA_{b}), χ11509(pYA SopE2₁₋₈₀+uOpt-NP), and χ11509(pYA SopE2₁₋₈₀+uOpt-NP+Opt-HAₐ-AAY-Opt-HA_{b}) may be grown as described in the Materials and Methods for Examples 1 to 5 and used to orally immunize female BALB/c mice. Immune protection to challenge with Influenza virus may be superior in strains displaying regulated delayed cell lysis that can escape the endosome compared to those recombinant vaccine strains unable to escape the endosome. Based of the teachings in Examples 1 to 5 that the delivery of HA epitopes in addition to NP to the cytosol by vaccine escape from the endosome followed by lysis in the cytosol may further augment induction of protective immunity against influenza challenge. It is also expected that fusion of the SopE2 fragment that should be rapidly ubiquinated to facilitate trafficking to the proteosome for class I presentation to further magnify CD8 T cell responses and the level of protective immunity. Overall, these approaches to inducing protective T cell immune responses against bacterial, viral and parasite pathogens are superior to other methods of antigen delivery that are often constrained by antigen structural constraints reducing or even precluding secretion as by the T3SS into the host cell cytosol.

TABLE 5
DNA sequence of plasmid pYA5121.

```
Range: 1 to 7320
    50
AGATCTAGCC CGCCTAATGA GCGGGCTTTT TTTTAATTCG
CAATTCCCCG
  100
ATGCATAATG TGCCTGTCAA ATGGACGAAG CAGGGATTCT
GCAAACCCTA
  150
TGCTACTCCG TCAAGCCGTC AATTGTCTGA TTCGTTACCA
ATTATGACAA
  200
CTTGACGGCT ACATCATTCA CTTTTTCTTC ACAACCGGCA
CGGAACTCGC
  250
TCGGGCTGGC CCCGGTGCAT TTTTTAAATA CCCGCGAGAA
ATAGAGTTGA
  300
TCGTCAAAAC CAACATTGCG ACCGACGGTG GCGATAGGCA
TCCGGGTGGT
  350
GCTCAAAAGC AGCTTCGCCT GGCTGATACG TTGGTCCTCG
CGCCAGCTTA
  400
AGACGCTAAT CCCTAACTGC TGGCGGAAAA GATGTGACAG
ACGCGACGGC
  450
GACAAGCAAA CATGCTGTGC GACGCTGGCG ATATCAAAAT
TGCTGTCTGC
  500
CAGGTGATCG CTGATGTACT GACAAGCCTC GCGTACCCGA
TTATCCATCG
  550
GTGGATGGAG CGACTCGTTA ATCGCTTCCA TGCGCCGCAG
TAACAATTGC
  600
TCAAGCAGAT TTATCGCCAG CAGCTCCGAA TAGCGCCCTT
CCCCTTGCCC
  650
GGCGTTAATG ATTTGCCCAA ACAGGTCGCT GAAATGCGGC
TGGTGCGCTT
  700
CATCCGGGCG AAAGAACCCC GTATTGGCAA ATATTGACGG
CCAGTTAAGC
  750
CATTCATGCC AGTAGGCGCG CGGACGAAAG TAAACCCACT
GGTGATACCA
  800
TTCGCGAGCC TCCGGATGAC GACCGTAGTG ATGAATCTCT
CCTGGCGGGA
  850
ACAGCAAAAT ATCACCCGGT CGGCAAACAA ATTCTCGTCC
CTGATTTTTC
  900
ACCACCCCCT GACCGCGAAT GGTGAGATTG AGAATATAAC
CTTTCATTCC
  950
CAGCGGTCGG TCGATAAAAA AATCGAGATA ACCGTTGGCC
TCAATCGGCG
1000
TTAAACCCGC CACCAGATGG GCATTAAACG AGTATCCCGG
CAGCAGGGGA
1050
TCATTTTGCG CTTCAGCCAT ACTTTTCATA CTCCCGCCAT
TCAGAGAAGA
1100
AACCAATTGT CCATATTGCA TCAGACATTG CCGTCACTGC
GTCTTTTACT
1150
GGCTCTTCTC GCTAACCAAA CCGGTAACCC CGCTTATTAA
AAGCATTCTG
1200
TAACAAAGCG GGACCAAAGC CATGACAAAA ACGCGTAACA
AAAGTGTCTA
1250
TAATCACGGC AGAAAAGTCC ACATTGATTA TTTGCACGGC
GTCACACTTT
1300
GCTATGCCAT AGCATTTTTA TCCATAAGAT TAGCGGATCC
TACCTGACGC
1350
TTTTTATCGC AACTCTCTAC TGTTTCTCCA TACCCGTTTT
TTTGGGCTAG
1400
CGAATTCTGA GAACAAACTA AATGGATAAA TTTCGTGTTC
AGGGGCCAAC
1450
GAAGCTCCAG GGCGAAGTCA CAATTTCCGG CGCTAAAAAT
GCTGCTCTGC
1500
CTATCCTTTT TGCCGCACTA CTGGCGGAAG AACCGGTAGA
GATCCAGAAC
1550
GTCCCGAAAC TGAAAGACGT CGATACATCA ATGAAGCTGC
TAAGCCAGCT
1600
GGGTGCGAAA GTAGAACGTA ATGGTTCTGT GCATATTGAT
GCCCGCGACG
1650
TTAATGTATT CTGCGCACCT TACGATCTGG TTAAAACCAT
GCGTGCTTCT
1700
ATCTGGGCGC TGGGGCCGCT GGTAGCGCGC TTTGGTCAGG
GGCAAGTTTC
1750
ACTACCTGGC GGTTGTACGA TCGGTGCGCG TCCGGTTGAT
CTACACATTT
1800
CTGGCCTCGA ACAATTAGGC GCGACCATCA AACTGGAAGA
AGGTTACGTT
1850
AAAGCTTCCG TCGATGGTCG TTTGAAAGGT GCACATATCG
TGATGGATAA
1900
AGTCAGCGTT GGCGCAACGG TGACCATCAT GTGTGCTGCA
ACCCTGGCGG
1950
AAGGCACCAC GATTATTGAA AACGCAGCGC GTGAACCGGA
AATCGTCGAT
2000
ACCGCGAACT TCCTGATTAC GCTGGGTGCG AAAATTAGCG
GTCAGGGCAC
2050
CGATCGTATC GTCATCGAAG GTGTGGAACG TTTAGGCGGC
GGTGTCTATC
2100
GCGTTCTGCC GGATCGTATC GAAACCGGTA CTTTCCTGGT
GGCGGCGGCG
2150
ATTTCTCGCG GCAAAATTAT CTGCCGTAAC GCGCAGCCAG
ATACTCTCGA
2200
CGCCGTGCTG GCGAAACTGC GTGACGCTGG AGCGGACATC
GAAGTCGGCG
2250
AAGACTGGAT TAGCCTGGAT ATGCATGGCA AACGTCCGAA
GGCTGTTAAC
2300
GTACGTACCG CGCCGCATCC GGCATTCCCG ACCGATATGC
AGGCCCAGTT
2350
CACGCTGTTG AACCTGGTGG CAGAAGGGAC CGGGTTTATC
ACCGAAACGG
2400
TCTTTGAAAA CCGCTTTATG CATGTGCCAG AGCTGAGCCG
TATGGGCGCG
2450
CACGCCGAAA TCGAAAGCAA TACCGTTATT TGTCACGGTG
TTGAAAAACT
2500
TTCTGGCGCA CAGGTTATGG CAACCGATCT GCGTGCATCA
GCAAGCCTGG
2550
TGCTGGCTGG CTGTATTGCG GAAGGGACGA CGGTGGTTGA
TCGTATTTAT
2600
CACATCGATC GTGGCTACGA ACGCATTGAA GACAAACTGC
GCGCTTTAGG
2650
TGCAAATATT GAGCGTGTGA AAGGCGAATA AGAATTCAGG
AAAAAAACGC
2700
TGTGAAAAAT GTTGGTTTTA TCGGCTGGCG CGGAATGGTC
GGCTCTGTTC
2750
TCATGCAACG CATGGTAGAG GAGCGCGATT TCGACGCTAT
TCGCCCTGTT
2800
TTCTTTTCTA CCTCCCAGTT TGGACAGGCG GCGCCCACCT
TCGGCGACAC
2850
CTCCACCGGC ACGCTACAGG ACGCTTTTGA TCTGGATGCG
CTAAAAGCGC
2900
TCGATATCAT CGTGACCTGC CAGGGCGGCG ATTATACCAA
CGAAATTTAT
2950
CCAAAGCTGC GCGAAAGCGG ATGGCAGGGT TACTGGATTG
ATGCGGCTTC
3000
TACGCTGCGC ATGAAAGATG ATGCCATTAT TATTCTCGAC
CCGGTCAACC
3050
AGGACGTGAT TACCGACGGC CTGAACAATG GCGTGAAGAC
CTTTGTGGGC
3100
GGTAACTGTA CCGTTAGCCT GATGTTGATG TCGCTGGGCG
GTCTCTTTGC
3150
CCATAATCTC GTTGACTGGG TATCCGTCGC GACCTATCAG
GCCGCCTCCG
3200
GCGGCGGCGC GCGCCATATG CGCGAGCTGT TAACCCAGAT
GGGTCAGTTG
3250
TATGGCCATG TCGCCGATGA ACTGGCGACG CCGTCTTCCG
CAATTCTTGA
3300
TATTGAACGC AAAGTTACGG CATTGACCCG CAGCGGCGAG
CTGCCGGTTG
3350
ATAACTTTGG CGTACCGCTG GCGGGAAGCC TGATCCCCTG
GATCGACAAA
3400
CAGCTCGATA ACGGCCAGAG CCGCGAAGAG TGGAAAGGCC
AGGCGGAAAC
3450
CAACAAGATT CTCAATACTG CCTCTGTGAT TCCGGTTGAT
GGTTTGTGTG
3500
TGCGCGTCGG CGCGCTGCGC TGTCACAGCC AGGCGTTCAC
CATCAAGCTG
3550
AAAAAAGAGG TATCCATTCC GACGGTGGAA GAACTGCTGG
CGGCACATAA
3600
TCCGTGGGCG AAAGTGGTGC CGAACGATCG TGATATCACT
ATGCGCGAAT
3650
TAACCCCGGC GGCGGTGACC GGCACGTTGA CTACGCCGGT
TGGTCGTCTG
3700
CGTAAGCTGA ACATGGGGCC AGAGTTCTTG TCGGCGTTTA
CCGTAGGCGA
3750
CCAGTTGTTA TGGGGCGCCG CCGAGCCGCT GCGTCGAATG
CTGCGCCAGT
3800
TGGCGTAGTC TAGCTGCACG ATACCGTCGA CTTGTACATA
GACTCGCTCC
3850
GAAATTAAAG AACACTTAAA TTATCTACTA AAGGAATCTT
TAGTCAAGTT
3900
TATTTAAGAT GACTTAACTA TGAATACACA ATTGATGGGT
GAGCGTAGGA
3950
GCATGCTTAT GCGAAAGGCC ATCCTGACGG ATGGCCTTTT
TGGATCTTCC
4000
GGAAGACCTT CCATTCTGAA ATGAGCTGTT GACAATTAAT
CATCCGGCTC
4050
GTATAATGTG TGGAATTGTG AGCGGATAAC AATTTCACAC
AGGAAACAGA
4100
CCATGGCGAC CAAAGGCACC AAACGTAGCT ATGAACAGAT
GGAAACCGAT
4150
GGCGAACGTC AGAACGCGAC CGAAATTCGT GCGAGCGTGG
GCAAAATGAT
4200
TGATGGCATT GGCCGTTTTT ATATTCAGAT GTGCACCGAA
CTGAAACTGA
4250
GCGATTATGA AGGCCGTCTG ATTCAGAACA GCCTGACCAT
TGAACGTATG
4300
GTGCTGAGCG CGTTTGATGA ACGTCGTAAC AAATATCTGG
AAGAACATCC
4350
GAGCGCGGGC AAAGATCCAA AGAAAACCGG CGGCCCGATT
TATCGTCGTG
4400
TGGATGGCAA ATGGCGTCGT GAACTGATTC TGTATGATAA
AGAAGAAATT
4450
CGTCGTATTT GGCGTCAGGC GAACAACGGC GATGATGCGA
CCGCGGGCCT
4500
GACCCACATG ATGATTTGGC ATAGCAACCT GAACGATGCG
ACCTATCAGC
4550
GTACCCGTGC GCTGGTGCGT ACCGGCATGG ACCCACGTAT
GTGCAGCCTG
4600
ATGCAGGGCA GCACCCTGCC GCGTCGTAGC GGTGCAGCAG
GTGCAGCAGT
4650
GAAAGGCGTG GGTACGATGG TGATGGAACT GATTCGTATG
ATTAAACGTG
4700
GCATTAACGA TCGTAACTTT TGGCGTGGCG AAAACGGCCG
TCGTACCCGT
4750
ATTGCGTATG AACGTATGTG CAACATTCTG AAAGGCAAAT
TTCAGACCGC
4800
GGCGCAGCGT ACGATGGTGG ATCAAGTGCG TGAAAGCCGT
AACCCGGGCA
4850
ACGCGGAATT TGAAGACCTG ATTTTTCTGG CGCGTAGCGC
GCTGATTCTG
4900
CGTGGCAGCG TGGCGCATAA AAGCTGCCTG CCGGCGTGCG
TGTATGGCCC
4950
GGCGGTGGCG AGCGGCTATG ATTTTGAACG TGAAGGCTAT
AGCCTGGTGG
5000
GCATTGATCC GTTTCGTCTG CTGCAGAACA GCCAGGTGTA
TAGCCTGATT
5050
CGTCCGAACG AAAACCCGGC GCATAAAAGC CAGCTGGTGT
GGATGGCGTG
5100
CCATAGCGCG GCGTTTGAAG ACCTGCGTGT GAGCAGCTTT
ATTCGTGGCA
5150
CCAAAGTGGT GCCGCGTGGC AAACTGAGCA CCCGTGGCGT
GCAGATTGCG
5200
AGCAACGAAA ACATGGAAAC GATGGAAAGC AGCACCCTGG
AACTGCGTAG
5250
CCGTTATTGG GCGATTCGTA CCCGTAGCGG CGGCAACACC
AACCAGCAGC
5300
GTGCGAGCAG CGGCCAGATT AGCATTCAGC CGACCTTTAG
CGTGCAGCGT
5350
AACCTGCCGT TTGATCGTCC GACCATTATG GCGGCGTTTA
CCGGCAACAC
5400
CGAAGGCCGT ACCAGCGATA TGCGTACCGA AATTATTCGT
CTGATGGAAA
5450
GCGCGCGTCC GGAAGATGTG AGCTTTCAGG GCCGTGGCGT
GTTTGAACTG
5500
AGCGATGAAA AAGCGACCAG CCCGATTGTG CCGAGCTTTG
ATATGAGCAA
5550
CGAAGGCAGC TACTTTTTCG GCGATAACGC GGAAGAATAT
GATAACTAAG
5600
ACCCATGGGA ATTCGCAATT CCCGGGGATC CGTCGACCTG
CAGCCAAGCT
5650
CCCAAGCTTG GCTGTTTTGG CGGATGAGAG AAGATTTTCA
GCCTGATACA
5700
GATTAAATCA GAACGCAGAA GCGGTCTGAT AAAACAGAAT
TTGCCTGGCG
5750
GCAGTAGCGC GGTGGTCCCA CCTGACCCCA TGCCGAACTC
AGAAGTGAAA
5800
CGCCGTAGCG CCGATGGTAG TGTGGGGTCT CCCCATGCGA
GAGTAGGGAA
5850
CTGCCAGGCA TCAAATAAAA CGAAAGGCTC AGTCGAAAGA
CTGGGCCTTT
5900
CGTTTTATCT GTTGTTTGTC GGTGAACGCT CTCCTGAGTA
GGACAAATCC
5950
GCCGGGAGCG GATTTGAACG TTGCGAAGCA ACGGCCCGGA
GGGTGGCGGG
6000
CAGGACGCCC GCCATAAACT GCCAGGCATC AAATTAAGCA
GAAGGCCATC
6050
CTGACGGATG GCCTTTTTGC GTTTCTACAA ACTCTTTTGT
TTATTTTTCT
6100
AAATACATTC AAATATGTAT CCGCTCATGA GACAATAACC
CTGATAAATG
6150
CTTCAATAAT GGAAGATCTT CCAACATCAC AGGTAAACAG
AAACGTCGGG
6200
TCGATCGGGA AATTCTTTCC CGGACGGCGC GGGGTTGGGC
AAGCCGCAGG
6250
CGCGTCAGTG CTTTTAGCGG GTGTCGGGGC GCAGCCATGA
CCCAGTCACG
6300
TAGCGATAGC GGAGTGTATA CTGGCTTAAC TATGCGGCAT
CAGAGCAGAT
6350
TGTACTGAGA GTGCACCATA TGCGGTGTGA AATACCGCAC
AGATGCGTAA
6400
GGAGAAAATA CCGCATCAGG CGCTCTTCCG CTTCCTCGCT
CACTGACTCG
6450
CTGCGCTCGG TCGTTCGGCT GCGGCGAGCG GTATCAGCTC
ACTCAAAGGC
6500
GGTAATACGG TTATCCACAG AATCAGGGGA TAACGCAGGA
AAGAACATGT
6550
GAGCAAAAGG CCAGCAAAAG GCCAGGAACC GTAAAAAGGC
CGCGTTGCTG
6600
GCGTTTTTCC ATAGGCTCCG CCCCCCTGAC GAGCATCACA
AAAATCGACG
6650
CTCAAGTCAG AGGTGGCGAA ACCCGACAGG ACTATAAAGA
TACCAGGCGT
6700
TTCCCCCTGG AAGCTCCCTC GTGCGCTCTC CTGTTCCGAC
CCTGCCGCTT
6750
ACCGGATACC TGTCCGCCTT TCTCCCTTCG GGAAGCGTGG
CGCTTTCTCA
6800
TAGCTCACGC TGTAGGTATC TCAGTTCGGT GTAGGTCGTT
CGCTCCAAGC
6850
TGGGCTGTGT GCACGAACCC CCCGTTCAGC CCGACCGCTG
CGCCTTATCC
6900
GGTAACTATC GTCTTGAGTC CAACCCGGTA AGACACGACT
TATCGCCACT
6950
GGCAGCAGCC ACTGGTAACA GGATTAGCAG AGCGAGGTAT
GTAGGCGGTG
7000
CTACAGAGTT CTTGAAGTGG TGGCCTAACT ACGGCTACAC
TAGAAGGACA
7050
GTATTTGGTA TCTGCGCTCT GCTGAAGCCA GTTACCTTCG
GAAAAAGAGT
7100
TGGTAGCTCT TGATCCGGCA AACAAACCAC CGCTGGTAGC
GGTGGTTTTT
7150
TTGTTTGCAA GCAGCAGATT ACGCGCAGAA AAAAAGGATC
TCAAGAAGAT
7200
CCTTTGATCT TTTCTACGGG GTCTGACGCT CAGTGGAACG
AAAACTCACG
7250
TTAAGGGATT TTGGTCATGA GATTATCAAA AAGGATCTTC
ACCTAGATCC
7300
TTTTAAATTA AAAATGAAGT TTTAAATCAA TCTAAAGTAT
ATATGAGTAA ACTTGGTCTG ACAGTCTAGA
```

TABLE 6
DNA sequence of plasmid pYA5122.

```
Range: 1 to 7320
    50
AGATCTAGCC CGCCTAATGA GCGGGCTTTT TTTTAATTCG
CAATTCCCCG
  100
ATGCATAATG TGCCTGTCAA ATGGACGAAG CAGGGATTCT
GCAAACCCTA
  150
TGCTACTCCG TCAAGCCGTC AATTGTCTGA TTCGTTACCA
ATTATGACAA
  200
CTTGACGGCT ACATCATTCA CTTTTTCTTC ACAACCGGCA
CGGAACTCGC
  250
TCGGGCTGGC CCCGGTGCAT TTTTTAAATA CCCGCGAGAA
ATAGAGTTGA
  300
TCGTCAAAAC CAACATTGCG ACCGACGGTG GCGATAGGCA
TCCGGGTGGT
  350
GCTCAAAAGC AGCTTCGCCT GGCTGATACG TTGGTCCTCG
CGCCAGCTTA
  400
AGACGCTAAT CCCTAACTGC TGGCGGAAAA GATGTGACAG
ACGCGACGGC
  450
GACAAGCAAA CATGCTGTGC GACGCTGGCG ATATCAAAAT
TGCTGTCTGC
  500
CAGGTGATCG CTGATGTACT GACAAGCCTC GCGTACCCGA
TTATCCATCG
  550
GTGGATGGAG CGACTCGTTA ATCGCTTCCA TGCGCCGCAG
TAACAATTGC
  600
TCAAGCAGAT TTATCGCCAG CAGCTCCGAA TAGCGCCCTT
CCCCTTGCCC
  650
GGCGTTAATG ATTTGCCCAA ACAGGTCGCT GAAATGCGGC
TGGTGCGCTT
  700
CATCCGGGCG AAAGAACCCC GTATTGGCAA ATATTGACGG
CCAGTTAAGC
  750
CATTCATGCC AGTAGGCGCG CGGACGAAAG TAAACCCACT
GGTGATACCA
  800
TTCGCGAGCC TCCGGATGAC GACCGTAGTG ATGAATCTCT
CCTGGCGGGA
  850
ACAGCAAAAT ATCACCCGGT CGGCAAACAA ATTCTCGTCC
CTGATTTTTC
  900
ACCACCCCCT GACCGCGAAT GGTGAGATTG AGAATATAAC
CTTTCATTCC
  950
CAGCGGTCGG TCGATAAAAA AATCGAGATA ACCGTTGGCC
TCAATCGGCG
1000
TTAAACCCGC CACCAGATGG GCATTAAACG AGTATCCCGG
CAGCAGGGGA
1050
TCATTTTGCG CTTCAGCCAT ACTTTTCATA CTCCCGCCAT
TCAGAGAAGA
1100
AACCAATTGT CCATATTGCA TCAGACATTG CCGTCACTGC
GTCTTTTACT
1150
GGCTCTTCTC GCTAACCAAA CCGGTAACCC CGCTTATTAA
AAGCATTCTG
1200
TAACAAAGCG GGACCAAAGC CATGACAAAA ACGCGTAACA
AAAGTGTCTA
1250
TAATCACGGC AGAAAAGTCC ACATTGATTA TTTGCACGGC
GTCACACTTT
1300
GCTATGCCAT AGCATTTTTA TCCATAAGAT TAGCGGATCC
TACCTGACGC
1350
TTTTTATCGC AACTCTCTAC TGTTTCTCCA TACCCGTTTT
TTTGGGCTAG
1400
CGAATTCTGA GAACAAACTA AATGGATAAA TTTCGTGTTC
AGGGGCCAAC
1450
GAAGCTCCAG GGCGAAGTCA CAATTTCCGG CGCTAAAAAT
GCTGCTCTGC
1500
CTATCCTTTT TGCCGCACTA CTGGCGGAAG AACCGGTAGA
GATCCAGAAC
1550
GTCCCGAAAC TGAAAGACGT CGATACATCA ATGAAGCTGC
TAAGCCAGCT
1600
GGGTGCGAAA GTAGAACGTA ATGGTTCTGT GCATATTGAT
GCCCGCGACG
1650
TTAATGTATT CTGCGCACCT TACGATCTGG TTAAAACCAT
GCGTGCTTCT
1700
ATCTGGGCGC TGGGGCCGCT GGTAGCGCGC TTTGGTCAGG
GGCAAGTTTC
1750
ACTACCTGGC GGTTGTACGA TCGGTGCGCG TCCGGTTGAT
CTACACATTT
1800
CTGGCCTCGA ACAATTAGGC GCGACCATCA AACTGGAAGA
AGGTTACGTT
1850
AAAGCTTCCG TCGATGGTCG TTTGAAAGGT GCACATATCG
TGATGGATAA
1900
AGTCAGCGTT GGCGCAACGG TGACCATCAT GTGTGCTGCA
ACCCTGGCGG
1950
AAGGCACCAC GATTATTGAA AACGCAGCGC GTGAACCGGA
AATCGTCGAT
2000
ACCGCGAACT TCCTGATTAC GCTGGGTGCG AAAATTAGCG
GTCAGGGCAC
2050
CGATCGTATC GTCATCGAAG GTGTGGAACG TTTAGGCGGC
GGTGTCTATC
2100
GCGTTCTGCC GGATCGTATC GAAACCGGTA CTTTCCTGGT
GGCGGCGGCG
2150
ATTTCTCGCG GCAAAATTAT CTGCCGTAAC GCGCAGCCAG
ATACTCTCGA
2200
CGCCGTGCTG GCGAAACTGC GTGACGCTGG AGCGGACATC
GAAGTCGGCG
2250
AAGACTGGAT TAGCCTGGAT ATGCATGGCA AACGTCCGAA
GGCTGTTAAC
2300
GTACGTACCG CGCCGCATCC GGCATTCCCG ACCGATATGC
AGGCCCAGTT
2350
CACGCTGTTG AACCTGGTGG CAGAAGGGAC CGGGTTTATC
ACCGAAACGG
2400
TCTTTGAAAA CCGCTTTATG CATGTGCCAG AGCTGAGCCG
TATGGGCGCG
2450
CACGCCGAAA TCGAAAGCAA TACCGTTATT TGTCACGGTG
TTGAAAAACT
2500
TTCTGGCGCA CAGGTTATGG CAACCGATCT GCGTGCATCA
GCAAGCCTGG
2550
TGCTGGCTGG CTGTATTGCG GAAGGGACGA CGGTGGTTGA
TCGTATTTAT
2600
CACATCGATC GTGGCTACGA ACGCATTGAA GACAAACTGC
GCGCTTTAGG
2650
TGCAAATATT GAGCGTGTGA AAGGCGAATA AGAATTCAGG
AAAAAAACGC
2700
TGTGAAAAAT GTTGGTTTTA TCGGCTGGCG CGGAATGGTC
GGCTCTGTTC
2750
TCATGCAACG CATGGTAGAG GAGCGCGATT TCGACGCTAT
TCGCCCTGTT
2800
TTCTTTTCTA CCTCCCAGTT TGGACAGGCG GCGCCCACCT
TCGGCGACAC
2850
CTCCACCGGC ACGCTACAGG ACGCTTTTGA TCTGGATGCG
CTAAAAGCGC
2900
TCGATATCAT CGTGACCTGC CAGGGCGGCG ATTATACCAA
CGAAATTTAT
2950
CCAAAGCTGC GCGAAAGCGG ATGGCAGGGT TACTGGATTG
ATGCGGCTTC
3000
TACGCTGCGC ATGAAAGATG ATGCCATTAT TATTCTCGAC
CCGGTCAACC
3050
AGGACGTGAT TACCGACGGC CTGAACAATG GCGTGAAGAC
CTTTGTGGGC
3100
GGTAACTGTA CCGTTAGCCT GATGTTGATG TCGCTGGGCG
GTCTCTTTGC
3150
CCATAATCTC GTTGACTGGG TATCCGTCGC GACCTATCAG
GCCGCCTCCG
3200
GCGGCGGCGC GCGCCATATG CGCGAGCTGT TAACCCAGAT
GGGTCAGTTG
3250
TATGGCCATG TCGCCGATGA ACTGGCGACG CCGTCTTCCG
CAATTCTTGA
3300
TATTGAACGC AAAGTTACGG CATTGACCCG CAGCGGCGAG
CTGCCGGTTG
3350
ATAACTTTGG CGTACCGCTG GCGGGAAGCC TGATCCCCTG
GATCGACAAA
3400
CAGCTCGATA ACGGCCAGAG CCGCGAAGAG TGGAAAGGCC
AGGCGGAAAC
3450
CAACAAGATT CTCAATACTG CCTCTGTGAT TCCGGTTGAT
GGTTTGTGTG
3500
TGCGCGTCGG CGCGCTGCGC TGTCACAGCC AGGCGTTCAC
CATCAAGCTG
3550
AAAAAAGAGG TATCCATTCC GACGGTGGAA GAACTGCTGG
CGGCACATAA
3600
TCCGTGGGCG AAAGTGGTGC CGAACGATCG TGATATCACT
ATGCGCGAAT
3650
TAACCCCGGC GGCGGTGACC GGCACGTTGA CTACGCCGGT
TGGTCGTCTG
3700
CGTAAGCTGA ACATGGGGCC AGAGTTCTTG TCGGCGTTTA
CCGTAGGCGA
3750
CCAGTTGTTA TGGGGCGCCG CCGAGCCGCT GCGTCGAATG
CTGCGCCAGT
3800
TGGCGTAGTC TAGCTGCACG ATACCGTCGA CTTGTACATA
GACTCGCTCC
3850
GAAATTAAAG AACACTTAAA TTATCTACTA AAGGAATCTT
TAGTCAAGTT
3900
TATTTAAGAT GACTTAACTA TGAATACACA ATTGATGGGT
GAGCGTAGGA
3950
GCATGCTTAT GCGAAAGGCC ATCCTGACGG ATGGCCTTTT
TGGATCTTCC
4000
GGAAGACCTT CCATTCTGAA ATGAGCTGTT GACAATTAAT
CATCCGGCTC
4050
GTATAATGTG TGGAATTGTG AGCGGATAAC AATTTCACAC
AGGAAACAGA
4100
CCATGGCGAC CAAAGGCACC AAACGTAGCT ATGAACAGAT
GGAAACCGAT
4150
GGCGAACGTC AGAACGCGAC CGAAATTCGT GCGAGCGTGG
GCAAAATGAT
4200
TGATGGCATT GGCCGTTTTT ATATTCAGAT GTGCACCGAA
CTGAAACTGA
4250
GCGATTATGA AGGCCGTCTG ATTCAGAACA GCCTGACCAT
TGAACGTATG
4300
GTGCTGAGCG CGTTTGATGA ACGTCGTAAC AAATATCTGG
AAGAACATCC
4350
GAGCGCGGGC AAAGATCCAA AGAAAACCGG CGGCCCGATT
TATCGTCGTG
4400
TGGATGGCAA ATGGCGTCGT GAACTGATTC TGTATGATAA
AGAAGAAATT
4450
CGTCGTATTT GGCGTCAGGC GAACAACGGC GATGATGCGA
CCGCGGGCCT
4500
GACCCACATG ATGATTTGGC ATAGCAACCT GAACGATGCG
ACCTATCAGC
4550
GTACCCGTGC GCTGGTGCGT ACCGGCATGG ACCCACGTAT
GTGCAGCCTG
4600
ATGCAGGGCA GCACCCTGCC GCGTCGTAGC GGTGCAGCAG
GTGCAGCAGT
4650
GAAAGGCGTG GGTACGATGG TGATGGAACT GATTCGTATG
ATTAAACGTG
4700
GCATTAACGA TCGTAACTTT TGGCGTGGCG AAAACGGCCG
TCGTACCCGT
4750
ATTGCGTATG AACGTATGTG CAACATTCTG AAAGGCAAAT
TTCAGACCGC
4800
GGCGCAGCGT ACGATGGTGG ATCAAGTGCG TGAAAGCCGT
AACCCGGGCA
4850
ACGCGGAATT TGAAGACCTG ATTTTTCTGG CGCGTAGCGC
GCTGATTCTG
4900
CGTGGCAGCG TGGCGCATAA AAGCTGCCTG CCGGCGTGCG
TGTATGGCCC
4950
GGCGGTGGCG AGCGGCTATG ATTTTGAACG TGAAGGCTAT
AGCCTGGTGG
5000
GCATTGATCC GTTTCGTCTG CTGCAGAACA GCCAGGTGTA
TAGCCTGATT
5050
CGTCCGAACG AAAACCCGGC GCATAAAAGC CAGCTGGTGT
GGATGGCGTG
5100
CCATAGCGCG GCGTTTGAAG ACCTGCGTGT GAGCAGCTTT
ATTCGTGGCA
5150
CCAAAGTGGT GCCGCGTGGC AAACTGAGCA CCCGTGGCGT
GCAGATTGCG
5200
AGCAACGAAA ACATGGAAAC GATGGAAAGC AGCACCCTGG
AACTGCGTAG
5250
CCGTTATTGG GCGATTCGTA CCCGTAGCGG CGGCAACACC
AACCAGCAGC
5300
GTGCGAGCAG CGGCCAGATT AGCATTCAGC CGACCTTTAG
CGTGCAGCGT
5350
AACCTGCCGT TTGATCGTCC GACCATTATG GCGGCGTTTA
CCGGCAACAC
5400
CGAAGGCCGT ACCAGCGATA TGCGTACCGA AATTATTCGT
CTGATGGAAA
5450
GCGCGCGTCC GGAAGATGTG AGCTTTCAGG GCCGTGGCGT
GTTTGAACTG
5500
AGCGATGAAA AAGCGACCAG CCCGATTGTG CCGAGCTTTG
ATATGAGCAA
5550
CGAAGGCAGC TACTTTTTCG GCGATAACGC GGAAGAATAT
GATAACTAAG
5600
ACCCATGGGA ATTCGCAATT CCCGGGGATC CGTCGACCTG
CAGCCAAGCT
5650
CCCAAGCTTG GCTGTTTTGG CGGATGAGAG AAGATTTTCA
GCCTGATACA
5700
GATTAAATCA GAACGCAGAA GCGGTCTGAT AAAACAGAAT
TTGCCTGGCG
5750
GCAGTAGCGC GGTGGTCCCA CCTGACCCCA TGCCGAACTC
AGAAGTGAAA
5800
CGCCGTAGCG CCGATGGTAG TGTGGGGTCT CCCCATGCGA
GAGTAGGGAA
5850
CTGCCAGGCA TCAAATAAAA CGAAAGGCTC AGTCGAAAGA
CTGGGCCTTT
5900
CGTTTTATCT GTTGTTTGTC GGTGAACGCT CTCCTGAGTA
GGACAAATCC
5950
GCCGGGAGCG GATTTGAACG TTGCGAAGCA ACGGCCCGGA
GGGTGGCGGG
6000
CAGGACGCCC GCCATAAACT GCCAGGCATC AAATTAAGCA
GAAGGCCATC
6050
CTGACGGATG GCCTTTTTGC GTTTCTACAA ACTCTTTTGT
TTATTTTTCT
6100
AAATACATTC AAATATGTAT CCGCTCATGA GACAATAACC
CTGATAAATG
6150
CTTCAATAAT GGAAGATCTT CCAACATCAC AGGTAAACAG
AAACGTCGGG
6200
TCGATCGGGA AATTCTTTCC CGGACGGCGC GGGGTTGGGC
AAGCCGCAGG
6250
CGCGTCAGTG CTTTTAGCGG GTGTCGGGGC GCAGCCATGA
CCCAGTCACG
6300
TAGCGATAGC GGAGTGTATA CTGGCTTAAC TATGCGGCAT
CAGAGCAGAT
6350
TGTACTGAGA GTGCACCATA TGCGGTGTGA AATACCGCAC
AGATGCGTAA
6400
GGAGAAAATA CCGCATCAGG CGCTCTTCCG CTTCCTCGCT
CACTGACTCG
6450
CTGCGCTCGG TCGTTCGGCT GCGGCGAGCG GTATCAGCTC
ACTCAAAGGC
6500
GGTAATACGG TTATCCACAG AATCAGGGGA TAACGCAGGA
AAGAACATGT
6550
GAGCAAAAGG CCAGCAAAAG GCCAGGAACC GTAAAAAGGC
CGCGTTGCTG
6600
GCGTTTTTCC ATAGGCTCCG CCCCCCTGAC GAGCATCACA
AAAATCGACG
6650
CTCAAGTCAG AGGTGGCGAA ACCCGACAGG ACTATAAAGA
TACCAGGCGT
6700
TTCCCCCTGG AAGCTCCCTC GTGCGCTCTC CTGTTCCGAC
CCTGCCGCTT
6750
ACCGGATACC TGTCCGCCTT TCTCCCTTCG GGAAGCGTGG
CGCTTTCTCA
6800
TAGCTCACGC TGTAGGTATC TCAGTTCGGT GTAGGTCGTT
CGCTCCAAGC
6850
TGGGCTGTGT GCACGAACCC CCCGTTCAGC CCGACCGCTG
CGCCTTATCC
6900
GGTAACTATC GTCTTGAGTC CAACCCGGTA AGACACGACT
TATCGCCACT
6950
GGCAGCAGCC ACTGGTAACA GGATTAGCAG AGCGAGGTAT
GTAGGCGGTG
7000
CTACAGAGTT CTTGAAGTGG TGGCCTAACT ACGGCTACAC
TAGAAGGACA
7050
GTATTTGGTA TCTGCGCTCT GCTGAAGCCA GTTACCTTCG
GAAAAAGAGT
7100
TGGTAGCTCT TGATCCGGCA AACAAACCAC CGCTGGTAGC
GGTGGTTTTT
7150
TTGTTTGCAA GCAGCAGATT ACGCGCAGAA AAAAAGGATC
TCAAGAAGAT
7200
CCTTTGATCT TTTCTACGGG GTCTGACGCT CAGTGGAACG
AAAACTCACG
7250
TTAAGGGATT TTGGTCATGA GATTATCAAA AAGGATCTTC
ACCTAGATCC
7300
TTTTAAATTA AAAATGAAGT TTTAAATCAA TCTAAAGTAT
ATATGAGTAA ACTTGGTCTG ACAGTCTAGA
```

TABLE 7
DNA sequence of plasmid pYA5126.

```
Range: 1 to 7392
    50
AGATCTAGCC CGCCTAATGA GCGGGCTTTT TTTTAATTCG
CAATTCCCCG
  100
ATGCATAATG TGCCTGTCAA ATGGACGAAG CAGGGATTCT
GCAAACCCTA
  150
TGCTACTCCG TCAAGCCGTC AATTGTCTGA TTCGTTACCA
ATTATGACAA
  200
CTTGACGGCT ACATCATTCA CTTTTTCTTC ACAACCGGCA
CGGAACTCGC
  250
TCGGGCTGGC CCCGGTGCAT TTTTTAAATA CCCGCGAGAA
ATAGAGTTGA
  300
TCGTCAAAAC CAACATTGCG ACCGACGGTG GCGATAGGCA
TCCGGGTGGT
  350
GCTCAAAAGC AGCTTCGCCT GGCTGATACG TTGGTCCTCG
CGCCAGCTTA
  400
AGACGCTAAT CCCTAACTGC TGGCGGAAAA GATGTGACAG
ACGCGACGGC
  450
GACAAGCAAA CATGCTGTGC GACGCTGGCG ATATCAAAAT
TGCTGTCTGC
  500
CAGGTGATCG CTGATGTACT GACAAGCCTC GCGTACCCGA
TTATCCATCG
  550
GTGGATGGAG CGACTCGTTA ATCGCTTCCA TGCGCCGCAG
TAACAATTGC
  600
TCAAGCAGAT TTATCGCCAG CAGCTCCGAA TAGCGCCCTT
CCCCTTGCCC
  650
GGCGTTAATG ATTTGCCCAA ACAGGTCGCT GAAATGCGGC
TGGTGCGCTT
  700
CATCCGGGCG AAAGAACCCC GTATTGGCAA ATATTGACGG
CCAGTTAAGC
  750
CATTCATGCC AGTAGGCGCG CGGACGAAAG TAAACCCACT
GGTGATACCA
  800
TTCGCGAGCC TCCGGATGAC GACCGTAGTG ATGAATCTCT
CCTGGCGGGA
  850
ACAGCAAAAT ATCACCCGGT CGGCAAACAA ATTCTCGTCC
CTGATTTTTC
  900
ACCACCCCCT GACCGCGAAT GGTGAGATTG AGAATATAAC
CTTTCATTCC
  950
CAGCGGTCGG TCGATAAAAA AATCGAGATA ACCGTTGGCC
TCAATCGGCG
1000
TTAAACCCGC CACCAGATGG GCATTAAACG AGTATCCCGG
CAGCAGGGGA
1050
TCATTTTGCG CTTCAGCCAT ACTTTTCATA CTCCCGCCAT
TCAGAGAAGA
1100
AACCAATTGT CCATATTGCA TCAGACATTG CCGTCACTGC
GTCTTTTACT
1150
GGCTCTTCTC GCTAACCAAA CCGGTAACCC CGCTTATTAA
AAGCATTCTG
1200
TAACAAAGCG GGACCAAAGC CATGACAAAA ACGCGTAACA
AAAGTGTCTA
1250
TAATCACGGC AGAAAAGTCC ACATTGATTA TTTGCACGGC
GTCACACTTT
1300
GCTATGCCAT AGCATTTTTA TCCATAAGAT TAGCGGATCC
TACCTGACGC
1350
TTTTTATCGC AACTCTCTAC TGTTTCTCCA TACCCGTTTT
TTTGGGCTAG
1400
CGAATTCTGA GAACAAACTA AATGGATAAA TTTCGTGTTC
AGGGGCCAAC
1450
GAAGCTCCAG GGCGAAGTCA CAATTTCCGG CGCTAAAAAT
GCTGCTCTGC
1500
CTATCCTTTT TGCCGCACTA CTGGCGGAAG AACCGGTAGA
GATCCAGAAC
1550
GTCCCGAAAC TGAAAGACGT CGATACATCA ATGAAGCTGC
TAAGCCAGCT
1600
GGGTGCGAAA GTAGAACGTA ATGGTTCTGT GCATATTGAT
GCCCGCGACG
1650
TTAATGTATT CTGCGCACCT TACGATCTGG TTAAAACCAT
GCGTGCTTCT
1700
ATCTGGGCGC TGGGGCCGCT GGTAGCGCGC TTTGGTCAGG
GGCAAGTTTC
1750
ACTACCTGGC GGTTGTACGA TCGGTGCGCG TCCGGTTGAT
CTACACATTT
1800
CTGGCCTCGA ACAATTAGGC GCGACCATCA AACTGGAAGA
AGGTTACGTT
1850
AAAGCTTCCG TCGATGGTCG TTTGAAAGGT GCACATATCG
TGATGGATAA
1900
AGTCAGCGTT GGCGCAACGG TGACCATCAT GTGTGCTGCA
ACCCTGGCGG
1950
AAGGCACCAC GATTATTGAA AACGCAGCGC GTGAACCGGA
AATCGTCGAT
2000
ACCGCGAACT TCCTGATTAC GCTGGGTGCG AAAATTAGCG
GTCAGGGCAC
2050
CGATCGTATC GTCATCGAAG GTGTGGAACG TTTAGGCGGC
GGTGTCTATC
2100
GCGTTCTGCC GGATCGTATC GAAACCGGTA CTTTCCTGGT
GGCGGCGGCG
2150
ATTTCTCGCG GCAAAATTAT CTGCCGTAAC GCGCAGCCAG
ATACTCTCGA
2200
CGCCGTGCTG GCGAAACTGC GTGACGCTGG AGCGGACATC
GAAGTCGGCG
2250
AAGACTGGAT TAGCCTGGAT ATGCATGGCA AACGTCCGAA
GGCTGTTAAC
2300
GTACGTACCG CGCCGCATCC GGCATTCCCG ACCGATATGC
AGGCCCAGTT
2350
CACGCTGTTG AACCTGGTGG CAGAAGGGAC CGGGTTTATC
ACCGAAACGG
2400
TCTTTGAAAA CCGCTTTATG CATGTGCCAG AGCTGAGCCG
TATGGGCGCG
2450
CACGCCGAAA TCGAAAGCAA TACCGTTATT TGTCACGGTG
TTGAAAAACT
2500
TTCTGGCGCA CAGGTTATGG CAACCGATCT GCGTGCATCA
GCAAGCCTGG
2550
TGCTGGCTGG CTGTATTGCG GAAGGGACGA CGGTGGTTGA
TCGTATTTAT
2600
CACATCGATC GTGGCTACGA ACGCATTGAA GACAAACTGC
GCGCTTTAGG
2650
TGCAAATATT GAGCGTGTGA AAGGCGAATA AGAATTCAGG
AAAAAAACGC
2700
TGTGAAAAAT GTTGGTTTTA TCGGCTGGCG CGGAATGGTC
GGCTCTGTTC
2750
TCATGCAACG CATGGTAGAG GAGCGCGATT TCGACGCTAT
TCGCCCTGTT
2800
TTCTTTTCTA CCTCCCAGTT TGGACAGGCG GCGCCCACCT
TCGGCGACAC
2850
CTCCACCGGC ACGCTACAGG ACGCTTTTGA TCTGGATGCG
CTAAAAGCGC
2900
TCGATATCAT CGTGACCTGC CAGGGCGGCG ATTATACCAA
CGAAATTTAT
2950
CCAAAGCTGC GCGAAAGCGG ATGGCAGGGT TACTGGATTG
ATGCGGCTTC
3000
TACGCTGCGC ATGAAAGATG ATGCCATTAT TATTCTCGAC
CCGGTCAACC
3050
AGGACGTGAT TACCGACGGC CTGAACAATG GCGTGAAGAC
CTTTGTGGGC
3100
GGTAACTGTA CCGTTAGCCT GATGTTGATG TCGCTGGGCG
GTCTCTTTGC
3150
CCATAATCTC GTTGACTGGG TATCCGTCGC GACCTATCAG
GCCGCCTCCG
3200
GCGGCGGCGC GCGCCATATG CGCGAGCTGT TAACCCAGAT
GGGTCAGTTG
3250
TATGGCCATG TCGCCGATGA ACTGGCGACG CCGTCTTCCG
CAATTCTTGA
3300
TATTGAACGC AAAGTTACGG CATTGACCCG CAGCGGCGAG
CTGCCGGTTG
3350
ATAACTTTGG CGTACCGCTG GCGGGAAGCC TGATCCCCTG
GATCGACAAA
3400
CAGCTCGATA ACGGCCAGAG CCGCGAAGAG TGGAAAGGCC
AGGCGGAAAC
3450
CAACAAGATT CTCAATACTG CCTCTGTGAT TCCGGTTGAT
GGTTTGTGTG
3500
TGCGCGTCGG CGCGCTGCGC TGTCACAGCC AGGCGTTCAC
CATCAAGCTG
3550
AAAAAAGAGG TATCCATTCC GACGGTGGAA GAACTGCTGG
CGGCACATAA
3600
TCCGTGGGCG AAAGTGGTGC CGAACGATCG TGATATCACT
ATGCGCGAAT
3650
TAACCCCGGC GGCGGTGACC GGCACGTTGA CTACGCCGGT
TGGTCGTCTG
3700
CGTAAGCTGA ACATGGGGCC AGAGTTCTTG TCGGCGTTTA
CCGTAGGCGA
3750
CCAGTTGTTA TGGGGCGCCG CCGAGCCGCT GCGTCGAATG
CTGCGCCAGT
3800
TGGCGTAGTC TAGCTGCACG ATACCGTCGA CTTGTACATA
GACTCGCTCC
3850
GAAATTAAAG AACACTTAAA TTATCTACTA AAGGAATCTT
TAGTCAAGTT
3900
TATTTAAGAT GACTTAACTA TGAATACACA ATTGATGGGT
GAGCGTAGGA
3950
GCATGCTTAT GCGAAAGGCC ATCCTGACGG ATGGCCTTTT
TGGATCTTCC
4000
GGAAGACCTT CCATTCTGAA ATGAGCTGTT GACAATTAAT
CATCCGGCTC
4050
GTATAATGTG TGGAATTGTG AGCGGATAAC AATTTCACAC
AGGAAACAGA
4100
CCATGGCGAC CAAAGGCACC AAACGTAGCT ATGAACAGAT
GGAAACCGAT
4150
GGCGAACGTC AGAACGCGAC CGAAATTCGT GCGAGCGTGG
GCAAAATGAT
4200
TGATGGCATT GGCCGTTTTT ATATTCAGAT GTGCACCGAA
CTGAAACTGA
4250
GCGATTATGA AGGCCGTCTG ATTCAGAACA GCCTGACCAT
TGAACGTATG
4300
GTGCTGAGCG CGTTTGATGA ACGTCGTAAC AAATATCTGG
AAGAACATCC
4350
GAGCGCGGGC AAAGATCCAA AGAAAACCGG CGGCCCGATT
TATCGTCGTG
4400
TGGATGGCAA ATGGCGTCGT GAACTGATTC TGTATGATAA
AGAAGAAATT
4450
CGTCGTATTT GGCGTCAGGC GAACAACGGC GATGATGCGA
CCGCGGGCCT
4500
GACCCACATG ATGATTTGGC ATAGCAACCT GAACGATGCG
ACCTATCAGC
4550
GTACCCGTGC GCTGGTGCGT ACCGGCATGG ACCCACGTAT
GTGCAGCCTG
4600
ATGCAGGGCA GCACCCTGCC GCGTCGTAGC GGTGCAGCAG
GTGCAGCAGT
4650
GAAAGGCGTG GGTACGATGG TGATGGAACT GATTCGTATG
ATTAAACGTG
4700
GCATTAACGA TCGTAACTTT TGGCGTGGCG AAAACGGCCG
TCGTACCCGT
4750
ATTGCGTATG AACGTATGTG CAACATTCTG AAAGGCAAAT
TTCAGACCGC
4800
GGCGCAGCGT ACGATGGTGG ATCAAGTGCG TGAAAGCCGT
AACCCGGGCA
4850
ACGCGGAATT TGAAGACCTG ATTTTTCTGG CGCGTAGCGC
GCTGATTCTG
4900
CGTGGCAGCG TGGCGCATAA AAGCTGCCTG CCGGCGTGCG
TGTATGGCCC
4950
GGCGGTGGCG AGCGGCTATG ATTTTGAACG TGAAGGCTAT
AGCCTGGTGG
5000
GCATTGATCC GTTTCGTCTG CTGCAGAACA GCCAGGTGTA
TAGCCTGATT
5050
CGTCCGAACG AAAACCCGGC GCATAAAAGC CAGCTGGTGT
GGATGGCGTG
5100
CCATAGCGCG GCGTTTGAAG ACCTGCGTGT GAGCAGCTTT
ATTCGTGGCA
5150
CCAAAGTGGT GCCGCGTGGC AAACTGAGCA CCCGTGGCGT
GCAGATTGCG
5200
AGCAACGAAA ACATGGAAAC GATGGAAAGC AGCACCCTGG
AACTGCGTAG
5250
CCGTTATTGG GCGATTCGTA CCCGTAGCGG CGGCAACACC
AACCAGCAGC
5300
GTGCGAGCAG CGGCCAGATT AGCATTCAGC CGACCTTTAG
CGTGCAGCGT
5350
AACCTGCCGT TTGATCGTCC GACCATTATG GCGGCGTTTA
CCGGCAACAC
5400
CGAAGGCCGT ACCAGCGATA TGCGTACCGA AATTATTCGT
CTGATGGAAA
5450
GCGCGCGTCC GGAAGATGTG AGCTTTCAGG GCCGTGGCGT
GTTTGAACTG
5500
AGCGATGAAA AAGCGACCAG CCCGATTGTG CCGAGCTTTG
ATATGAGCAA
5550
CGAAGGCAGC TACTTTTTCG GCGATAACGC GGAAGAATAT
GATAACACAT
5600
ATGTTTCTGT TGGTACCTCT ACACTGGCGG CGTATCGTAC
ACTGGATTTC
5650
CATGATTCTA ACGTTAAATA AGACCCATGG GAATTCGCAA
TTCCCGGGGA
5700
TCCGTCGACC TGCAGCCAAG CTCCCAAGCT TGGCTGTTTT
GGCGGATGAG
5750
AGAAGATTTT CAGCCTGATA CAGATTAAAT CAGAACGCAG
AAGCGGTCTG
5800
ATAAAACAGA ATTTGCCTGG CGGCAGTAGC GCGGTGGTCC
CACCTGACCC
5850
CATGCCGAAC TCAGAAGTGA AACGCCGTAG CGCCGATGGT
AGTGTGGGGT
5900
CTCCCCATGC GAGAGTAGGG AACTGCCAGG CATCAAATAA
AACGAAAGGC
5950
TCAGTCGAAA GACTGGGCCT TTCGTTTTAT CTGTTGTTTG
TCGGTGAACG
6000
CTCTCCTGAG TAGGACAAAT CCGCCGGGAG CGGATTTGAA
CGTTGCGAAG
6050
CAACGGCCCG GAGGGTGGCG GGCAGGACGC CCGCCATAAA
CTGCCAGGCA
6100
TCAAATTAAG CAGAAGGCCA TCCTGACGGA TGGCCTTTTT
GCGTTTCTAC
6150
AAACTCTTTT GTTTATTTTT CTAAATACAT TCAAATATGT
ATCCGCTCAT
6200
GAGACAATAA CCCTGATAAA TGCTTCAATA ATGGAAGATC
TTCCAACATC
6250
ACAGGTAAAC AGAAACGTCG GGTCGATCGG GAAATTCTTT
CCCGGACGGC
6300
GCGGGGTTGG GCAAGCCGCA GGCGCGTCAG TGCTTTTAGC
GGGTGTCGGG
6350
GCGCAGCCAT GACCCAGTCA CGTAGCGATA GCGGAGTGTA
TACTGGCTTA
6400
ACTATGCGGC ATCAGAGCAG ATTGTACTGA GAGTGCACCA
TATGCGGTGT
6450
GAAATACCGC ACAGATGCGT AAGGAGAAAA TACCGCATCA
GGCGCTCTTC
6500
CGCTTCCTCG CTCACTGACT CGCTGCGCTC GGTCGTTCGG
CTGCGGCGAG
6550
CGGTATCAGC TCACTCAAAG GCGGTAATAC GGTTATCCAC
AGAATCAGGG
6600
GATAACGCAG GAAAGAACAT GTGAGCAAAA GGCCAGCAAA
AGGCCAGGAA
6650
CCGTAAAAAG GCCGCGTTGC TGGCGTTTTT CCATAGGCTC
CGCCCCCCTG
6700
ACGAGCATCA CAAAAATCGA CGCTCAAGTC AGAGGTGGCG
AAACCCGACA
6750
GGACTATAAA GATACCAGGC GTTTCCCCCT GGAAGCTCCC
TCGTGCGCTC
6800
TCCTGTTCCG ACCCTGCCGC TTACCGGATA CCTGTCCGCC
TTTCTCCCTT
6850
CGGGAAGCGT GGCGCTTTCT CATAGCTCAC GCTGTAGGTA
TCTCAGTTCG
6900
GTGTAGGTCG TTCGCTCCAA GCTGGGCTGT GTGCACGAAC
CCCCCGTTCA
6950
GCCCGACCGC TGCGCCTTAT CCGGTAACTA TCGTCTTGAG
TCCAACCCGG
7000
TAAGACACGA CTTATCGCCA CTGGCAGCAG CCACTGGTAA
CAGGATTAGC
7050
AGAGCGAGGT ATGTAGGCGG TGCTACAGAG TTCTTGAAGT
GGTGGCCTAA
7100
CTACGGCTAC ACTAGAAGGA CAGTATTTGG TATCTGCGCT
CTGCTGAAGC
7150
CAGTTACCTT CGGAAAAAGA GTTGGTAGCT CTTGATCCGG
CAAACAAACC
7200
ACCGCTGGTA GCGGTGGTTT TTTTGTTTGC AAGCAGCAGA
TTACGCGCAG
7250
AAAAAAAGGA TCTCAAGAAG ATCCTTTGAT CTTTTCTACG
GGGTCTGACG
7300
CTCAGTGGAA CGAAAACTCA CGTTAAGGGA TTTTGGTCAT
GAGATTATCA
7350
AAAAGGATCT TCACCTAGAT CCTTTTAAAT TAAAAATGAA
GTTTTAAATC AATCTAAAGT ATATATGAGT AAACTTGGTC
TGACAGTCTA GA
```

### References for Example 6

49. Ashraf S, Kong W, Wang S, Yang J, & Curtiss R, III (2011). Protective cellular responses elicited by vaccination with influenza nucleoprotein delivered by a live recombinant attenuated Salmonella vaccine. Vaccine 29(23):3990-4002.
50. Velders M P, et al. (2001). Defined flanking spacers and enhanced proteolysis is essential for eradication of established tumors by an epitope string DNA vaccine. J Immunol 166(9):5366-5373.
51. Bakshi C S, et al. (2000). Identification of SopE2, a Salmonella secreted protein which is highly homologous to SopE and involved in bacterial invasion of epithelial cells. J Bacteriol 182(8):2341-2344.
52. Kubori T & Galan J E (2003). Temporal regulation of Salmonella virulence effector function by proteasome-dependent protein degradation. Cell 115(3):333-342.
53. Joly E & Hudrisier D (2003). What is trogocytosis and what is its purpose? Nat Immunol 4(9):815.
54. Daubeuf S, Puaux A L, Joly E, & Hudrisier D (2006). A simple trogocytosis-based method to detect, quantify, characterize and purify antigen-specific live lymphocytes by flow cytometry, via their capture of membrane fragments from antigen-presenting cells. Nat Protoc 1(6):2536-2542.

### Introduction to Example 7

Tuberculosis (TB) is a disease of antiquity and of the present. Approximately 8 million individuals are diagnosed with TB annually throughout the world. Although not all individuals who are infected develop active disease, among those who do, nearly two million die each year. There are effective antibiotics to treat active disease, but the treatment regimen is long (at least 6 months), leading to significant non-compliance with treatment. Each year, there are increasing numbers of TB cases caused by strains of Mycobacterium tuberculosis (the causative agent of TB) that are resistant to many (Multi-drug resistant or MDR) or essentially all (Extremely Drug Resistant or XDR) of the available antibiotics, thereby severely compromising physicians' abilities to cure the disease. There is an available vaccine, the attenuated strain of Mycobacterium bovis, M. bovis BCG (Bacille Calmette Guérin), which significantly reduces the complications of TB (meningitis, miliary TB) in infants and young children. However, the BCG vaccine does not confer long-lasting protection on immunized individuals who become susceptible to infection as adolescents, young adults and elderly adults.

Resistance to TB is a consequence of the development of a cell-mediated immune response which both controls and contains the infection (55, 65). CD4⁺ T cells are the master regulators of the protective immune response, initially in the role of effector cells, but also giving rise to memory T cells (65). CD8⁺ T cells are also crucial to the development of a robust protective response, through their production of the potent cytokine, Interferon γ, and their cytolytic functions (55, 65). CD8⁺ T cells also give rise to memory cells that are crucial for long-term control of infection (65). The contributions of both kinds of T cells for protective immunity were initially shown in animal studies, by passive transfer of each kind of T cell subset (64, 64) and by studies using mice that were deficient in either CD4⁺ or CD8⁺ T cells (56, 57). Mice that lacked either CD4⁺ or CD8⁺ T cells were highly susceptible to infection with M. tuberculosis and were unable to control the infections. The correlations of the animal studies with humans can be observed in individuals infected with the Human Immunodeficiency Virus (HIV), who have decreasing numbers of CD4⁺ T cells as their disease progresses and who become increasingly susceptible to M. tuberculosis infection as a result (60). Similarly, individuals who are genetically deficient for CD8⁺ T cell production are also more susceptible to TB than individuals with intact cytokine-mediated macrophage activation pathways (66). Thus, development of an effective vaccine against M. tuberculosis, which confers long-lasting protection, must elicit a robust CMI response that generates effector CD4⁺ T cells, memory CD4⁺ T cells, effector CD8⁺ T cells and memory CD8⁺ T cells.

### Materials and Methods for Example 7.

### Bacterial Strains and Plasmids.

The bacterial strains and plasmids used in these studies are listed in Table 8. Serovar Typhimurium strains are derived from the highly virulent strain UK-1. Bacteriophage P22HTint was used for generalized transduction. E. coli and serovar Typhimurium cultures were grown in LB broth or on LB agar plates at 37° C. Diaminopimelic acid (DAP) was added at the concentration of 50 μg/ml for the growth of Asd⁻ strains [19] and in case of regulated delayed lysis vector LB was supplemented with 0.2% arabinose.

TABLE 8
Bacterial strains and plasmids used in this study.

| A. Strain | | |
|---|---|---|
| S. enterica | | |
| Serovar | | Source or |
| Typhimurium | Genotype | Reference |
| χ11021 | ΔasdA27::TT araC | Same as χ11017 |
| | P_{BAD}c2Δ(araC P_{BAD})- | described by Ashraf |
| | 5::P22 P_{R} araBAD Δ(gmd- | et al., (2011) |
| | fcl)-26 Δpmi- | |
| | 2426ΔrelA198:: araC P_{BAD} | |
| | lacITT ΔP_{murA25}::TT araC | |
| | P_{BAD} murA | |
| χ11246 | ΔasdA27::TT araC P_{BAD} | χ11017 = χ11021 |
| | c2Δ(araC P_{BAD})-5::P22 P_{R} | |
| | araBAD Δ(gmd-fcl)-26 | |
| | Δpmi-2426ΔrelA198::araC | |
| | P_{BAD} lacITT ΔP_{murA25}::TT | |
| | araC P_{BAD} murA ΔsifA | |
| χ11324 | ΔP_{murA25}::TT araC P_{BAD} | This study |
| | murA ΔasdA27::TT araC | |
| | P_{BAD} c2 Δ(araC P_{BAD})- | |
| | 5::P22 P_{R} araBAD Δ(gmd- | |
| | fcl)-26 Δpmi-2426 | |
| | ΔrelA198::araC P_{BAD} lacI | |
| | TT ΔtlpA181 ΔsseL ΔP_{hilA}:: | |
| | P_{trc ΔlacO888} | |
| χ11327 | ΔP_{murA25}::TT araC P_{BAD} | This study |
| | murA ΔasdA27::TT araC | |
| | P_{BAD} c2 Δ(araC P_{BAD})- | |
| | 5::P22 P_{R} araBAD Δ(gmd- | |
| | fcl)-26 Δpmi-2426 | |
| | ΔrelA198::araC P_{BAD} lacI | |
| | TT ΔtlpA181 ΔsseL ΔP_{hilA}:: | |
| | P_{trc ΔlacO888} ΔsifA26 | |
| χ11412 | ΔasdA27::TT araC P_{BAD} c2 | This study |
| | ΔP_{murA25}::TT araC P_{BAD} | |
| | murA Δ(araC P_{BAD})-5::P22 | |
| | P_{R} araBAD Δ(gmd-fcl)-26 | |
| | ΔrelA198::araC P_{BAD} lacI | |
| | TT Δpmi-2426 ΔtlpA181 | |
| | ΔsseL116 ΔP_{hilA}::P_{trc ΔlacO888} | |
| | hilA ΔsifA26 ΔaraE25 | |
| | ΔendA2311 | |

| | | Source or |
|---|---|---|
| Plasmids | Relevant Characteristics | Reference |
| pYA3681 | Lysis vector P_{trc} promoter | 2 |
| pYA3816 | DNA delivery vector, pUC | FIG. 29 |
| | ori, P_{CMV} promoter, Kozak | |
| | sequence-ppe18-bovine | |
| | growth hormone (BGH) | |
| | polyA | |
| pYA4683 | Asd⁺ MurA⁺ Lysis vector | FIG. 27 |
| | with p15A ori, P_{trc} | |
| | promoter, sopE2_{Nt}-ppe18 | |
| pYA4851 | Asd⁺ MurA⁺ Lysis vector | FIG. 26 |
| | with pBR ori, P_{trc} promoter, | |
| | sopE2_{Nt}-ppe18 (derived | |
| | from pYA3681) | |
| pYA4856 | Asd⁺ MurA⁺ Lysis vector | FIG. 28 |
| | with pBR ori, P_{trc} promoter, | |
| | ppe18 (derived from | |
| | pYA3681) | |
| pYA4890 | Asd⁺ MurA⁺ Lysis vector | FIG. 23, 3 |
| | with pBR ori, P_{trc} promoter, | |
| | sopE_{Nt}-esxA-esxA-esxB | |
| | (SD) bla_{SS}-fbpA-bla_{CT} | |
| pYA4891 | Asd⁺ MurA⁺ Lysis vector | FIG. 24, 3 |
| | with p15A ori, P_{trc} | |
| | promoter, sopE_{Nt}-esxA- | |
| | esxA-esxe (SD) bla_{SS}- | |
| | fbpA-bla_{CT} | |
| pYA4893 | Asd⁺ MurA⁺ Lysis vector | FIG. 25, 3 |
| | with pBR ori, P_{trc} promoter, | |
| | ompCₛₛ-esxA-esxA-esxB | |
| | (SD) bla_{SS}-fbpA-bla_{CT} | |

1 Ashraf, S., W. Kong, S. Wang, J. Yang and R. Curtiss III. Protective cellular responses elicited by vaccination with influenza nucleoprotein delivered by a live recombinant Salmonella vaccine. Vaccine 2011, pp. 3990-4002, Vol. 29.
2 Kong, W. S. Y. Wanda, X. Zhang, W. Bollen, S. A. Tinge, K. L. Roland, and R. Curtiss, 3rd. Regulated programmed lysis of recombinant Salmonella in host tissues to release protective antigens and confer biological containment. Proc Natl Acad Sci USA, 2008, pp. 9361-9366, Vol. 105.
3 Juárez-Rodríguez, M. D., J. Yang, R. Keder, P. Alamuri, R. Curtiss III and J. E. Clark-Curtiss. Live attenuated Salmonella vaccines displaying regulated delayed lysis and delayed antigen synthesis to confer protection against Mycobacterium tuberculosis. Infect. Immun. 2012, pp. 815-831, Vol. 80.

### Strain Construction and Characterization.

ΔtlpA181 is a defined in-frame deletion of tlpA in χ3761 (Salmonella Typhimurium UK-1). It was introduced into χ11017 by P22 transduction to yield strain χ11226.

ΔsseL116 is a defined in-frame deletion of sseL in χ3761 (Salmonella Typhimurium UK-1). It was introduced into χ11226 by transduction using a P22 lysate to transduce ΔsseL116 to yield strain χ11228.

ΔP_{hilA}:: P_{trc ΔlacO888} hilA is a deletion of the promoter of hilA gene and replacement with P_{trc ΔlacO888} to result in regulated expression of hilA. It was introduced into χ11228 by P22 transduction to yield χ11234.

### General DNA Procedures and Plasmid Stability.

These procedures are as described above.

M. tuberculosis Antigens

The M. tuberculosis antigens used in the studies and the genes that encode them are ESAT-6, encoded by esxA; CFP-10, encoded by esxB; Antigen 85A (Ag85A), encoded by fbpA and Mtb39A, encoded by ppe-18. All of these antigens are known to contain T cell epitopes (57, 58, 62, 67, 68) and have been shown to elicit protection to challenge with aerosolized M. tuberculosis in mice and guinea pigs (57, 58, 62, 67). These antigens have been incorporated into candidate vaccines to protect against infection of humans.

Codon Optimization of fbpA Gene.

A DNA fragment containing the nucleotide sequence of the fbpA gene (Rv3804c) encoding the Ag85A protein was PCR-amplified from M. tuberculosis H37Rv chromosomal DNA. The PCR product of 1111-bp was digested with XbaI and EcoRI and cloned into XbaI-EcoRI-digested pBK-CMV (Stratagene) to generate pYA3817. To optimize the expression of fbpA in Salmonella, pYA3817 was used as the template to substitute twenty-four codons of fbpA with the most frequently found codons in Salmonella using a Quick-Change site-directed mutagenesis kit (Stratagene) with appropriate primers. The fbpA codons 62 (TCC to TCT), 63 (CGG to CGT), 66 (TTG to CTG), 88 (AGT to AGC), 94 (CCC to CCG), 136 (TCA to TCT), 145 (CCC to CCG), 173 (AGG to CGT), 177 (CCC to CCG), 179 (GGA to GGT), 200 (CCC to CCG), 207 (GGA to GGT), 213 (TTG to CTG), 215(CCC to CCG), 221 (CCC to CCG), 255 (TTG to CTG), 258 (GGG to GGT), 294(CGG to CGT), 336 (CCC to CCG), 240 (CGG to CGT), 346 (CCC to CCG), 349 (GGG to GGT), 250 (CCC to CCG) and 252 (CCC to CCG) were substituted. The resulting recombinant plasmid containing all of the optimized sequences of fbpA was named pYA3932 (61).

### Vector Construction.

The primer pairs used to construct the plasmids used in this study are listed in Table 9. Vent DNA polymerase was used for the PCR reactions with dNTPs (Invitrogen).

TABLE 9
Primer Pair Sequences

| | |
|---|---|
| Primer | Sequence (5′→3′) |

Cloning of fbpA (Rv3804c)

| | |
|---|---|
| 85A-F1c | GCCGGGTCTAGAGCCTGCAGTCTG |
| 85A-R1 | CTAGATGTTGTGAATTCTCGGAGCTAGGCGCCCT |

Optimization of fbpA

| | |
|---|---|
| 85A-F1 | CCGCGGGGGCATTTTCTCGTCCGGGCCTGCCGGTGGAGTAC |
| 85A-R1B | GTACTCCACCGGCAGGCCCGGACGAGAAAATGCCCCCGCGG |
| 85A-F2 | CAATTCCAAAGCGGTGGTGCCAACTCGCCGGCCCTGTACCTGC |
| 85A-R2 | GCAGGTACAGGGCCGGCGAGTTGGCACCACCGCTTTGGAATTG |
| 85A-F3 | GTCTAGCTTCTACTCCGACTGGTACCAGCCGGCCTGCGGCAAG |
| 85A-R3 | CTTGCCGCAGGCCGGCTGGTACCAGTCGGAGTAGAAGCTAGAC |
| 85A-F4 | CTGCAGGCCAACCGTCACGTCAAGCCGACCGGTAGCGCCGTCG |
| 85A-R4 | CGACGGCGCTACCGGTCGGCTTGACGTGACGGTTGGCCTGCAG |
| 85A-F5 | CGCTGGCGATCTATCACCCGCAGCAGTTCGTCTACGCG |
| 85A-R5 | CGCGTAGACGAACTGCTGCGGGTGATAGATCGCCAGCG |
| 85A-F6 | CTACGCGGGTGCGATGTCGGGCCTGCTGGACCCGTCCCAGGCG |
| 85A-R6 | CGCCTGGGACGGGTCCAGCAGGCCCGACATCGCACCCGCGTAG |
| 85A-F7 | GCTGGACCCGTCCCAGGCGATGGGTCCGACCCTGATCGGCCTG |
| 85A-R7 | CAGGCCGATCAGGGTCGGACCCATCGCCTGGGACGGGTCCAGC |
| 85A-F8 | CGCAACGACCCGCTGCTGAACGTCGGTAAGCTGATCGCCAAC |
| 85A-R8 | GTTGGCGATCAGCTTACCGACGTTCAGCAGCGGGTCGTTGCG |
| 85A-F9 | CAAGTTCCTCGAGGGCTTCGTGCGTACCAGCAACATCAAGTTC |
| 85A-R9 | GAACTTGATGTTGCTGGTACGCACGAAGCCCTCGAGGAACTTG |
| 85A-F10 | CAACGCTATGAAGCCGGACCTGCAACGTGCACTGGGTGCCAC |
| 85A-R10 | GTGGCACCCAGTGCACGTTGCAGGTCCGGCTTCATAGCGTTG |
| 85A-F11 | GGGTGCCACGCCGAACACCGGTCCGGCGCCGCAGGGCGCCTAG |
| 85A-R11 | CTAGGCGCCCTGCGGCGCCGGACCGGTGTTCGGCGTGGCACCC |

Construction of pYA3941

| | |
|---|---|
| 85Am(93)-F1 | GCACGGCGACCGCGGAATTCTTTCTCGTCCGGGCCTG |
| 85Am(20)-R1 | GGTTCCAAAGATTTCTCGGTCGACGGCGCCCTGCGGCGCCG |

Construction of pYA4890

| | |
|---|---|
| SopE(N)-F1 | AAGGATCACCATGGGGACAAAAATAACTTTATCTCCCCAG |
| E2C(XN)-R1 | TTCTCTCACCCGGGAAAACAGCGCGGCCGCGCCTATCAGAAG |
| a85a(Nt)-F1 | GAATTGTGAGCGGCCGCCAATTTCACACAGGAAACAG |
| ECA(X)-R1 | CTGAAAATCTTCTCTCACCCGGGAAAACAGCCAAGC |

OmpC signal sequence

| | |
|---|---|
| PSOC-F1 | CATGGGGAAAGTTAAAGTACTGTCCCTCCTGGTACCAGCTCTGCTGGTGGC |
| | GGGCGCAGCGAATGCGGCTGAATTCCTGCAGCCAAGCTTCCCGGGT |
| PSOC-R1 | AGCTACCCGGGAAGCTTGGCTGCAGGAATTCAGCCGCATTCGCTGCGCCC |
| | GCCACCAGCAGAGCTGGTACCAGGAGGGACAGTACTTTAACTTTCCC |

pYA3814 construction

| | |
|---|---|
| 85A-F2m | GACCGCGAGATCTTTTTCTCGTCCGGGCTTG |

pYA3816 construction

| | |
|---|---|
| Mtb39A-F2 | GATCGATGGATATCACCTATGGTGGATTTCGGC |
| Mtb39A-R2 | CCAAGCTTCGATATCCTAGCCGGCC |

pYA4683 construction

| | |
|---|---|
| SopE2-F2 | GCATACCATGGTAAAAGGATGGTGACTAACATAACACTATCC |
| Mtb39A-R3 | CCAAGCTTCCTGCAGCTAGCCGGCC |

pYA4856 construction

| | |
|---|---|
| Mtb39A-F3 | CCACGAGAAATAGGGCCATGGAATGGTGGATTTCGG |

### Regulated Lysis Vectors.

Construction of pYA4890, pYA4891 and pYA4893 was described by Juarez-Rodriguez et al. (61). Plasmid pYA4683 was constructed by digesting pYA4589 (a derivative of pYA3681 in which the p15A on replaced the pBR on of pYA3681) with NcoI and PstI. The sopE2_{Nt}-ppe18 cassette was PCR-amplified with primers SopE2-F2 and Mtb39A-R3, digested with NcoI and PstI and ligated to pYA4589 to generate pYA4683. The sopE2_{Nt}-ppe18 cassette was also ligated to NcoI⁺ PstI-digested pYA3681 to generate pYA4851. The ppe gene alone was PCR-amplified with Mtb39A-F3 and Mtb39A-R3, digested with NcoI and PstI and ligated to pYA3681 digested with the same enzymes to form pYA4856. Plasmid pYA3816 was constructed by amplifying the ppe-18 gene with primers Mtb39A-F2 and Mtb39A-R2 and digesting the PCR product with EcoRV. The digested fragment was ligated to pYA3650 that had been digested and blunt-ended and this ligation resulted in the construction of pYA3816.

### Animal Experiment 1.

Groups of mice were immunized orally with RASVs χ11021(pYA4890), χ11021(pYA4891), χ11021(pYA4893), χ11021(pYA3681) and BSG on days 0, 7 and 49. Another group of mice was immunized by subcutaneous injection of 5×10⁴ cfu of Mycobacterium bovis BCG one time, at day 0. Blood samples were obtained by submandibular vein puncture 2 days before the first immunization and at day 77 after the first vaccination. Antibody titers were determined from these samples by ELISA. Four weeks after the last immunization (day 77), all mice were infected with an estimated inhaled dose of 100 cfu of M. tuberculosis H37Rv per lung, delivered by aerosol in an inhalation exposure system (Glas-Col, LLC, Terre Haute, Ind.). The mice were euthanized six weeks after challenge and the lungs and spleens were aseptically collected. Bacterial loads were determined by plating serial dilutions of whole-organ homogenates. Protection was defined as a bacterial load that was statistically significantly lower than the bacterial load in the BSG-dosed control group. ELISPOT assays were performed on spleen cells obtained from 3 mice from each group three weeks after the last immunization to measure production of interferon-γ-, TNF-α-, Interleukin-2 (IL-2)- and IL-4-secreting cells.

### Animal Experiment 2.

Groups of mice will be immunized orally with RASVs χ11021(pYA4956), χ11246 (pYA4683), χ11246 (pYA4851), χ11246 (pYA4856), χ11324(pYA4856), χ11327(pYA4856), χ11412(pYA3816) and a mixture of χ11246 (pYA4891)+χ11412(pYA3816) on days 0, 7 and 49. Another group of mice will be orally immunized on the same days with BSG. Another group of mice will be immunized once subcutaneously with 5×10⁴ cfu of M. bovis BCG on day 0. Blood samples will be obtained by submandibular vein puncture two days before the first immunization and at days 21 and 77 after the first immunization. Antibody titers will be determined by ELISA.

Four weeks after the last immunization (day 77), mice in all groups will be challenged with an aerosol dose of M. tuberculosis H37Rv, as described above. Lungs and spleens will be removed from 6 mice from each group 7 days after the last immunization, 21 days after challenge with M. tuberculosis and at 6 weeks after challenge. At six weeks after challenge, all mice in all groups will be euthanized; lungs and spleens will be aseptically removed and the tissues will be homogenized. Part of the homogenates of each organ will be serially diluted and plated to determine bacterial loads in the lungs and spleen. The remainder of the lung homogenates will be assayed for production of IFN-γ, TNF-α, IL-10, IL-17, effector CD4⁺ T cells, memory CD4⁺ T cells, effector CD8⁺ T cells and memory CD8⁺ T cells by flow cytometry.

### ELISA

ELISA assays were conducted as described previously (61). Total IgG, IgG1 and IgG2b antibody titers against Ag85A, ESAT-6, CFP-10 and Salmonella outer membrane proteins (SOMPs) were determined. Nunc Immunoplate Maxisorb F96 plates (Nalge Nunc. Rochester, N.Y., USA) were coated with purified Ag85A at 0.5 μg/well, ESAT-6 or CFP-10 at 1 μg/well or SOMPs at 0.5 μg/well suspended in 0.05 M carbonate-bicarbonate buffer, pH 9.6. Sera obtained from the same experimental group were pooled and serially diluted by two-fold dilutions from an initial dilution of 1:200 in PBS. ELISA assays were performed in triplicate, as described previously (42). Endpoint titers were expressed as the last sample dilution with an absorbance of 0.1 OD units above negative controls after 1 h of incubation.

### ELISPOT

The ELISPOT assay was performed using the ELISPOT kits (Mouse IFN-γ, TNF-α, IL-2, IL-4 ELISPOT Sets, eBioscience) according to the manufacturer's instructions, to enumerate the interferon-gamma (IFN-γ), tumor necrosis factor-alpha (TNF-α), interleukin-2 (IL-2) and IL-4 cytokine-secreting cells in the spleens of immunized and naïve mice. ELISPOT assays were conducted three weeks after the last immunization with the pool of spleens from three mice from the same group; these assays were also done in triplicate. Spleen cells from all groups of mice were incubated with the recombinant antigen at 1 μg/well or culture media at 37° C. in a humidified (5% CO₂-in-air) incubator. Splenocytes from mice immunized with the Salmonella strains χ9879 (pYA3620) and χ9879 (pYA3941) were incubated for 24 h (for INF-γ and TNF-α-secreting cells) or 48 h (for IL-2 and IL-14-secreting cells), while the spleen cells from mice immunized with the Salmonella χ11021 strains harboring independently each of the Asd⁺/MurA⁺ Lysis vector derivatives were incubated for 40 h (for INF-γ and TNF-α-secreting cells) or 66 h (for IL-2 and IL-14-secreting cells). The spots were counted using an automated ELISPOT plate reader (CTL Analyzers; Cellular Technology Ltd, Cleveland, Ohio).

### Flow Cytometry.

Preparation of Cells: Mice will be euthanized and the thoracic cavity opened. The lungs will be cleared of blood by perfusion through the pulmonary artery with 10 ml of ice-cold phosphate buffered saline (PBS) (Mediatech Inc, Manassas Va.) containing 50 U/ml of heparin (Sigma, St. Louis, Mo.). Lungs will be aseptically removed and placed in medium. The dissected lung tissue will be incubated with incomplete DMEM containing collagenase XI (0.7 mg/ml; Sigma-Aldrich) and type IV bovine pancreatic DNase (30 μg/ml; Sigma-Aldrich) for 30 minutes at 37° C. The digested lungs will be further disrupted by gently pushing the tissue through a cell strainer (BD Biosciences, Sparks, Md.). Red Blood Cells will be lysed with Gey's Solution, washed, and resuspended in complete DMEM. Total cell numbers will be determined by flow cytometry using Invitrogen CountBright absolute liquid counting beads, as described by the manufacturer (Invitrogen, Eugene, Oreg.).

Flow Cytometry for surface markers and intracellular cytokines: For flow cytometry analysis, single cell suspensions of lungs from each mouse will be resuspended in 1×PBS (Mediatech Inc, Manassas, Va.) containing 0.1% sodium azide. Cells will be incubated in the dark for 30 minutes at either 4° C. or 37° C. with predetermined optimal titrations of specific antibodies. Cell surface expression will be analyzed for CD4, CD44, CD62L, CD8, and CCR7 and the cytokines analyzed will be IFN-γ, TNF-α, IL-10, and IL-17. All antibodies and reagents will be purchased from BD Pharmingen (San Jose, Calif.), eBioscience (San Diego, Calif.), or Biolegend (San Diego, Calif.). All samples will be analyzed on a Becton Dickinson LSR II instrument, and data analyzed using FACSDiva v.6.1.1 software. Cells will be gated on lymphocytes based on characteristic forward- and side-scatter profiles. Individual cell populations will be identified according to the presence of specific fluorescence-labeled antibodies. All the analyses will be performed with an acquisition of a minimum of 100,000 events.

### Statistical Analysis.

Statistical analysis was performed using GraphPad Prism Software (GraphPad Software, San Diego, Calif.). Differences in antibody responses, cytokine secretion levels measured by ELISPOT, and bacterial load in the lungs and spleen between groups were determined by one-way analysis of variance ANOVA, followed by Tukey's Multiple Comparison Test. Differences with P values <0.05 were considered significant at the 95% confidence interval.

### Example 7

### Results of Animal Experiment 1

M. tuberculosis antigens ESAT-6, CFP-10 and Ag85A were produced in immunized mice by the regulated delayed lysis strain χ11021 harboring the lysis vectors pYA4890, pYA4891 and pYA4893. RASV χ11021 produces the SifA protein, thereby precluding escape of the RASV from the endosomal compartment and decreasing the likelihood that the M. tuberculosis antigens efficiently reach the cytoplasm of the antigen-presenting cells to enable efficient processing by the proteosome to elicit CMI responses. Mice immunized with χ11021(pYA4890), χ11021(pYA4891) or χ11021(pYA4893) produced higher levels of IgG2b antibodies against Ag85A than IgG1 antibodies. Spleens from these immunized animals had more IFN-γ- and TNF-α-producing cells than the non-immunized control mice, although mice immunized with χ11021(pYA4890) or χ11021(pYA4891) had stronger responses than mice immunized with χ11021(pYA4893). Importantly, mice immunized with each of these RASV constructs provided protection against aerosol challenge with virulent M. tuberculosis at a level equivalent to the protection afforded by the M. bovis BCG vaccine, which is considered the “gold standard” for vaccines against M. tuberculosis.

### Example 8

### Results from Animal Experiment 2

In animal experiment 2, the enhanced immune responses induced by delivery of M. tuberculosis antigens by Salmonella RASV strains that harbor mutations in sifA and the combination of ΔsifA, ΔtlpA, ΔsseL and ΔP_{hilA}::P_{trc ΔlacO888} hilA mutations may be assessed. The proposed experiments may also enable us to determine the types of immune responses elicited in mice immunized with RASV vaccines producing the antigen Mtb39A and the optimal means of delivery of this antigen by RASV systems. We may also determine whether or not delivery of four M. tuberculosis antigens as a mixture of two RASV strains (one delivering ESAT-6, CFP-10 and Ag85A as protein antigens delivered to the cytosol of antigen-presenting cells via a regulated delayed lysis strain and one delivering Mtb39A as a DNA vaccine via a regulated delayed lysis strain). Employing flow cytometry analyses of spleen cells from mice immunized with the RASV-M. tuberculosis vaccines may enable us to determine the subsets of T cells elicited, which may provide us with a more accurate assessment of the immune responses generated. Challenge of the immunized mice with aerosolized M. tuberculosis may enable us to determine the level of protection afforded by the RASV-M. tuberculosis vaccines, compared to the protection provided by the M. bovis BCG vaccine. It is anticipated, however, that delivery of M. tuberculosis antigens to the cytosol using Salmonella vaccine vectors with regulated delayed lysis occurring in the cytosol due to presence of a sifA deletion mutation may induce a superior T cell immune response that may effectively prevent infections by drug-sensitive and drug-resistant M. tuberculosis strains.

### References for Examples 7 and 8

55. Cooper, A M (2009) Cell-mediated immune responses in tuberculosis. Annu. Rev. Immunol. 27:393-422.
56. Cooper A M, Dalton D K, Stewart T A, Griffin J P, Russell D G, Orme I M (1993) Disseminated tuberculosis in interferon-γ-disrupted mice. J. Exp. Med. 178:2243-2247.
57. Dillon D C, Alderson M R, Day C H, Lewinsohn D M, Coler R, Bement T, Campos-Neto S, Skeiky Y A, Orme I M, Roberts A, Steen S, Dalemans W, Badaro R, Reed S G. (1999) Molecular characterization and human T-cell responses to a member of a novel Mycobacterium tuberculosis mtb39 gene family. Infect. Immun. 67:2941-2950.
58. D'Souza S, Rosseels V, Romano M, Tanghe A, Denis O, Jurion F, Castiglione N, Vanonckelen A, Palfliet K, Huygen K (2003) Mapping of murine Th1 helper T-cell epitopes of mycolyl transferases Ag85A, Ag85B and Ag85C from Mycobacterium tuberculosis. Infect. Immun. 71:483-493.
59. Flynn J L, Chan J, Triebold K J, Dalton D K, Stewart T A, Bloom B R (1993) An essential role for interferon-γ in resistance to Mycobacterium tuberculosis infection. J. Exp. Med. 178:2249-2254.
60. Havlir D and Barnes P (1999) Tueberculosis patients with human immunodeficiency virus infection. N. Engl. J. Med. 340:367-373.
61. Juárez-Rodriguez, M D, Yang J, Keder R, Alamuri P, Curtiss R III, Clark-Curtiss J E (2012) Live attenuated Salmonella vaccines displaying regulated delayed lysis and delayed antigen synthesis to confer protection against Mycobacterium tuberculosis. Infect. Immun. 80:815-831.
62. Lozes E, Huygen K, Content J, Denis O, Montgomery D L, Yawman A M, Vandenbussche P, Van Vooren J P, Drowart A, Ulmer J B, Liu M A (1997) Immunogenicity and efficacy of a tuberculosis DNA vaccine encoding the components of the secreted antigen 85 complex. Vaccine 15:830-8335
63. Mogues T, Goodrich M E, Ryan L, LaCourse R, North R J (2001) The relative importance of T cell subsets in immunity and immunopathology of airborne Mycobacterium tuberculosis infection in mice. J. Exp. Med. 193:271-280.
64. Orme I M (1987) The kinetics of emergence and loss of mediator T lymphocytes acquired in response to infection with Mycobacterium tuberculosis. J. Immunol. 138:293-298.
65. Orme I M (2011) Development of new vaccines and drugs for TB: limitations and potential strategic errors. Future Microbiol. 6:161-177.
66. Ottenhof T, Kumararante D, Casanova J (1998) Novel human immunodeficiencies reveal the essential role of type-1 cytokines in immunity to intracellular bacteria. Immunol. Today 19:491-494.
67. Skjot R L, Oettinger T, Rosenkrands I, Ravn P, Brock I, Jacobsen S, Andersen P (2000) Comparative evaluation of low molecular mass proteins from Mycobacterium tuberculosis identifies members of the ESAT-6 family as immunodominant T cell antigens. Infect. Immun. 68:214-220.
68. Sorensen A L, Nagai S, Houen G, Andersen P, Andersen A B (1995) Purification and characterization of a low molecular mass T-cell antigen secreted by Mycobacterium tuberculosis. Infect. Immun. 63:1710-1717.

1. A recombinant bacterium, wherein the bacterium has:
a) regulated expression of at least one nucleic acid encoding an antigen against a pathogen, and
b) at least one mutation allowing endosomal escape, such that the antigen is delivered to the cytosol and induces host cellular immunity against the pathogen.

2. The recombinant bacterium of claim 1, wherein the pathogen is influenza virus.

3. The recombinant bacterium of claim 1, wherein the antigen is the NP of influenza virus.

4. The recombinant bacterium of claim 1, wherein the antigen is the NP fused to one or more conserved T-cell epitopes of influenza virus.

5. The recombinant bacterium of claim 1, wherein the antigen is the NP147-155 epitope of influenza virus.

6. The recombinant bacterium of claim 1, wherein the pathogen is Mycobacterium tuberculosis.

7. The recombinant bacterium of claim 1, wherein the antigen is an antigen of Mycobacterium tuberculosis selected from the group consisting of ESAT-6, CFP-10, Ag85A, Ag85B, Ag85C, Mtb39A, FAP, Tb15.3, RfpA and RfpB.

8. The recombinant bacterium of claim 1, wherein cellular immunity is induced by endosomal escape.

9. The recombinant bacterium of claim 1, wherein cellular immunity is CD8 mediated immunity.

10. The recombinant bacterium of claim 1, wherein the cellular immunity is against a conserved epitope of the pathogen.

11. The recombinant bacterium of claim 1, wherein the antigen is known to contain a T cell epitope.

12. A vaccine composition, the composition comprising a recombinant bacterium of claim 1.

13. A method of inducing a cellular immune response against a pathogen, the method comprising administering a vaccine composition comprising a recombinant bacterium of claim 1 to a host.

14. A method for eliciting a cellular immune response in a host, the method comprising administering to the host an effective amount of a vaccine composition comprising a recombinant bacterium of claim 1.
